(19) United States
(12) Patent Application Publication
(10) Pub. No.: US 2024/0092803 A1
(43) Pub. Date: Mar. 21, 2024

(54) BRIDGED COMPOUNDS AS KRAS G12D INHIBITOR AND DEGRADER AND THE USE THEREOF

(71) Applicant: BEIGENE SWITZERLAND GMBH, Basel (CH)

(72) Inventors: Qi JI, Beijing (CN); Chao YU, Beijing (CN); Ce WANG, Beijing (CN); Hanzi SUN, Beijing (CN)

(21) Appl. No.: 18/260,654

(22) PCT Filed: Jan. 7, 2022
(86) PCT No.: PCT/CN2022/070675

§ 371 Date: Jul. 7, 2023

(30) Foreign Application Priority Data
Jan. 8, 2021 (WO) PCT/CN2021/070897

(51) Int. Cl. C07D 519/00; C07D 471/04

(52) U.S. Cl. CPC C07D 519/00; C07D 471/04

(57) ABSTRACT

The disclosure herein provides bridged compounds as well as their compositions and methods of use. The compounds disclosed herein inhibit KRAS G12D activity and are useful in the treatment of various diseases including cancer.

### FIELD OF THE INVENTION

The disclosure herein provides bridged compounds as well as their compositions and methods of use. The compounds disclosed herein inhibit KRAS G12D activity and are useful in the treatment of various diseases including cancer.

### BACKGROUND OF THE INVENTION

Ras is a family of proteins which are associated with cell membrane through their C-terminal membrane targeting region and well known as the molecular switch in intracellular signaling network (Cox A D, Der C J. Ras history: The saga continues. Small GTPases. 2010; 1(1):2-27). Ras proteins bind with either GTP or GDP and switch between “on” and “off” states. When Ras proteins bind with GDP, it is in the off (or inactive) state. And when Ras is switched on by certain growth promoting stimuli like growth factors, Ras proteins will be induced to exchange its bound GDP for a GTP and turn into on (or active) state (Malumbres M, Barbacid M. RAS oncogenes: the first 30 years. Nat Rev Cancer. 2003; 3(6):459-465). By switching to active state, Ras protein can interact with different downstream proteins and activate related signaling pathways (Berndt N, Hamilton A D, Sebti S M. Targeting protein prenylation for cancer therapy. Nat Rev Cancer. 2011; 11(11):775-791). Ras superfamily contains different subfamilies including Ras, Ral, Rap, Rheb, Rad, Rit and Miro (Wennerberg K, Rossman K L, Der C J. The Ras superfamily at a glance. J Cell Sci. 2005; 118(Pt 5):843-846). HRas, NRas and KRas are the most well studied proteins in Ras family since these proteins are the most common oncogenes in human cancers (O'Bryan JP. Pharmacological targeting of RAS: Recent success with direct inhibitors. Pharmacol Res. 2019; 139:503-511).

KRas is one of the most frequently mutated genes in human cancers. Based on data from Catalogue of Somatic Mutations (COSMIC) database, KRas mutation can be found in about 20% of human cancers, including pancreatic cancer, colorectal cancer, lung cancer, skin cancer etc. (O'Bryan J P. Pharmacological targeting of RAS: Recent success with direct inhibitors. Pharmacol Res. 2019; 139:503-511). And the most common KRas mutations are found at position G12 and G13 by blocking the GTPase activating proteins (GAP) stimulated GTP hydrolysis activity of KRas (Wang W, Fang G, Rudolph J. Ras inhibition via direct Ras binding—is there a path forward?. Bioorg Med Chem Lett. 2012; 22(18):5766-5776). That results in the over activation of KRas protein and ultimately leads to uncontrolled cell proliferation and cancer.

Among different cancers, pancreatic cancer is considered as the most KRas-addicted cancer type. KRas mutation is found in 94.1% of pancreatic ductal adenocarcinoma (PDAC). G12D (41%) and G12V (34%) mutations of KRas are the two most predominant mutations in all the KRas mutated PDAC (Waters A M, Der C J. KRAS: The Critical Driver and Therapeutic Target for Pancreatic Cancer. Cold Spring Harb Perspect Med. 2018; 8(9):a031435). In vivo data generated by mouse models proves that the progression and maintenance of pancreatic cancer are highly rely on the constitutive activation of KRas downstream signaling(Siveke J T, Schmid R M. Chromosomal instability in mouse metastatic pancreatic cancer—it's Kras and Tp53 after all. Cancer Cell. 2005; 7(5):405-407). Which indicates that mutated KRas protein is a highly attractive drug target for pancreatic cancer and also other cancers with KRas mutation. Since WT KRas protein also plays a critical role in the function of normal tissue and WT KRas function is demonstrated to be essential for adult hematopoiesis (Malumbres M, Barbacid M. RAS oncogenes: the first 30 years. Nat Rev Cancer. 2003; 3(6):459-465). It is highly deserved that a potential drug molecule can selectively inhibit mutated KRas protein in cancer cells and spare its WT companion in normal cells. Because KRas protein is generally considered as a non-druggable target, there is no therapeutics which can selectively target KRas protein with G12D mutation in clinic.

There are two strategies a drug molecule can adopt to selectively eliminate the over activated KRas signaling which induced by KRas mutations. One way is to directly bind with the mutated KRas protein, either by stabilizing its GDP bound form (the inactive form) or by blocking the interaction between GTP bound form and its downstream target protein. Another strategy is to hijack the protein degradation mechanism in cell and leverage E3 ligases' (like VHL, CRBN or IAPB) substrate specificity through a bi-functional molecule called Proteolysis targeting chimera (PROTAC)(Winter G E, Buckley D L, Paulk J, Roberts J M, Souza A, Dhe-Paganon S, Bradner J E. DRUG DEVELOPMENT. Phthalimide conjugation as a strategy for in vivo target protein degradation. Science. 2015 Jun. 19; 348(6241):1376-81). which can bind with both mutated KRas protein and E3 ligase, create interactions between those two proteins and induced KRas degradation.

Thus, KRas G12D mutation is a highly attractive target for pancreatic cancer and other cancers with this mutation. As such, small-molecule therapeutic agents that are capable to selectively bind with KRas G12D and inhibit its function would be very useful. And KRas G12D targeting bi-functional PROTAC is also an attractive strategy to target cancers with this mutation.

### SUMMARY OF THE INVENTION

In the first aspect, disclosed herein are bridged compounds of Formula (I), and the methods of use. The bridged compounds disclosed herein inhibit KRAS G12D activity and are useful in the treatment of various diseases including cancer. The first embodiment comprises the following aspects:
Aspect 1: A bridged compound of Formula (I):

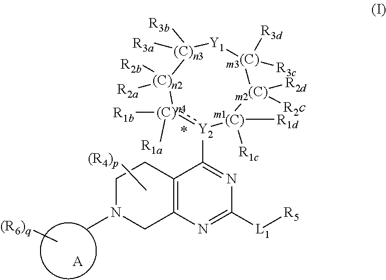

or a pharmaceutically acceptable salt thereof, or a stereoisomer thereof,
   wherein:
Ring A is an aryl group or a 5- to 7-membered monocyclic heteroaryl or 8- to 12-membered bicyclic heteroaryl group;
[symbol] is each independently a single bond or double bond;
Y₁ is —NH— or —C(R_{Y1a})(NHR_{Y1b})—;
Y₂ is N or CR_{Y2} in the case that [symbol] is a single bond; or Y₂ is C and R_{1b} is absent in the case that [symbol] is a double bond;
n1, n2, n3, m1, m2, and m3 are each independently 0 or 1, provided that at least one of n1, n2 and n3 is 1; and at least one of m1, m2 and m3 is 1;
p is 0, 1, 2, 3, 4, 5 or 6;
q is 0, 1, 2, 3, 4, 5, 6 or 7 provided that the valence theory is met;
R₁ₐ, R_{1b}, R₂ₐ, R_{2b}, R₃ₐ, R_{1c}, R_{1d}, R_{2c}, R₂ₐ, R_{3c}, R_{3d}, R_{Y1a}, R_{Y1b} and R_{Y2}, if present, are each independently hydrogen, halogen, —C₁₋₈alkyl, C₃₋₈cycloalkyl, 3- to 8-membered heterocyclyl, C6-C12aryl, 5- to 12-membered heteroaryl, —CN, —OR ₁ₑ, or —NR₁ₑR_{1f}; wherein each of —C₁₋₈alkyl, C₃₋₈cycloalkyl, 3- to 8-membered heterocyclyl, C₆-C₁₂aryl or 5- to 12-membered heteroaryl is optionally substituted with at least one substituent R_{1g};
at least one pair of (R₁ₐ and R_{1c}), (R₁ₐ and R_{2c}), (R₁ₐ and R_{3c}), (R₂ₐ and R_{1c}), (R₂ₐ and R_{2c}), (R₂ₐ and R_{3c}), (R₃ₐ and R_{1c}), (R₃ₐ and R_{2c}), (R₃ₐ and R_{3c}), (R_{Y1a} and R_{Y2}), (R_{Y1a} and R₁ₐ), (R_{Y1a} and R₂ₐ), (R_{Y1a} and R_{1c}), (R_{Y1a} and R_{2c}), (R_{Y1b} and R₁ₐ), (R_{Y1b} and R₂ₐ), (R_{Y1b} and R_{1c}), (R_{Y1b} and R_{2c}), and (R_{Y1b} and R_{Y2}) form a bridge containing one, two, three, or four —CH₂— moieties in addition to the two bridgeheads, wherein one of the —CH₂— moiety is optionally replaced with —O—, —S—or —NH— and wherein said bridge is optionally substituted with at least one substituent R_{1g};
optionally, (R_{Y1a} and R_{Y1b}), (R_{Y1a} and R₃ₐ), (R_{Y1a} and R_{3c}), (R_{Y1b} and R₃ₐ), or (R_{Y1b} and R_{3c}) form 3- to 12-membered ring, the said ring comprises 0-3 heteroatoms selected from nitrogen, sulfur and oxygen and the said bridge is optionally substituted with at least one substituent R_{1g};
R₁ₑ and R_{1f} are each independently selected from hydrogen, —C₁₋₈alkyl, —C₂₋₈alkenyl, —C₂₋₈alkynyl, C₁₋₈alkoxy-C₁₋₈alkyl-, C₃₋₈cycloalkyl, 3- to 8-membered heterocyclyl, C₆-C₁₂aryl or 5- to 12-membered heteroaryl;
R_{1g}, at each occurrence, is independently halogen, —C₁₋₈alkyl, —C₂₋₈alkenyl, —C₂₋₈alkynyl, —C₃₋₈cycloalkyl, 3- to 8-membered heterocyclyl, —C₆-C₁₂aryl, or 5- to 12-membered heteroaryl, —C₁₋₈haloalkyl, C₁₋₈alkoxy-C₁₋₈alkyl-, —CN, —OH, —NH₂, —C₁₋₈alkoxyl, —COOH, -or CO—C₁₋₈alkyl;
R₆ is hydrogen, halogen, —C₁₋₈alkyl, —C₂₋₈alkenyl, —C₂₋₈alkynyl, C₃₋₈cycloalkyl, 3- to 8-membered heterocyclyl, C₆-C₁₂aryl, 5- to 12-membered heteroaryl, —CN, oxo, —OR₆ₐ, —SR₆ₐ, —SO₂R₆ₐ, —SO₂R₆ₐ, —SO₂NR₆ₐR_{6b}, —COR₆ₐ, —CO₂R₆ₐ, —CONR₆ₐR_{6b}, —NR₆ₐR_{6b}, —NR₆ₐCOR_{6b}, —NR₆ₐCO₂R_{6b}, or —NR₆ₐ SO₂R_{6b}; each of —C₁₋₈alkyl, —C₂₋₈alkenyl, —C₂₋₈alkynyl, C₃₋₈cycloalkyl, 3- to 8-membered heterocyclyl, C₆-C₁₂aryl or 5- to 12-membered heteroaryl is optionally substituted with at least one substituent R_{6c};
R₆ₐ and R_{6b} are each independently selected from hydrogen, —C₁₋₈alkyl, —C₂₋₈alkenyl, —C₂₋₈alkynyl, C₁₋₈alkoxy-C₁₋₈alkyl-, C₃₋₈cycloalkyl, 3- to 8-membered heterocyclyl, C₆-C₁₂aryl or 5- to 12-membered heteroaryl,wherein each of —C₁₋₈alkyl, C₃₋₈cycloalkyl, 3- to 8-membered heterocyclyl, C₆-C₁₂aryl or 5- to 12-membered heteroaryl is optionally substituted with at least one substituent R_{6d};
R_{6c}, at each occurrence, is independently halogen, —C₁₋₈alkyl, —C₂₋₈alkenyl, —C₂₋₈alkynyl, —C₃₋₈cycloalkyl, 3- to 8-membered heterocyclyl, —C₆-C₁₂aryl, or 5- to 12-membered heteroaryl; or
two R₆ together with the atoms to which they are attached, form a 5, 6, 7, or 8-membered unsaturated (preferred aromatic) or saturated ring, said ring comprising 0-3 heteroatoms independently selected from nitrogen, oxygen or sulfur; said ring is optionally substituted with at least one substituent R_{6d};
R_{6d} is hydrogen, halogen, —C₁₋₈alkyl, —C₂₋₈alkenyl, —C₂₋₈alkynyl, C₃₋₈cycloalkyl, 3- to 8-membered heterocyclyl, C₆-C₁₂aryl, 5- to 12-membered heteroaryl, —CN, oxo, —OR₆ₑ, —SO₂R₆ₑ, —SO₂R₆ₑ, —SO₂NR₆ₑR_{6f}, —COR₆ₑ, —CO₂R₆ₑ, —CONR₆ₑR_{6f}, —NR₆ₑR_{6f}, —NR₆ₑCOR_{6f}, —NR₆ₑCO₂R_{6f}, or —NR₆ₑSO₂R_{6f};
each of C₁₋₈alkyl, —C₂₋₈alkenyl, —C₂₋₈alkynyl, C₃₋₈cycloalkyl, 3- to 8-membered heterocyclyl, C₆-C₁₂aryl or 5- to 12-membered heteroaryl is optionally substituted with at least one substituent R_{6g},
R₆ₑ and R_{6f} are each independently selected from hydrogen, —C₁₋₈alkyl, —C₂₋₈alkenyl, —C₂₋₈alkynyl, C₁₋₈alkoxy-C₁₋₈alkyl-, C₃₋₈cycloalkyl, 3- to 8-membered heterocyclyl, C₆-C₁₂aryl or 5- to 12-membered heteroaryl;
R_{6g}, at each occurrence, is independently halogen, —C₁₋₈alkyl, —C₂₋₈alkenyl, —C₂₋₈alkynyl, —C₃₋₈cycloalkyl, 3- to 8-membered heterocyclyl, —C₆-C₁₂aryl, or 5- to 12-membered heteroaryl;
R₄ is hydrogen, halogen, —C₁₋₈alkyl, C₃₋₈cycloalkyl, —C₂₋₈alkenyl, —C₂₋₈alkynyl, 3- to 8-membered heterocyclyl, C₆-C₁₂aryl, 5- to 12-membered heteroaryl, —CN, oxo, —OR₄ₐ, —SR₄ₐ, —SO₂R₄ₐ, —SO₂NR₄ₐR_{4b}, —COR₄ₐ, —CO₂R₄ₐ, —CONR₄ₐR_{4b}, —NR₄ₐR_{4b}, —NR₄ₐCOR_{4b}, —NR₄ₐCO₂R_{4b}, or —NR₄ₐSO₂R_{4b}; each of —C₁₋₈alkyl, C₃₋₈cycloalkyl, —C₂₋₈alkenyl, —C₂₋₈alkynyl, 3- to 8-membered heterocyclyl, C₆-C₁₂aryl or 5- to 12-membered heteroaryl is optionally substituted with at least one substituent R_{4c}, or two R₄ join each other to form spiro cycle or bicycle
R₄ₐ and R_{4b} are each independently selected from hydrogen, —C₁₋₈alkyl, —C₂₋₈alkenyl, —C₂₋₈alkynyl, C₁₋₈alkoxy-C₁₋₈alkyl-, C₃₋₈cycloalkyl, 3- to 8-membered heterocyclyl, C₆-C₁₂aryl or 5- to 12-membered heteroaryl; each of —C₁₋₈alkyl, —C₂₋₈alkenyl, —C₂₋₈alkynyl, C₁₋₈alkoxy-C₁₋₈alkyl-, C₃₋₈cycloalkyl, 3- to 8-membered heterocyclyl, C₆-C₁₂aryl or 5- to 12-membered heteroaryl is optionally substituted with at least one substituent R_{4d};
R_{4c} and R_{4d}, at each occurrence, are each independently halogen, —C₁₋₈alkyl, —C₂₋₈alkenyl, —C₂₋₈alkynyl, —C₃₋₈cycloalkyl, 3- to 8-membered heterocyclyl, —C₆-C₁₂aryl, or 5- to 12-membered heteroaryl;
L₁ is selected from a single bond, —O—, —NR^{L1a}—, —C(O)—, —C₁₋₈alkylene-, *^{L1}-O—C₁₋₈alkylene-**^{L1}, —C₃₋₈cycloalkylene-, *^{L1}-O—C₃₋₈cycloalkylene-**^{L1}, ^{L1}-O—C₁₋₈alkylene-NR^{L1a}-**^{L1}, ^{L1}-O—C₁₋₈alkylene-CO-**^{L1}, *^{L1}-C₁₋₈alkylene-O-**^{L1}, *^{L1}-C(O)-C₁₋₈-C(O)-C₁₋₈alkylene-**^{L1}, *^{L1}-C₁₋₈alkylene-C(O)-**^{L1}, *^{L1}-NR^{L1a}-C₁₋₈alkylene-**^{L1}, *^{L1}C₁₋₈alkylene-NR^{L1a}-**^{L1},

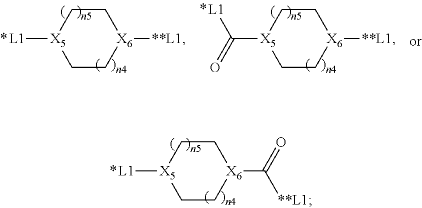

each of said —C₁₋₈alkylene-, *^{L1}-O—C₁₋₈alkylene-**^{L1}, —C₃₋₈cycloalkylene-, *^{L1}-O—C₃₋₈cycloalkylene-**^{L1}, *^{L1}-O—C₁₋₈alkylene-NR^{L1a}-**^{L1}, *^{L1}-O—C₁₋₈alkylene-CO-**^{L1}, *^{L1}-C₁₋₈alkylene-O-**^{L1}, *^{L1}-C(O)—C₁₋₈alkylene-**^{L1}, *^{L1}-C₁₋₈alkylene-C(O)-**^{L1}, *^{L1}-NR^{L1a}—C₁₋₈alkylene-**^{L1}, *^{L1}-C₁₋₈alkylene-NR^{L1a}-**^{L1},

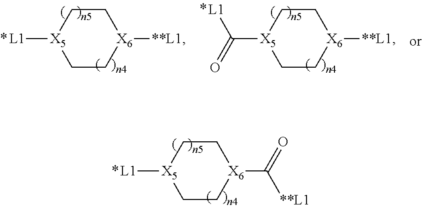

are optionally substituted with at least one R^{L1b};
wherein **^{L1} refers to the position attached to the

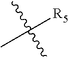

moiety, and *^{L1} refers to the position attached to the other side;
R^{L1a} is selected from hydrogen, —C₁₋₈alkyl —C₂₋₈alkenyl, —C₂₋₈alkynyl, C₃₋₈cycloalkyl, 3- to 8-membered heterocyclyl, C₆-C₁₂aryl or 5- to 12-membered heteroaryl, each of said —C₁₋₈alkyl, —C₂₋₈-alkenyl, —C₂₋₈alkynyl, C₃₋₈cycloalkyl, 3- to 8-membered heterocyclyl, C₆-C₁₂aryl or 5- to 12-membered heteroaryl is optionally substituted with at least one substituent R^{L1c};
each of said R^{L1b} and R^{L1c} are independently halogen, hydroxy, —C₁₋₈alkyl, —C₁₋₈alkoxy, —C₂₋₈alkenyl, —C₂₋₈alkynyl, C₃₋₈cycloalkyl, 3- to 8-membered heterocyclyl, C₆-C₁₂aryl or 5- to 12-membered heteroaryl; or
two R^{L1b} or two R^{L1c} together with the atoms to which they are attached, form a 3- to 6-membered unsaturated or saturated ring, said ring comprising 0-3 heteroatoms independently selected from nitrogen, oxygen or sulfur; said ring is optionally substituted with at least one substituent halogen, hydroxy, —C₁₋₈alkyl, —C₁₋₈alkoxy, —C₂₋₈alkenyl, —C₂₋₈alkynyl, C₃₋₈cycloalkyl, 3- to 8-membered heterocyclyl, C₆-C₁₂aryl or 5- to 12-membered heteroaryl;
each of X₅ and X₆ are selected from CH or N;
n4 and n5 are each independently 0, 1 or 2;
R₅ is hydrogen, halogen, —C₁₋₈alkyl, C₃₋₈cycloalkyl, 3- to 12-membered heterocyclyl, C₆-C₁₂aryl, 5- to 12-membered heteroaryl, oxo, —CN, —O R₅ₐ, —COR₅ₐ, —CO₂R₅ₐ, —CONRₐ₅R_{5b}, —NR₅ₐR_{5b}, —NR₅ₐCOR_{5b} or —NR₅ₐCO₂R_{5b}; wherein each of —C₁₋₈alkyl, C₃₋₈cycloalkyl, 3- to 12-membered heterocyclyl, C₆-C₁₂aryl or 5- to 12-membered heteroaryl is optionally substituted with at least one substituent R_{5c};
R₅ₐ and R_{5b} are each independently selected from hydrogen, —C₁₋₈alkyl, —C₂₋₈alkenyl, —C₂₋₈alkynyl, C₁₋₈alkoxy-C₁₋₈alkyl-, C₃₋₈cycloalkyl, 3- to 8-membered heterocyclyl, C₆-C₁₂aryl, 5- to 12-membered heteroaryl or oxo, wherein each of said —C₁₋₈alkyl, —C₂₋₈alkenyl, —C₂₋₈alkynyl, C₁₋₈alkoxy-C₁₋₈alkyl-, C₃₋₈cycloalkyl, 3- to 8-membered heterocyclyl, C₆-C₁₂aryl, 5- to 12-membered heteroaryl is optionally substituted with at least one substituent R_{5d} ; or
R₅ₐ and R_{5b} together with the carbon atoms to which they are attached, form a 3- to 8-membered unsaturated or saturated ring, said ring comprising 0-3 heteroatoms independently selected from nitrogen, oxygen or sulfur; said ring is optionally substituted with at least one substituent R_{5c};
R_{5c}, at each occurrence, is independently halogen, —C₁₋₈alkyl, —C₂₋₈alkenyl, —C₂₋₈alkynyl, —C₃₋₈cycloalkyl, 3- to 8-membered heterocyclyl, —C₆-C₁₂aryl, 5- to 12-membered heteroaryl, oxo, —CN, —OR₅ₑ, —COR₅ₑ, —CO₂R₅ₑ, —CONR₅ₑR_{5f}, —NR₅ₑR_{5f}, —NR₅ₑCOR_{5f} or —NR₅ₑCO₂R_{5f}, wherein each of said —C₁₋₈alkyl, —C₂₋₈alkenyl, —C₂₋₈alkynyl, —C₃₋₈cycloalkyl, 3- to 8-membered heterocyclyl, —C₆-C₁₂aryl, or 5- to 12-membered heteroaryl is optionally substituted with at least one substituent R_{5d}; or
two R₅ together with the carbon atoms to which they are attached, form a 3- to 8-membered unsaturated or saturated ring, said ring comprising 0-3 heteroatoms independently selected from nitrogen, oxygen or sulfur; said ring is optionally substituted with at least one substituent R_{5d}; or
R_{5d} is hydrogen, halogen, —C₁₋₈alkyl, C₃-C₈cycloalkyl, 3- to 8-membered heterocyclyl, C₆-C₁₂aryl, 5- to 12-membered heteroaryl, oxo, —CN, —OR_{5g}, —COR_{5g}, —CO₂R_{5g}, —CONR_{5g}R₅ₕ, —NR_{5g}R₅ₕ, —NR_{5g}COR₅ₕ or —NR_{5g}CO₂R₅ₕ; wherein each of —C¹⁻⁸alkyl, C₃-C₈cycloalkyl, 3- to 8-membered heterocyclyl, C₆-C₁₂aryl or 5- to 12-membered heteroaryl is optionally substituted with at least one substituent R₅ᵢ; R₅ₑ, R_{5f}, R_{5g}, R₅ₕ and R₅ᵢ are each independently selected from hydrogen, —C₁₋₈alkyl, —C₂₋₈alkenyl, —C₂₋₈alkynyl, C₁₋₈alkoxy-C₁₋₈alkyl-, C₃-C₈cycloalkyl, 3- to 8-membered heterocyclyl, C₆-C₁₂aryl or 5- to 12-membered heteroaryl.
Aspect 2: The compound of Aspect 1, wherein Y₁ is selected from —NH— or —C(R_{Y1a})(NH₂)—.
Aspect 3: The compound of any one of Aspects 1-2, wherein R₁ₐ, R_{1b}, R₂ₐ, R_{2b}, R₃ₐ, R_{3b}, R_{1c}, R_{1d}, R_{2c}, R_{2d}, R_{3c}, R_{3d}, R_{Y1a}, R_{Y1b} and R_{Y2}, if present, are each independently hydrogen, —F, —Cl, —Br, —I, methyl, ethyl, propyl, butyl, pentyl, hexyl, heptyl, octyl, cyclopropyl, cyclobutyl, cyclopentyl, cyclohexyl, cycloheptyl, cyclooctyl, 3- to 8-membered heterocyclyl, phenyl, 5- to 12-membered heteroaryl, —CN, —OR₁ₑ, —NR₁ₑR_{1f}; wherein each of methyl, ethyl, propyl, butyl, pentyl, hexyl, heptyl, octyl, cyclopropyl, cyclobutyl, cyclopentyl, cyclohexyl, cycloheptyl, cyclooctyl, 3- to 8-membered heterocyclyl, phenyl or 5- to 12-membered heteroaryl is optionally substituted with at least one substituent R_{1g},
   one pair of (R₁ₐ and R_{1c}, (R₁ₐ and R_{2c}), (R₁ₐ and R_{3c}), (R₂ₐ and R_{1c}), (R₂ₐ and R_{2c}), (R₂ₐ and R_{3c}), (R₃ₐ and R_{1c}), (R₃ₐ and R_{2c}), (R₃ₐ and R_{3c}), (R_{Y1a} and R_{Y2}), and (R_{Y1b} and R_{Y2}) form a bridge containing one, two, three, or four —CH₂— moieties in addition to the two bridgeheads, wherein one of the —CH₂— moiety is optionally replaced with —O—, —S—or —NH— and wherein said bridge is optionally substituted with at least one substituent R_{1g};
   R₁ₑ and R_{1f} are each independently selected from hydrogen, methyl, ethyl, propyl, butyl, pentyl, hexyl, heptyl, octyl, cyclopropyl, cyclobutyl, cyclopentyl, cyclohexyl, cycloheptyl, cyclooctyl, —C₂₋₈alkenyl, —C₂₋₈alkynyl, C₁₋₈alkoxy-C₁₋₈alkyl-, 3- to 8-membered heterocyclyl, phenyl or 5- to 12-membered heteroaryl;
   R_{1g}, at each occurrence, is independently —F, —Cl, —Br, —I, methyl, ethyl, propyl, butyl, pentyl, hexyl, heptyl, octyl, cyclopropyl, cyclobutyl, cyclopentyl, cyclohexyl, cycloheptyl, cyclooctyl, —C₂₋₈alkenyl, —C₂₋₈alkynyl, 3- to 8-membered heterocyclyl, phenyl, or 5- to 12-membered heteroaryl.
Aspect 4: The compound of any one of Aspects 1-3, wherein, the ring

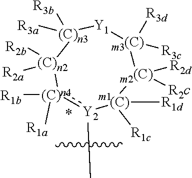

is a bridged bicyclic ring.
Aspect 5: The compound of any one of Aspects 1-4, wherein, the ring

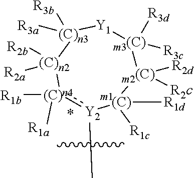

is selected from:
a) Y i is NH, Y₂ is N, [symbol] is a single bond, R₃ₐ and R_{3c} form a bridge containing one, two, or three —CH₂— moieties in addition to the two bridgeheads (nitrogen), wherein one of the —CH₂— moiety is optionally replaced with —O—, —S—or —NH— and wherein said bridge is optionally substituted with halogen (preferably F) or alkyl, and n3=m3=n1=m1=1 and n2=m2=0; or
b) Y₁ is NH, Y₂ is N, [symbol] is a single bond, R₂ₐ and R_{2c} form a bridge containing one, two, or three —CH₂— moieties in addition to the two bridgeheads (nitrogen), wherein one of the —CH₂— moiety is optionally replaced with —O—, —S—or —NH—, and n1=m1=n2=m2=n3=m3=1; or
c) Y₁ is NH, Y₂ is N, [symbol] is a single bond, R₁ₐ and R_{1c} form a bridge containing one, two, or three —CH₂— moieties in addition to the two bridgeheads (nitrogen), wherein one of the —CH₂— moiety is optionally replaced with —O—, —S—or —NH—, and n3=m3=n1=m1=1 and n2=m2=0; or
d) Y₁ is NH, Y₂ is N, [symbol] is a single bond, R₁ₐ and R_{1c} form a bridge containing one, two, or three —CH₂— moieties in addition to the two bridgeheads (nitrogen), wherein one of the —CH₂— moiety is optionally replaced with —O—, —S—or —NH—, and n2=m2=n3=m3=0 and n1=m1=1; or
e) Y₁ is NH, Y₂ is CH, [symbol] is a single bond, or Y₂ is C, [symbol] is a double bond, R₂ₐ and R_{1c} form a bridge containing one, two, or three —CH₂— moieties in addition to the two bridgeheads (one nitrogen and one carbon atom), wherein one of the —CH₂— moiety is optionally replaced with —O—, —S—or —NH—, and n1=n2=n3=m1=1, and m2=m3=0; or
f) Y₁ is NH, Y₂ is CH, [symbol] is a single bond, or Y₂ is C, [symbol] is a double bond, R₃ₐ and R_{3c} form a bridge containing one, two, or three —CH₂— moieties in addition to the two bridgeheads (one nitrogen and one carbon atom), wherein one of the —CH₂— moiety is optionally replaced with —O—, —S—or —NH—, and n1=m1=n3=m3=1, and n2=m2=0; or n3=m3=m1=1, and n1=n2=m2=0; or
g) Y₁ is CH, Y₂ is CH, [symbol] is a single bond, R_{Y1a} and R_{Y2} form a bridge containing one, two, or three —CH₂— moieties in addition to the two bridgeheads (two carbon atoms), wherein one of the —CH₂— moiety is optionally replaced with —O—, —S—or —NH—, and n1=m1=n3=m3=0, and n2=m2=1; or
h) Y₁ is NH, Y₂ is N, [symbol] is a single bond, R₃ₐ and R_{1c} form a bridge containing one, two, or three —CH₂— moieties in addition to the two bridgeheads (nitrogen), wherein one of the —CH₂— moiety is optionally replaced with —O—, —S—or —NH—, n1=m1=n3=m3=1, and n2=m2=0; or
i) Y₁ is NH, Y₂ is —CH(NH₂), [symbol] is a single bond, R₁ₐ and R_{1c} form a bridge containing one, two, or three —CH₂— moieties in addition to the two bridgeheads (one nitrogen and one carbon atom), wherein one of the —CH₂— moiety is optionally replaced with —O—, —S—or —NH—, n1=m1=n3=1, and n2=m2=m3=0.
Aspect 6: The compound of any one of Aspects 1-5, wherein the

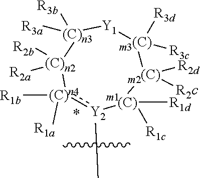

moiety is selected from

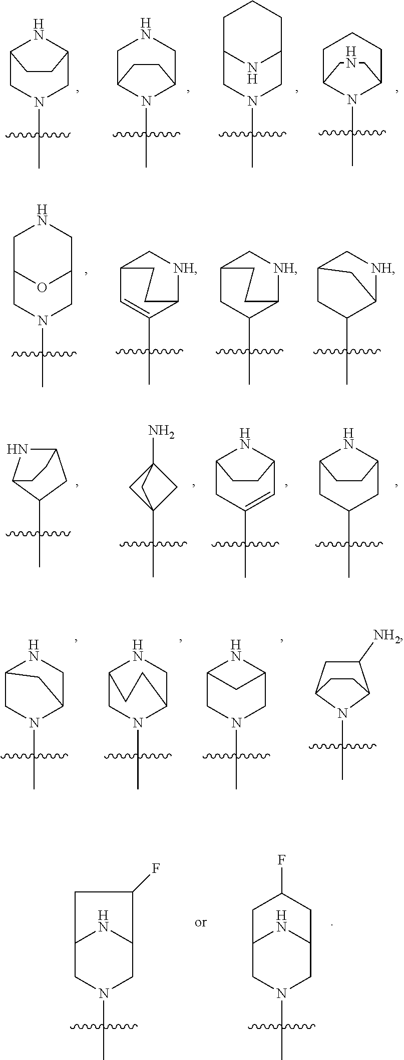

Aspect 7: The compound of any one of Aspects 1-6, wherein R₆ is hydrogen, —F, —Cl, —Br, —I, methyl, ethyl, propyl, butyl, pentyl, hexyl, heptyl, octyl, vinyl, propylenyl, allyl, butenyl, butadienyl, pentenyl, pentadienyl, hexenyl, hexadienyl, ethynyl, propynyl, butynyl, pentynyl, hexynyl, cyclopropyl, cyclobutyl, cyclopentyl, cyclohexyl, cycloheptyl, cyclooctyl, oxazolidinyl, imidazolidinyl, thiazolidinyl, pyrazolidinyl, morpholinyl, piperidinyl, piperazinyl, oxazinyl, imidazolyl, thiazolyl, oxazolyl, pyridyl, pyrimidinyl, pyrazinyl, pyridazinyl, phenyl, pyrrolyl, imidazolyl, pyrazolyl, thiazolyl, oxazolyl, triazolyl, thiophenyl, furanyl, pyridyl, pyrimidinyl, pyrazinyl, —CN, oxo, —OR₆ₐ, —SR₆ₐ or —NR₆ₐR_{6b}; each of methyl, ethyl, propyl, butyl, pentyl, hexyl, heptyl, octyl, cyclopropyl, cyclobutyl, cyclopentyl, cyclohexyl, cycloheptyl, cyclooctyl, oxazolidinyl, imidazolidinyl, thiazolidinyl, pyrazolidinyl, morpholinyl, piperidinyl, piperazinyl, oxazinyl, imidazolyl, thiazolyl, oxazolyl, pyridyl, pyrimidinyl, pyrazinyl, pyridazinyl, phenyl, pyrrolyl, imidazolyl, pyrazolyl, thiazolyl, oxazolyl, triazolyl, thiophenyl, furanyl, pyridyl, pyrimidinyl or pyrazinyl is optionally substituted with at least one substituent R_{6c},
   R₆ₐ and R_{6b} are each independently selected from hydrogen, methyl, ethyl, propyl, butyl, pentyl, hexyl, heptyl, octyl, —C₂₋₈alkenyl, —C₂₋₈alkynyl, C₁₋₈alkoxy-C₁₋₈alkyl-, cyclopropyl, cyclobutyl, cyclopentyl, cyclohexyl, cycloheptyl, cyclooctyl, 3- to 8-membered heterocyclyl, phenyl or 5- to 12-membered heteroaryl; wherein each of methyl, ethyl, propyl, butyl, pentyl, hexyl, heptyl, octyl, —C₂₋₈alkenyl, —C₂₋₈alkynyl, C₁₋₈alkoxy-C₁₋₈alkyl-, cyclopropyl, cyclobutyl, cyclopentyl, cyclohexyl, cycloheptyl, cyclooctyl, 3- to 8-membered heterocyclyl, phenyl or 5- to 12-membered heteroaryl is optionally substituted with at least one substituent R_{6d};
   R_{6c}, at each occurrence, is independently —F, —Cl, —Br, —I, methyl, ethyl, propyl, butyl, pentyl, hexyl, heptyl, octyl, —C₂₋₈alkenyl, —C₂₋₈alkynyl, cyclopropyl, cyclobutyl, cyclopentyl, cyclohexyl, cycloheptyl, cyclooctyl, 3- to 8-membered heterocyclyl, phenyl, or 5- to 12-membered heteroaryl; or two R₆ together with the atoms to which they are attached, form a 5-or 6-membered unsaturated (preferred aromatic) or saturated ring, said ring comprising 0, 1, 2, or 3 heteroatoms independently selected from nitrogen, oxygen or sulfur; said ring is optionally substituted with at least one substituent R_{6d};
   R_{6d} is hydrogen, —F, —Cl, —Br, —I, methyl, ethyl, propyl, butyl, pentyl, hexyl, heptyl, octyl, cyclopropyl, cyclobutyl, cyclopentyl, cyclohexyl, cycloheptyl, cyclooctyl, vinyl, propylenyl, allyl, butenyl, butadienyl, pentenyl, pentadienyl, hexenyl, hexadienyl, ethynyl, propynyl, butynyl, pentynyl, hexynyl, 3- to 8-membered heterocyclyl, phenyl, 5- to 12-membered heteroaryl, —CN, oxo, —OR₆ₑ or —NR₆ₑR_{6f}; each of methyl, ethyl, propyl, butyl, pentyl, hexyl, heptyl, octyl, cyclopropyl, cyclobutyl, cyclopentyl, cyclohexyl, cycloheptyl, cyclooctyl, vinyl, propylenyl, allyl, butenyl, butadienyl, pentenyl, pentadienyl, hexenyl, hexadienyl, ethynyl, propynyl, butynyl, pentynyl, hexynyl, 3- to 8-membered heterocyclyl, phenyl or 5- to 12-membered heteroaryl is optionally substituted with at least one substituent R_{6g},
   R₆ₑ and R_{6f} are each independently selected from hydrogen, methyl, ethyl, propyl, butyl, pentyl, hexyl, heptyl, octyl, —C₂₋₈alkenyl, —C₂₋₈alkynyl, C₁₋₈alkoxy-C₁₋₈alkyl-, cyclopropyl, cyclobutyl, cyclopentyl, cyclohexyl, cycloheptyl, cyclooctyl, 3- to 8-membered heterocyclyl, phenyl or 5- to 12-membered heteroaryl;
   R₆g , at each occurrence, is independently —F, —Cl, —Br, —I, methyl, ethyl, propyl, butyl, pentyl, hexyl, heptyl, octyl, cyclopropyl, cyclobutyl, cyclopentyl, cyclohexyl, cycloheptyl, cyclooctyl, —C₂₋₈alkenyl, —C₂₋₈alkynyl, 3- to 8-membered heterocyclyl, phenyl, or 5- to 12-membered heteroaryl
   preferably R₆ is hydrogen, —F, —Cl, —Br, —I, phenyl, methyl, —CF₃, —OCHF₂, —OCF₃, ethyl, vinyl, ethynyl, propyl, butyl, pentyl, —OH, —OMe, —OEt, —SH or —NH₂.

Preferably, R₆ is —OH, —CN, —NH₂, —F, —Cl, —Br, —I, —CH₃, —CH₂CH₃, —CH₂CH₂CH₃, —CF₃, —CHF₂, —CH₂ F, —CF₂ CH₃, —CF₂CF₃, —OCHF₂, —OCF₃, methoxy, ethoxy, propoxy, isopropoxy, butoxy, tert-butoxy, pentoxy, hexoxy, oxazolidinyl, imidazolidinyl, thiazolidinyl, pyrazolidinyl, morpholinyl, piperidinyl, piperazinyl, oxazinyl, imidazolyl, thiazolyl, oxazolyl, pyridyl, pyrimidinyl, pyrazinyl, pyridazinyl, phenyl, pyrrolyl, imidazolyl, pyrazolyl, thiazolyl, oxazolyl, triazolyl, thiophenyl, furanyl, pyridyl, pyrimidinyl, pyrazinyl, ethynyl, propynyl, butynyl, pentynyl, hexynyl, cyclopropyl, cyclobutyl, cyclopentyl, cyclohexyl, cycloheptyl, cyclooctyl, methyl, ethyl, propyl, butyl, pentyl, hexyl, heptyl, octyl, vinyl, propylenyl, allyl, butenyl, butadienyl, pentenyl, pentadienyl, hexenyl, hexadienyl.
Aspect 8: The compound of any one of Aspects 1-7, wherein Ring A is an aryl group selected from phenyl or naphthyl substituted with one or two R₆. In some embodiments, R₆ is selected from —CN, OH, —CF₃, —CHF₂, —CH₂F, —CF₂CH₃, —CF₂CF₃, aryl, ethynyl, propynyl, butynyl, pentynyl, hexynyl, cyclopropyl, cyclobutyl, cyclopentyl, cyclohexyl, cycloheptyl, cyclooctyl, methyl, ethyl, propyl, butyl, pentyl, hexyl, heptyl, octyl, vinyl, propylenyl, allyl, butenyl, butadienyl, pentenyl, pentadienyl, hexenyl, hexadienyl, methoxy, ethoxy, propoxy, isopropoxy, butoxy, tert-butoxy, pentoxy, or hexoxy.
Aspect 9: The compound of any one of Aspects 1-8, wherein Ring A is a 5- to 7-membered monocyclic heteroaryl or 8- to 12-membered bicyclic heteroaryl group substituted with one or two R₆. In some embodiments, R₆ is selected from —CN, OH, NH₂, —CF₃, —CHF₂, —CH₂F, —CF₂CH₃, —CF₂CF₃, aryl, ethynyl, propynyl, butynyl, pentynyl, hexynyl, cyclopropyl, cyclobutyl, cyclopentyl, cyclohexyl, cycloheptyl, cyclooctyl, methyl, ethyl, propyl, butyl, pentyl, hexyl, heptyl, octyl, vinyl, propylenyl, allyl, butenyl, butadienyl, pentenyl, pentadienyl, hexenyl, hexadienyl, methoxy, ethoxy, propoxy, isopropoxy, butoxy, tert-butoxy, pentoxy, or hexoxy. In some preferred embodiments, ring A is pyridyl or isoquinolinyl.
Aspect 10: The compound of any one of Aspects 1-9, wherein the

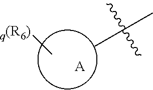

moiety is

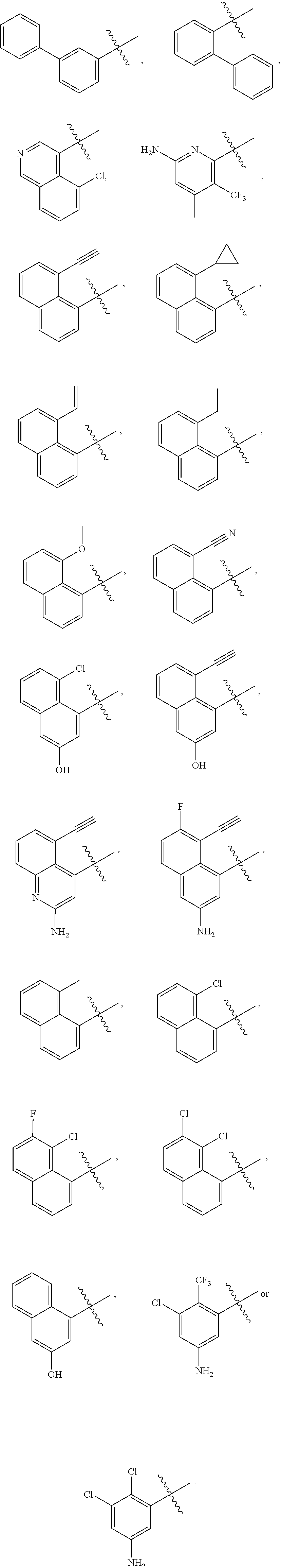

Aspect 11: The compound of any one of Aspects 1-10, wherein R₄ is hydrogen, —F, —Cl, —Br, —I, methyl, ethyl, propyl, butyl, pentyl, hexyl, heptyl, cyclopropyl, cyclobutyl, cyclopentyl, cyclohexyl, cycloheptyl, cyclooctyl, vinyl, propylenyl, allyl, butenyl, butadienyl, pentenyl, pentadienyl, hexenyl, hexadienyl, ethynyl, propynyl, butynyl, pentynyl, hexynyl, 3- to 8-membered heterocyclyl, phenyl, 5- to 12-membered heteroaryl, —CN, oxo, —OR₄ₐ, —SR₄ₐ, —SO₂R₄ₐ, —SO₂NR₄ₐR_{4b}, —COR₄ₐ, —CO₂R₄ₐ, —CONR₄ₐR_{4b}, —NR₄ₐR_{4b}, —NR₄ₐCOR_{4b}, —NR₄ₐCO₂R_{4b}, or —NR₄ₐSO₂R_{4b}; each of methyl, ethyl, propyl, butyl, pentyl, hexyl, heptyl, cyclopropyl, cyclobutyl, cyclopentyl, cyclohexyl, cycloheptyl, cyclooctyl, vinyl, propylenyl, allyl, butenyl, butadienyl, pentenyl, pentadienyl, hexenyl, hexadienyl, ethynyl, propynyl, butynyl, pentynyl, hexynyl, 3- to 8-membered heterocyclyl, phenyl or 5- to 12-membered heteroaryl is optionally substituted with at least one substituent R_{4c},
   R₄ₐ and R_{4b} are each independently selected from hydrogen, methyl, ethyl, propyl, butyl, pentyl, hexyl, heptyl, cyclopropyl, cyclobutyl, cyclopentyl, cyclohexyl, cycloheptyl, cyclooctyl, vinyl, propylenyl, allyl, butenyl, butadienyl, pentenyl, pentadienyl, hexenyl, hexadienyl, ethynyl, propynyl, butynyl, pentynyl, hexynyl, C₁₋₈alkoxy-C₁₋₈alkyl-, 3- to 8-membered heterocyclyl, phenyl or 5- to 12-membered heteroaryl; each of methyl, ethyl, propyl, butyl, pentyl, hexyl, heptyl, cyclopropyl, cyclobutyl, cyclopentyl, cyclohexyl, cycloheptyl, cyclooctyl, vinyl, propylenyl, allyl, butenyl, butadienyl, pentenyl, pentadienyl, hexenyl, hexadienyl, ethynyl, propynyl, butynyl, pentynyl, hexynyl, C₁₋₈alkoxy-C₁₋₈alkyl-, 3- to 8-membered heterocyclyl, phenyl or 5- to 12-membered heteroaryl is optionally substituted with at least one substituent R_{4d};

R_{4c} and R_{4d}, at each occurrence, are each independently —F, —Cl, —Br, —I, methyl, ethyl, propyl, butyl, pentyl, hexyl, heptyl, vinyl, propylenyl, allyl, butenyl, butadienyl, pentenyl, pentadienyl, hexenyl, hexadienyl, ethynyl, propynyl, butynyl, pentynyl, hexynyl, cyclopropyl, cyclobutyl, cyclopentyl, cyclohexyl, cycloheptyl, cyclooctyl, 3- to 8-membered heterocyclyl, —C₆-C₁₂aryl, or 5- to 12-membered heteroaryl.
Aspect 12: The compound of any one of Aspects 1-11, wherein R₄ is hydrogen, —F, —Cl, —Br —I, methyl, ethyl, propyl, isopropyl, isobutyl, tert-butyl, —CF₃, —CHF₂, —CH₂F, —OCHF₂, —OCF₃, cyclopropyl, cyclobutyl, cyclopentyl, vinyl, propylenyl or allyl.
Aspect 13: The compound of any one of Aspects 1-12, wherein L₁ is selected from a single bond, —O—, —NR^{L1a}—, —C(O)—, —CH₂—, —CH₂CH₂—, —CH₂CH₂CH₂—, —CH₂CH₂ CH₂ CH₂—, —CH₂CH₂CH₂CH₂CH₂—, —CH₂CH₂CH₂CH₂CH₂CH₂—, —CH₂CH₂CH₂CH₂CH₂CH₂CH₂—, —CH₂CH₂CH₂CH₂CH₂CH₂CH₂CH₂—, *^{L1}-O—CH₂-**^{L1}, *^{L1}-O—CH₂CH₂-**^{L1}, *^{L1}-O—CH₂CH₂CH₂- **^{L1}, *^{L1}-O—CH₂CH₂CH₂CH₂-**^{L1}, *^{L1}-O—CH₂CH₂CH₂CH₂CH₂-**^{L1}, *^{L1}-O—CH₂CH₂CH₂CH₂CH₂CH₂- **^{L1}, *^{L1}-O—CH₂CH₂CH₂CH₂CH₂CH₂CH₂-**^{L1}, *^{L1}-O—CH₂CH₂CH₂CH₂CH₂CH₂CH₂CH₂-**^{L1}, —C₃₋₈cycloalkylene-, *^{L1}-O—C₃-C₈cycloalkylene-**^{L1}, *^{L1}-O—CH₂—NR^{L1a}-**^{L1}, *^{L1}-O—CH₂CH₂—NR^{L1}-**^{L1}, *^{L1}-O—CH₂CH₂CH₂—NR^{L1a}-**^{L1}*^{L1}-O—CH₂CH₂CH₂CH₂—NR^{L1a}**^{L1}, *^{L1}-O—CH₂CH₂CH₂CH₂CH₂—NR^{L1a}-**^{L1}, *^{L1}-O—CH₂CH₂CH₂CH₂CH₂CH₂—NR^{L1a}-**^{L1}, *^{L1}-O—CH₂CH₂CH₂CH₂CH₂CH₂CH₂—NR^{L1a}-**^{L1}, *^{L1}-O—CH₂CH₂CH₂CH₂CH₂CH₂CH₂CH₂—NR^{L1a}-**^{L1}, *^{L1}-CH₂—CO-**^{L1}, *^{L1}-O—CH₂CH₂—CO-**^{L1}, *^{L1}-O—CH₂CH₂CH₂—CO -**^{L1}, *^{L1}-O—CH₂CH₂CH₂CH₂—CO-**^{L1}, *^{L1}-O—CH₂CH₂CH₂CH₂CH₂—CO-**^{L1}, *^{L1}-O—CH₂CH₂CH₂CH₂CH₂CH₂—CO-**^{L1}, *^{L1}-O—CH₂CH₂CH₂CH₂CH₂CH₂CH₂—CO -**^{L1}, *^{L1}-O—CH₂CH₂CH₂CH₂CH₂CH₂CH₂CH₂—CO-**^{L1}, *^{L1}-CH₂—O-**^{L1}, *^{L1}-CH₂CH₂—O-**^{L1}, *^{L1}-CH₂CH₂CH₂—O-**^{L1}, *^{L1}-CH₂CH₂CH₂CH₂—O-**^{L1}, *^{L1}-CH₂CH₂CH₂CH₂CH₂—O-**^{L1}, *^{L1}CH₂CH₂CH₂CH₂CH₂CH₂—O-**^{L1}, *^{L1}-CH₂CH₂CH₂CH₂CH₂CH₂CH₂—O-**^{L1}, *^{L1}-CH₂CH₂CH₂CH₂CH₂CH₂CH₂CH₂—O-**^{L1}, *^{L1}-C(O)—CH₂-**^{L1}, *^{L1}-C(O)—CH₂CH₂-**^{L1}, *^{L1}-C(O)—CH₂CH₂CH₂-**^{L1}, *^{L1}-C(O)—CH₂CH₂CH₂CH₂-**^{L1}, *^{L1}-C(O)—CH₂CH₂CH₂CH₂CH₂-**^{L1}, *^{L1}-C(O)—CH₂CH₂CH₂CH₂CH₂CH₂-**^{L1}, *^{L1}--C(O)—CH₂CH₂CH₂CH₂CH₂CH₂CH₂-**^{L1}, *^{L1}-C(O)—CH₂CH₂CH₂CH₂CH₂CH₂CH₂CH₂-**^{L1}, *^{L1}-CH₂—C(O)-**^{L1}, *^{L1}-CH₂CH₂—C(O)-**^{L1}, *^{L1}-CH₂CH₂—C(O)-**^{L1}, *^{L1}-CH₂CH₂CH₂—C(O)-**^{L1}, *^{L1}-CH₂CH₂CH₂CH₂—C(O)-**^{L1}, *^{L1}-CH₂CH₂CH₂CH₂CH₂—C(O)-**^{L1}, *^{L1}-CH₂CH₂CH₂CH₂CH₂CH₂—C(O)-**^{L1}, *^{L1}-CH₂CH₂CH₂CH₂CH₂CH₂CH₂—C(O)-**^{L1}, *^{L1}-CH₂CH₂CH₂CH₂CH₂CH₂CH₂CH₂—C(O)-**^{L1}, *^{L1}-CH₂CH₂—NR^{L1a}-**^{L1}, *^{L1}-CH₂CH₂CH₂—NR^{L1a}-**^{L1}, *^{L1}-CH₂CH₂CH₂CH₂—NR^{L1a}-**^{L1}, *^{L1}-CH₂CH₂CH₂CH₂CH₂—NR^{L1a}-**^{L1}, *^{L1}-CH₂CH₂CH₂CH₂CH₂CH₂—NR^{L1a}-**^{L1}, *^{L1}-CH₂CH₂CH₂CH₂CH₂CH₂CH₂—NR^{L1a}-**^{L1}, *^{L1}-CH₂CH₂CH₂CH₂CH₂CH₂CH₂CH₂—NR^{L1a}-**^{L1}, *^{L1}-NR^{L1a}—CH₂-**^{L1}, *^{L1}--NR^{L1a}—CH₂CH₂-**^{L1}, *^{L1}-NR^{L1a}—CH₂CH₂CH₂-**^{L1}, *^{L1}-NR^{L1a}—CH₂CH₂CH₂CH₂-**^{L1}, *^{L1}-NR^{L1a}—CH₂CH₂CH₂CH₂CH₂-**^{L1}, *^{L1}-NR^{L1a}—CH₂CH₂CH₂CH₂CH₂CH₂-**^{L1}, *^{L1}-NR^{L1a}—CH₂CH₂CH₂CH₂CH₂CH₂CH₂-**^{L1}, *^{L1}-NR^{L1a}—CH₂CH₂CH₂CH₂CH₂CH₂CH₂CH₂-**^{L1},

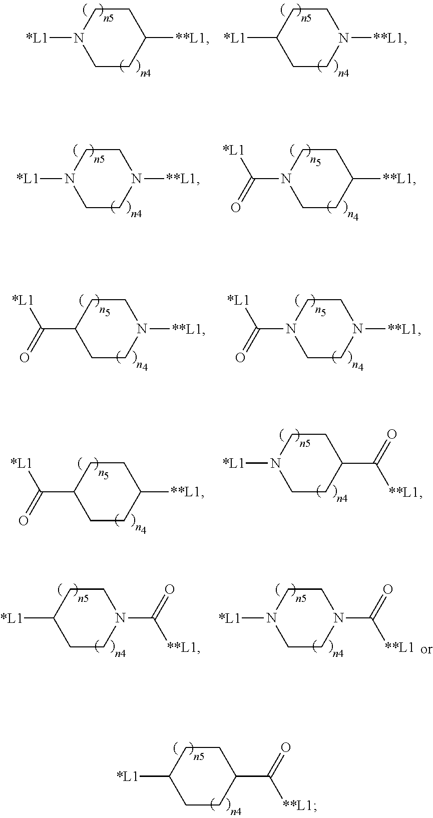

each of said —CH₂—, —CH₂CH₂—, —CH₂CH₂CH₂—, —CH₂CH₂CH₂CH₂—, —CH₂CH₂CH₂CH₂CH₂—, —CH₂CH₂CH₂CH₂CH₂CH₂, —CH₂CH₂CH₂CH₂CH₂CH₂CH₂—, —CH₂CH₂CH₂CH₂CH₂CH₂CH₂CH₂—, *^{L1}-O—CH₂-**^{L1}, *^{L1}-O—CH₂CH₂-**^{L1}, *^{L1}-O—CH₂CH₂CH₂-**^{L1}, *^{L1}-O—CH₂CH₂CH₂CH₂-**^{L1}, *^{L1}-O—CH₂CH₂CH₂CH₂CH₂-**^{L1}, *^{L1}-O—CH₂CH₂CH₂CH₂CH₂CH₂-**^{L1}, *^{L1}-O—CH₂CH₂CH₂CH₂CH₂CH₂CH₂-**^{L1}, *^{L1}-O—CH₂CH₂CH₂CH₂CH₂CH₂CH₂CH₂-**^{L1}, —C₃-C₈cycloalkylene-, -**^{L1}-O—C-C₈cycloalkylene-**^{L1}, *^{L1}-O—CH₂—NR-**^{L1a}-^{**L1}, *^{L1}-O—CH₂CH₂—NR-**^{L1a}-^{**L1}, *^{L1}-O—CH₂CH₂CH₂—NR-**^{L1a}-^{**L1}, *^{L1}-O—CH₂CH₂CH₂CH₂—NR-**^{L1a}-^{**L1}, *^{L1}-O—CH₂CH₂CH₂CH₂CH₂—NR-**^{L1a}-^{**L1}, *^{L1}-O—CH₂CH₂CH₂CH₂CH₂CH₂CH₂CH₂—NR-**^{L1a}-^{**L1}, *^{L1}-O—CH₂—CI-**^{L1}, *^{L1}-O—CH₂CH₂—CO—-**^{L1}, *^{L1}-O—CH₂CH₂CH₂—CO—-**^{L1}, *^{L1}-O—CH₂CH₂CH₂CH₂—CO—-**^{L1}, *^{L1}-O—CH₂CH₂CH₂CH₂CH₂—CO—-**^{L1}, *^{L1}-O—CH₂CH₂CH₂CH₂CH₂CH₂-**^{L1}, *^{L1}-O—CH₂CH₂CH₂CH₂CH₂CH₂CH₂—CO—-**^{L1}, *^{L1}-O—CH₂CH₂CH₂CH₂CH₂CH₂CH₂CH₂—CO—-**^{L1}, *^{L1}-CH₂—O-**^{L1}, *^{L1}-CH₂CH₂—O-**^{L1}, *^{L1}-CH₂CH₂CH₂—O-**^{L1}, *^{L1}-CH₂CH₂CH₂CH₂—O-**^{L1}, *^{L1}-CH₂CH₂CH₂CH₂CH₂—O-**^{L1}, *^{L1}-CH₂CH₂CH₂CH₂CH₂CH₂—O-**^{L1}, *^{L1}-CH₂CH₂CH₂CH₂CH₂CH₂CH₂—O-**^{L1}, *^{L1}-CH₂CH₂CH₂CH₂CH₂CH₂CH₂CH₂—O-**^{L1}, *^{L1}-C(O)—CH₂-**^{L1}, *^{L1}-C(O)—CH₂CH₂-**^{L1}, *^{L1}-C(O)—CH₂CH₂CH₂-**^{L1}, *^{L1}-C(O)—CH₂CH₂CH₂CH₂-**^{L1}, *^{L1}-C(O)—CH₂CH₂CH₂CH₂CH₂-**^{L1}, *^{L1}-C(O)—CH₂CH₂CH₂CH₂CH₂CH₂-**^{L1}, *^{L1}-C(O)—CH₂CH₂CH₂CH₂CH₂CH₂CH₂-**^{L1}, *^{L1}-C(O)—CH₂CH₂CH₂CH₂CH₂CH₂CH₂CH₂-**^{L1}, *^{L1}-CH₂—C(O)-**^{L1}, *^{L1}-CH₂CH₂—C(O)-**^{L1}, *^{L1}-CH₂CH₂CH₂—C(O)-**^{L1}, *^{L1}-CH₂CH₂CH₂CH₂—C(O)-**^{L1}, *^{L1}-CH₂CH₂CH₂CH₂CH₂—C(O)-**^{L1}, *^{L1}-CH₂CH₂CH₂CH₂CH₂CH₂—C(O)-**^{L1}, *^{L1}-CH₂CH₂CH₂CH₂CH₂CH₂CH₂—C(O)-**^{L1}, *^{L1}-CH₂CH₂CH₂CH₂CH₂CH₂CH₂CH₂—C(O)-**^{L1}, *^{L1}-CH₂—NR^{L1a}-^{**L1}, *^{L1}-CH₂CH₂—NR^{L1a}-^{**L1}, *^{L1}-CH₂CH₂CH₂—NR^{L1a}-^{**L1}, *^{L1}-CH₂CH₂CH₂CH₂—NR^{L1a}-^{**L1}, *^{L1}-CH₂CH₂CH₂CH₂CH₂—NR^{L1a}-^{**L1}, *^{L1}-CH₂CH₂CH₂CH₂CH₂CH₂—NR^{L1a}-^{**L1}, *^{L1}-CH₂CH₂CH₂CH₂CH₂CH₂CH₂—NR^{L1a}-^{**L1}, *^{L1}-CH₂CH₂CH₂CH₂CH₂CH₂CH₂CH₂—NR^{L1a}-^{**L1}, *^{L1}-NR^{L1a}—CH₂-**^{L1}, *^{L1}-NR^{L1a}—CH₂CH₂-**^{L1}, *^{L1}-NR^{L1a—CH}₂CH₂CH₂-**^{L1}, *^{L1}-CH₂CH₂CH₂CH₂-**^{L1}, *^{L1}-CH₂CH₂CH₂CH₂CH₂-**^{L1}, *^{L1}-NR^{L1a}—CH₂CH₂CH₂CH₂CH₂CH₂-**^{L1}, *^{L1}-NR^{L1a}-CH₂CH₂CH₂CH₂CH₂CH₂CH₂-**^{L1}, *^{L1}-NR^{L1a}-CH₂CH₂CH₂CH₂CH₂CH₂CH₂CH₂-**^{L1},

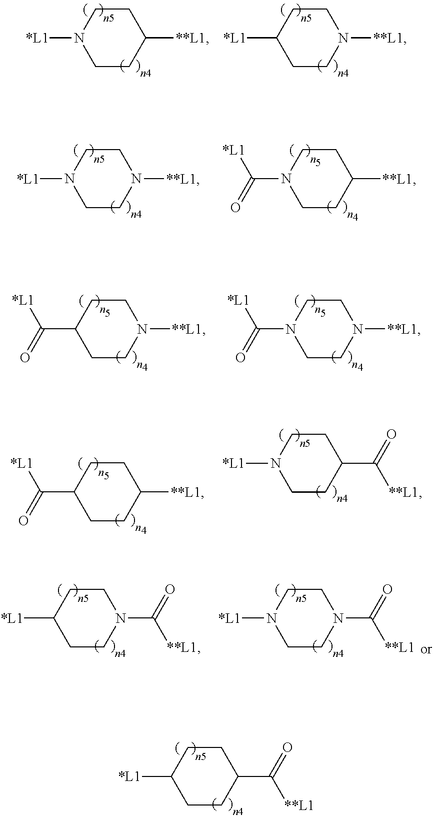

are optionally substituted with at least one R^{L1b}.
n4 and n5 are each independently 0 or 1;
R^{L1a} is selected from hydrogen, —C¹⁻⁸alkyl, —C₂₋₈alkenyl, —C₂₋₈alkynyl, C₃-C₈cycloalkyl, 3- to 8-membered heterocyclyl, C₆-C₁₂aryl or 5- to 12-membered heteroaryl, each of said —C¹⁻⁸alkyl, —C₂₋₈alkenyl, —C₂₋₈alkynyl, C₃-C₈cycloalkyl, 3- to 8-membered heterocyclyl, C₆-C₁₂aryl or 5- to 12-membered heteroaryl is optionally substituted with at least one substituent R^{L1c};
each of said R^{L1b} and R^{L1c} are independently halogen, hydroxy, —C₁₋₈alkyl, —C₁₋₈alkoxy, —C₂₋₈alkenyl, —C₂₋₈alkynyl, C₃-C₈cycloalkyl, 3- to 8-membered heterocyclyl, C₆-C₁₂aryl or 5- to 12-membered heteroaryl; or two R^{L1b} or two R^{L1c} together with the atoms to which they are attached, form a 3- to 6-membered unsaturated or saturated ring, said ring comprising 0-3 heteroatoms independently selected from nitrogen, oxygen or sulfur; said ring is optionally substituted with at least one substituent halogen, hydroxy, —C₁₋₈alkyl, —C₁₋₈alkoxy, —C₂₋₈alkenyl, —C₂₋₈alkynyl, C₃-C₈cycloalkyl, 3- to 8-membered heterocyclyl, C₆-C₁₂aryl or 5- to 12-membered heteroaryl.
Aspect 14: The compound of any one of Aspects 1-13, wherein L₁ is selected from

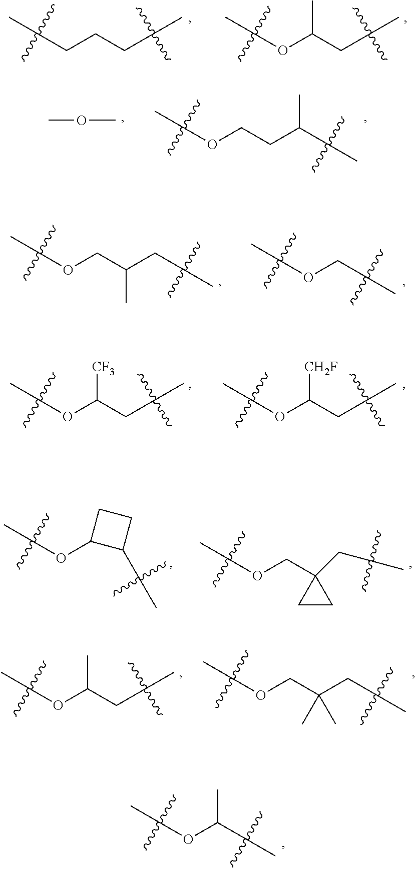

a single bond,

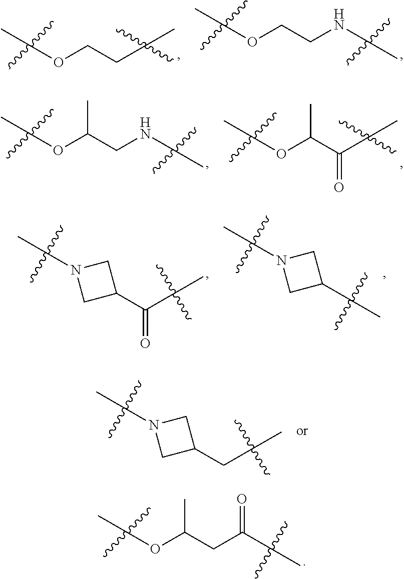

Aspect 15: The compound of any one of Aspects 1-14, wherein R₅ is hydrogen, —F, —Cl, —Br, —I, methyl, ethyl, propyl, butyl, pentyl, hexyl, heptyl, octyl, cyclopropyl, cyclobutyl, cyclopentyl, cyclohexyl, cycloheptyl, cyclooctyl, tetrahydrofuranyl, tetrahydropyranyl, 7- to 9-membered spiro-heterocylic ring comprising one or two or three nitrogen atoms as the ring members; oxazolidinyl, imidazolidinyl, thiazolidinyl, pyrazolidinyl, morpholinyl, piperidinyl, piperazinyl, oxazinyl, imidazolyl, thiazolyl, oxazolyl, pyridyl, pyrimidinyl, pyrazinyl, pyridazinyl, phenyl, pyrrolyl, imidazolyl, pyrazolyl, thiazolyl, oxazolyl, triazolyl, thiophenyl, furanyl, pyridyl, pyrimidinyl, pyrazinyl, phenyl, pyrrolidinyl, octahydroquinolizinyl, hexahydro-1H-pyrrolizinyl, oxo, —CN, —O R₅ₐ, —COR₅ₐ, —CO₂R₅ₐ, —CONR₅ₐR_{5b}, —NR₅ₐR_{5b}, —NR₅ₐCOR₅b or —NR₅ₐCO₂R_{5b}; wherein each of methyl, ethyl, propyl, butyl, pentyl, hexyl, heptyl, octyl, cyclopropyl, cyclobutyl, cyclopentyl, cyclohexyl, cycloheptyl, cyclooctyl, tetrahydrofuranyl, tetrahydropyranyl, 7- to 9-membered spiro-heterocylic ring comprising one or two or three nitrogen atoms as the ring members, oxazolidinyl, imidazolidinyl, thiazolidinyl, pyrazolidinyl, morpholinyl, piperidinyl, piperazinyl, oxazinyl, imidazolyl, thiazolyl, oxazolyl, pyridyl, pyrimidinyl, pyrazinyl, pyridazinyl, phenyl, pyrrolyl, imidazolyl, pyrazolyl, thiazolyl, oxazolyl, triazolyl, thiophenyl, furanyl, pyridyl, pyrimidinyl, pyrazinyl or phenyl is optionally substituted with at least one substituent R_{5c};
   R₅ₐ and R_{5b} are each independently selected from hydrogen, methyl, ethyl, propyl, butyl, pentyl, hexyl, heptyl, —C₂₋₈alkenyl, —C₂₋₈alkynyl, C₁₋₈alkoxy-C₁₋₈alkyl-, octyl, cyclopropyl, cyclobutyl, cyclopentyl, cyclohexyl, cycloheptyl, cyclooctyl, oxazolidinyl, imidazolidinyl, thiazolidinyl, pyrazolidinyl, morpholinyl, piperidinyl, piperazinyl, oxazinyl, imidazolyl, thiazolyl, oxazolyl, pyridyl, pyrimidinyl, pyrazinyl, pyridazinyl, phenyl, pyrrolyl, imidazolyl, pyrazolyl, thiazolyl, oxazolyl, triazolyl, thiophenyl, furanyl, pyridyl, pyrimidinyl, pyrazinyl or oxo; wherein each of methyl, ethyl, propyl, butyl, pentyl, hexyl, heptyl, —C₂₋₈alkenyl, —C₂₋₈alkynyl, C₁₋₈alkoxy-C₁₋₈alkyl-, octyl, cyclopropyl, cyclobutyl, cyclopentyl, cyclohexyl, cycloheptyl, cyclooctyl, oxazolidinyl, imidazolidinyl, thiazolidinyl, pyrazolidinyl, morpholinyl, piperidinyl, piperazinyl, oxazinyl, imidazolyl, thiazolyl, oxazolyl, pyridyl, pyrimidinyl, pyrazinyl, pyridazinyl, phenyl, pyrrolyl, imidazolyl, pyrazolyl, thiazolyl, oxazolyl, triazolyl, thiophenyl, furanyl, pyridyl, pyrimidinyl, pyrazinyl is optionally substituted with at least one substituent R_{5d}; or
   R₅ₐ and R_{5b} together with the carbon atoms to which they are attached, form a 3- to 8-membered unsaturated or saturated ring, said ring comprising 0, 1, 2, or 3 heteroatoms independently selected from nitrogen, oxygen or sulfur; said ring is optionally substituted with at least one substituent R_{5c};
   R_{5c}, at each occurrence, is independently halogen, —F, —Cl, —Br, —I, methyl, ethyl, propyl, butyl, pentyl, hexyl, heptyl, octyl, cyclopropyl, cyclobutyl, cyclopentyl, cyclohexyl, cycloheptyl, cyclooctyl, oxazolidinyl, imidazolidinyl, thiazolidinyl, pyrazolidinyl, morpholinyl, piperidinyl, piperazinyl, oxazinyl, imidazolyl, thiazolyl, oxazolyl, pyridyl, pyrimidinyl, pyrazinyl, pyridazinyl, phenyl, pyrrolyl, imidazolyl, pyrazolyl, thiazolyl, oxazolyl, triazolyl, thiophenyl, furanyl, pyridyl, pyrimidinyl, pyrazinyl, —C₂₋₈alkenyl, —C₂₋₈alkynyl, oxo, —CN, —OR₅ₑ, —COR₅ₑ, —CO₂R₅ₑ, —CONR₅ₑR_{5f}, —NR₅ₑR_{5f}, —NR₅ₑCOR_{5f} or —NR₅ₑCO₂R_{5f}; or
   two R₅ together with the carbon atoms to which they are attached, form a 3- to 8-membered unsaturated or saturated ring, said ring comprising 0, 1, 2, or 3 heteroatoms independently selected from nitrogen, oxygen or sulfur; said ring is optionally substituted with at least one substituent R_{5d}; R_{5d} is hydrogen, —F, —Cl, —Br, —I, methyl, ethyl, propyl, butyl, pentyl, hexyl, heptyl, octyl, cyclopropyl, cyclobutyl, cyclopentyl, cyclohexyl, cycloheptyl, cyclooctyl, oxazolidinyl, imidazolidinyl, thiazolidinyl, pyrazolidinyl, morpholinyl, piperidinyl, piperazinyl, oxazinyl, imidazolyl, thiazolyl, oxazolyl, pyridyl, pyrimidinyl, pyrazinyl, pyridazinyl, phenyl, pyrrolyl, imidazolyl, pyrazolyl, thiazolyl, oxazolyl, triazolyl, thiophenyl, furanyl, pyridyl, pyrimidinyl, pyrazinyl, oxo, —CN, —OR_{5g}, —COR_{5g}, —CO₂R_{5g}, —CONR_{5g}R₅ₕ, —NR_{5g}R₅ₕ, —NR_{5g}COR₅ₕ or —NR_{5g}CO₂R₅ₕ; wherein each of methyl, ethyl, propyl, butyl, pentyl, hexyl, heptyl, octyl, cyclopropyl, cyclobutyl, cyclopentyl, cyclohexyl, cycloheptyl, cyclooctyl, oxazolidinyl, imidazolidinyl, thiazolidinyl, pyrazolidinyl, morpholinyl, piperidinyl, piperazinyl, oxazinyl, imidazolyl, thiazolyl, oxazolyl, pyridyl, pyrimidinyl, pyrazinyl, pyridazinyl, phenyl, pyrrolyl, imidazolyl, pyrazolyl, thiazolyl, oxazolyl, triazolyl, thiophenyl, furanyl, pyridyl, pyrimidinyl or pyrazinyl is optionally substituted with at least one substituent R₅ᵢ;
   R₅ₑ, R_{5f}, R_{5g}, R₅ₕ and R₅ᵢ are each independently selected from hydrogen, methyl, ethyl, propyl, butyl, pentyl, hexyl, heptyl, octyl, cyclopropyl, cyclobutyl, cyclopentyl, cyclohexyl, cycloheptyl, cyclooctyl, oxazolidinyl, imidazolidinyl, thiazolidinyl, pyrazolidinyl, morpholinyl, piperidinyl, piperazinyl, oxazinyl, imidazolyl, thiazolyl, oxazolyl, pyridyl, pyrimidinyl, pyrazinyl, pyridazinyl, phenyl, pyrrolyl, imidazolyl, pyrazolyl, thiazolyl, oxazolyl, triazolyl, thiophenyl, furanyl, pyridyl, pyrimidinyl, pyrazinyl, —C₂₋₈alkenyl, —C₂₋₈alkynyl, C₁₋₈alkoxy-C₁₋₈alkyl- or C₃₋₈cycloalkyl.
Aspect 16: The compound of any one of Aspects 1-15, wherein R₅ is

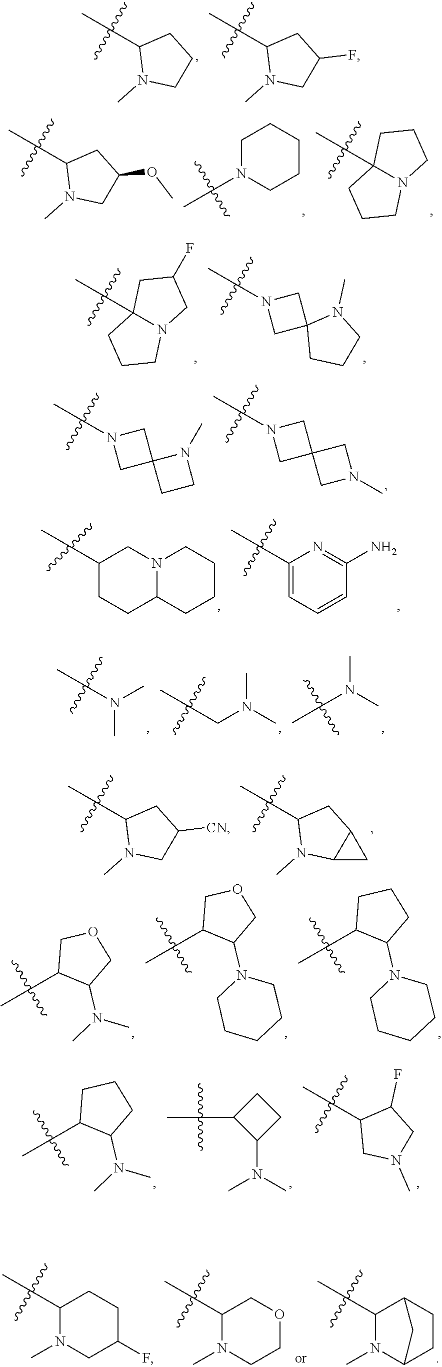

Aspect 17: The compound disclosed herein selected from

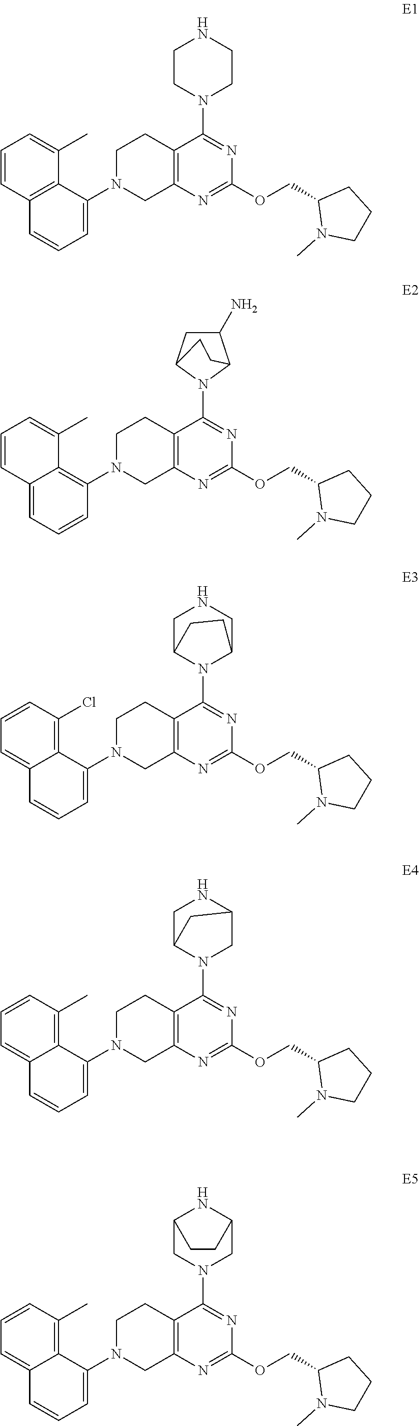 [SMILES: Cc1cccc2cccc(N3CCc4c(nc(OC[C@@H]5CCCN5C)nc4N4CCNCC4)C3)c12 ; Cc1cccc2cccc(N3CCc4c(nc(OC[C@@H]5CCCN5C)nc4N4C5CCC4C(N)C5)C3)c12 ; CN1CCC[C@H]1COc1nc2c(c(N3C4CCC3CNC4)n1)CCN(c1cccc3cccc(Cl)c13)C2 ; Cc1cccc2cccc(N3CCc4c(nc(OC[C@@H]5CCCN5C)nc4N4CC5CC4CN5)C3)c12 ; Cc1cccc2cccc(N3CCc4c(nc(OC[C@@H]5CCCN5C)nc4N4CC5CCC(C4)N5)C3)c12]

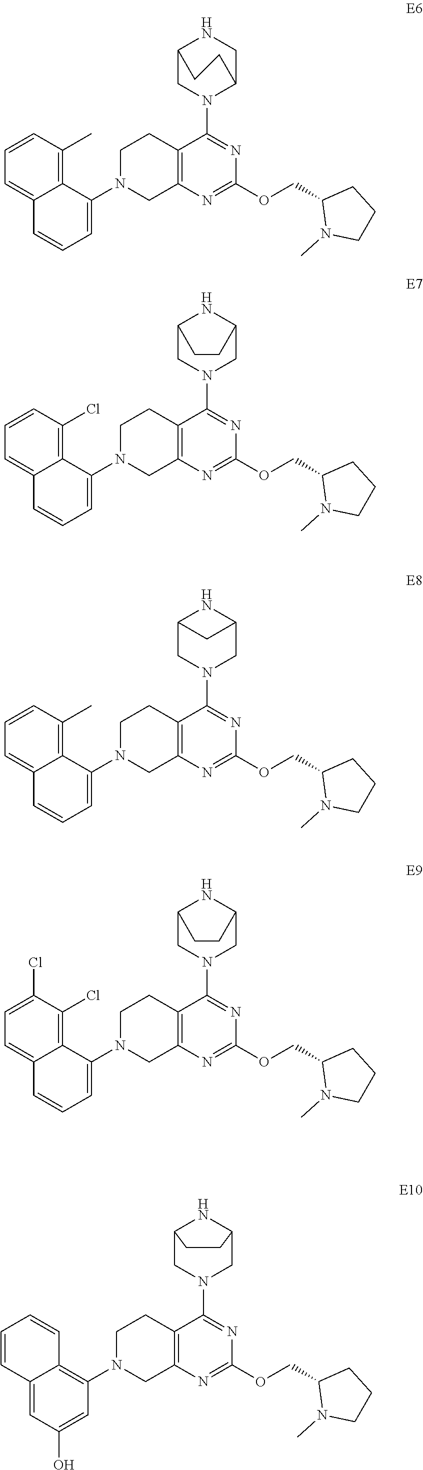 [SMILES: Cc1cccc2cccc(N3CCc4c(nc(OC[C@@H]5CCCN5C)nc4N4CC5CCC4CN5)C3)c12 ; CN1CCC[C@H]1COc1nc2c(c(N3CC4CCC(C3)N4)n1)CCN(c1cccc3cccc(Cl)c13)C2 ; Cc1cccc2cccc(N3CCc4c(nc(OC[C@@H]5CCCN5C)nc4N4CC5CC(C4)N5)C3)c12 ; CN1CCC[C@H]1COc1nc2c(c(N3CC4CCC(C3)N4)n1)CCN(c1cccc3ccc(Cl)c(Cl)c13)C2 ; CN1CCC[C@H]1COc1nc2c(c(N3CC4CCC(C3)N4)n1)CCN(c1cc(O)cc3ccccc13)C2]

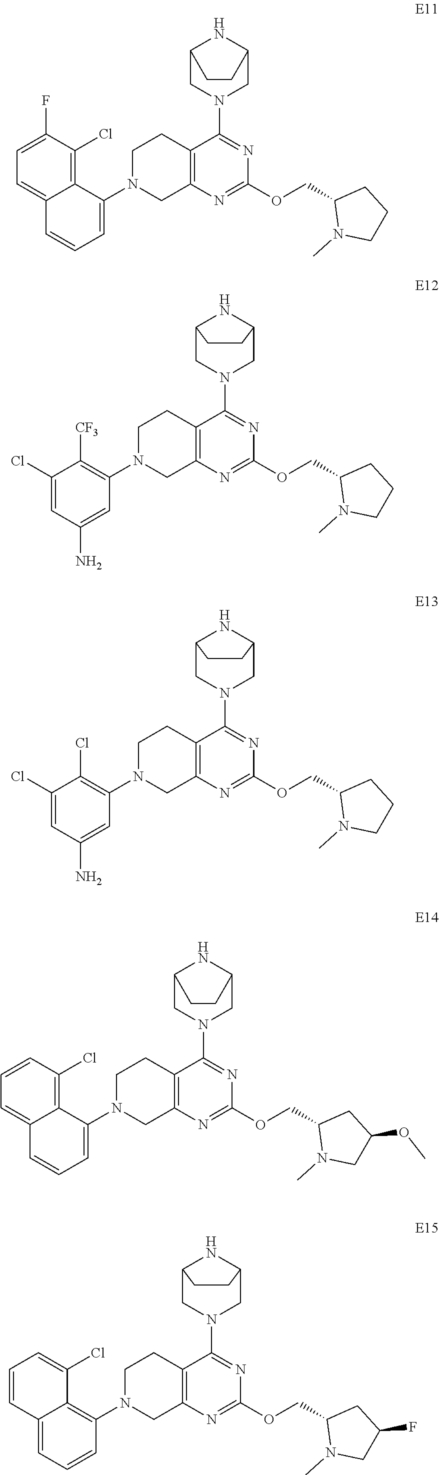 [SMILES: CN1CCC[C@H]1COc1nc2c(c(N3CC4CCC(C3)N4)n1)CCN(c1cccc3ccc(F)c(Cl)c13)C2 ; CN1CCC[C@H]1COc1nc2c(c(N3CC4CCC(C3)N4)n1)CCN(c1cc(N)cc(Cl)c1C(F)(F)F)C2 ; CN1CCC[C@H]1COc1nc2c(c(N3CC4CCC(C3)N4)n1)CCN(c1cc(N)cc(Cl)c1Cl)C2 ; CO[C@@H]1C[C@@H](COc2nc3c(c(N4CC5CCC(C4)N5)n2)CCN(c2cccc4cccc(Cl)c24)C3)N(C)C1 ; CN1C[C@H](F)C[C@H]1COc1nc2c(c(N3CC4CCC(C3)N4)n1)CCN(c1cccc3cccc(Cl)c13)C2]

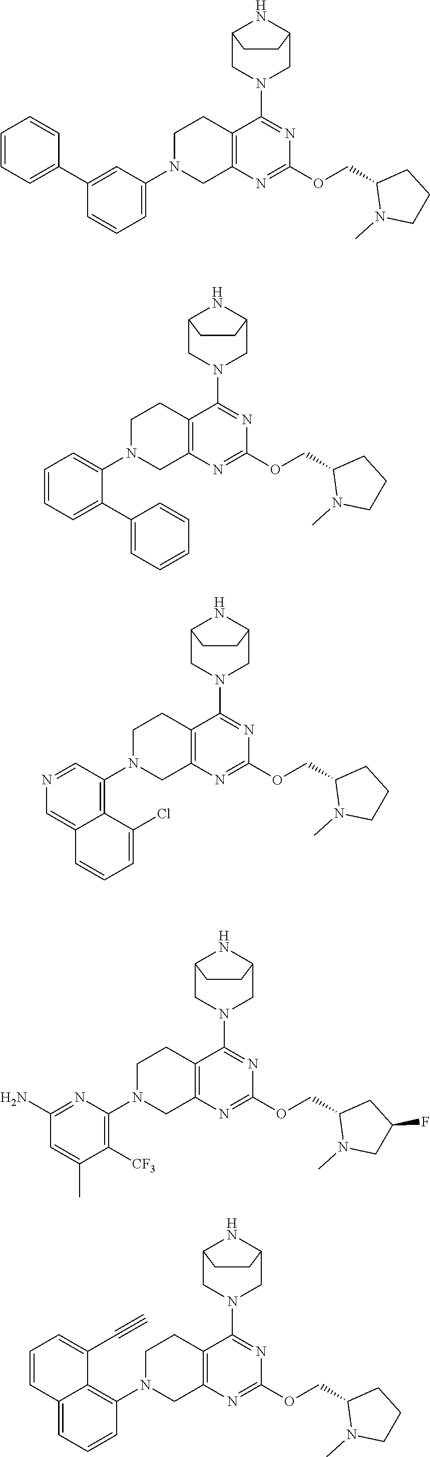 [SMILES: CN1CCC[C@H]1COc1nc2c(c(N3CC4CCC(C3)N4)n1)CCN(c1cccc(-c3ccccc3)c1)C2 ; CN1CCC[C@H]1COc1nc2c(c(N3CC4CCC(C3)N4)n1)CCN(c1ccccc1-c1ccccc1)C2 ; CN1CCC[C@H]1COc1nc2c(c(N3CC4CCC(C3)N4)n1)CCN(c1cncc3cccc(Cl)c13)C2 ; Cc1cc(N)nc(N2CCc3c(nc(OC[C@@H]4C[C@@H](F)CN4C)nc3N3CC4CCC(C3)N4)C2)c1C(F)(F)F ; C#Cc1cccc2cccc(N3CCc4c(nc(OC[C@@H]5CCCN5C)nc4N4CC5CCC(C4)N5)C3)c12]

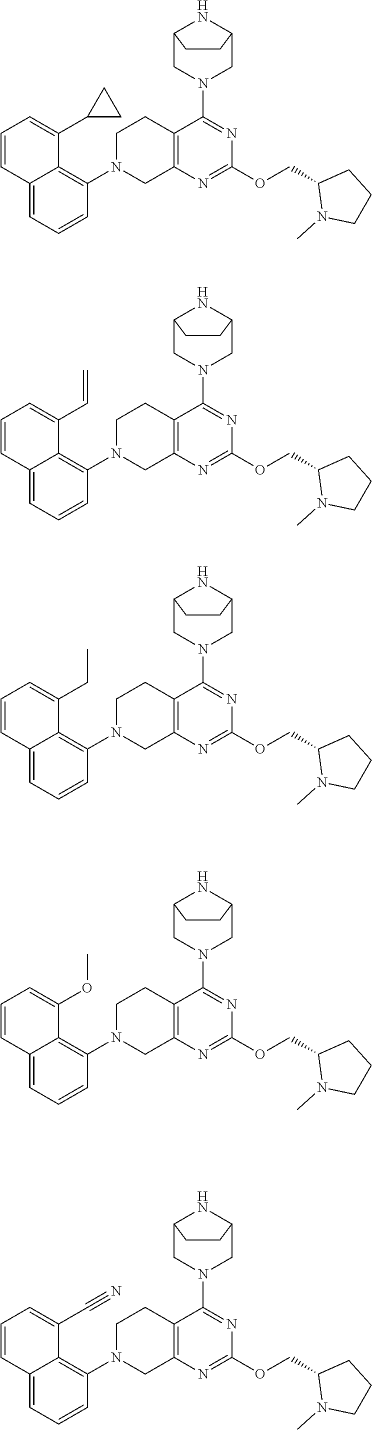 [SMILES: CN1CCC[C@H]1COc1nc2c(c(N3CC4CCC(C3)N4)n1)CCN(c1cccc3cccc(C4CC4)c13)C2 ; C=Cc1cccc2cccc(N3CCc4c(nc(OC[C@@H]5CCCN5C)nc4N4CC5CCC(C4)N5)C3)c12 ; CCc1cccc2cccc(N3CCc4c(nc(OC[C@@H]5CCCN5C)nc4N4CC5CCC(C4)N5)C3)c12 ; COc1cccc2cccc(N3CCc4c(nc(OC[C@@H]5CCCN5C)nc4N4CC5CCC(C4)N5)C3)c12 ; CN1CCC[C@H]1COc1nc2c(c(N3CC4CCC(C3)N4)n1)CCN(c1cccc3cccc(C#N)c13)C2]

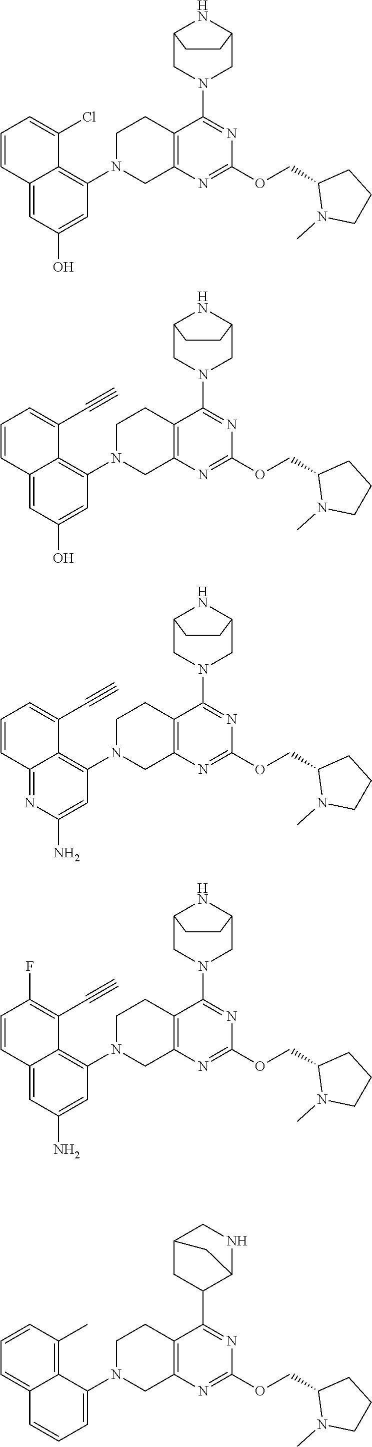 [SMILES: CN1CCC[C@H]1COc1nc2c(c(N3CC4CCC(C3)N4)n1)CCN(c1cc(O)cc3cccc(Cl)c13)C2 ; C#Cc1cccc2cc(O)cc(N3CCc4c(nc(OC[C@@H]5CCCN5C)nc4N4CC5CCC(C4)N5)C3)c12 ; C#Cc1cccc2nc(N)cc(N3CCc4c(nc(OC[C@@H]5CCCN5C)nc4N4CC5CCC(C4)N5)C3)c12 ; C#Cc1c(F)ccc2cc(N)cc(N3CCc4c(nc(OC[C@@H]5CCCN5C)nc4N4CC5CCC(C4)N5)C3)c12 ; Cc1cccc2cccc(N3CCc4c(nc(OC[C@@H]5CCCN5C)nc4C4CC5CNC4C5)C3)c12]

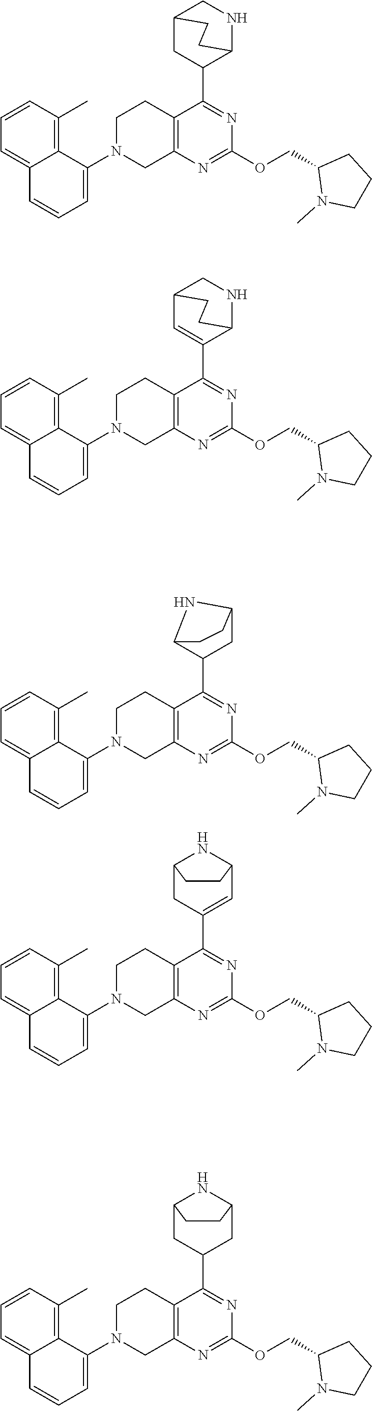 [SMILES: Cc1cccc2cccc(N3CCc4c(nc(OC[C@@H]5CCCN5C)nc4C4CC5CCC4NC5)C3)c12 ; Cc1cccc2cccc(N3CCc4c(nc(OC[C@@H]5CCCN5C)nc4C4=CC5CCC4NC5)C3)c12 ; Cc1cccc2cccc(N3CCc4c(nc(OC[C@@H]5CCCN5C)nc4C4CC5CCC4N5)C3)c12 ; Cc1cccc2cccc(N3CCc4c(nc(OC[C@@H]5CCCN5C)nc4C4=CC5CCC(C4)N5)C3)c12 ; Cc1cccc2cccc(N3CCc4c(nc(OC[C@@H]5CCCN5C)nc4C4CC5CCC(C4)N5)C3)c12]

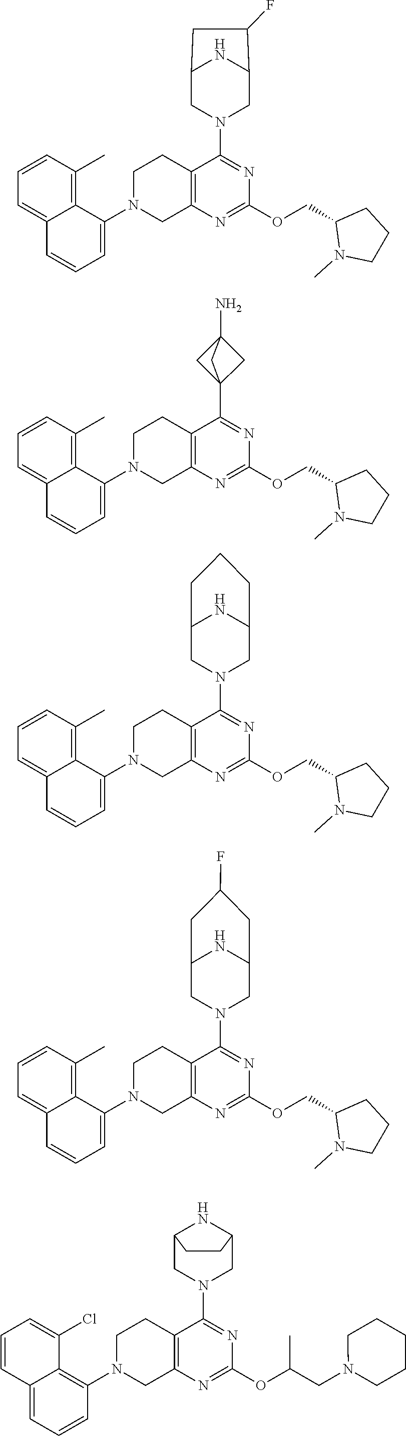 [SMILES: Cc1cccc2cccc(N3CCc4c(nc(OC[C@@H]5CCCN5C)nc4N4CC5CC(F)C(C4)N5)C3)c12 ; Cc1cccc2cccc(N3CCc4c(nc(OC[C@@H]5CCCN5C)nc4C45CC(N)(C4)C5)C3)c12 ; Cc1cccc2cccc(N3CCc4c(nc(OC[C@@H]5CCCN5C)nc4N4CC5CCCC(C4)N5)C3)c12 ; Cc1cccc2cccc(N3CCc4c(nc(OC[C@@H]5CCCN5C)nc4N4CC5CC(F)CC(C4)N5)C3)c12 ; CC(CN1CCCCC1)Oc1nc2c(c(N3CC4CCC(C3)N4)n1)CCN(c1cccc3cccc(Cl)c13)C2]

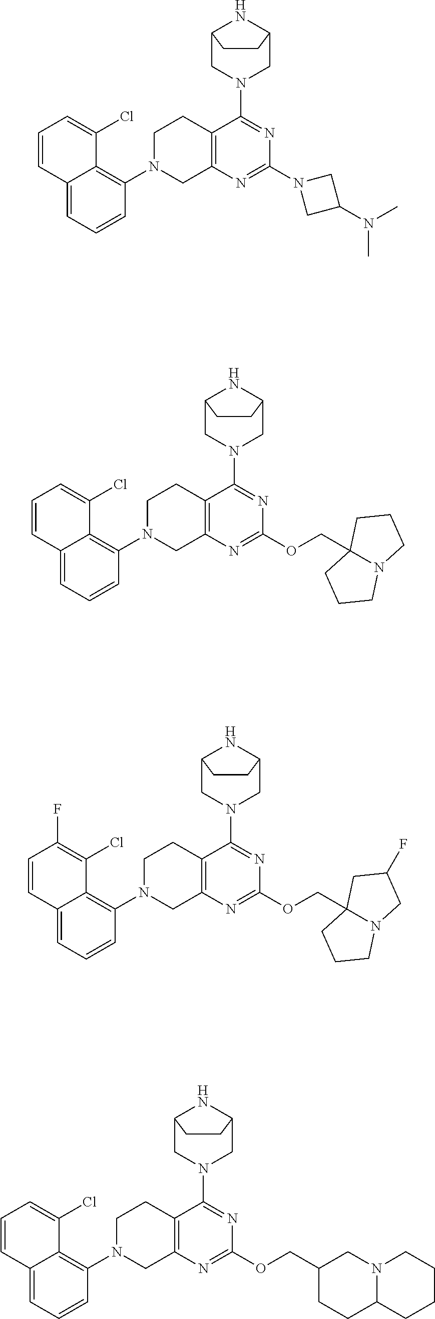 [SMILES: CN(C)C1CN(c2nc3c(c(N4CC5CCC(C4)N5)n2)CCN(c2cccc4cccc(Cl)c24)C3)C1 ; Clc1cccc2cccc(N3CCc4c(nc(OCC56CCCN5CCC6)nc4N4CC5CCC(C4)N5)C3)c12 ; Fc1ccc2cccc(N3CCc4c(nc(OCC56CCCN5CC(F)C6)nc4N4CC5CCC(C4)N5)C3)c2c1Cl ; Clc1cccc2cccc(N3CCc4c(nc(OCC5CCC6CCCCN6C5)nc4N4CC5CCC(C4)N5)C3)c12]

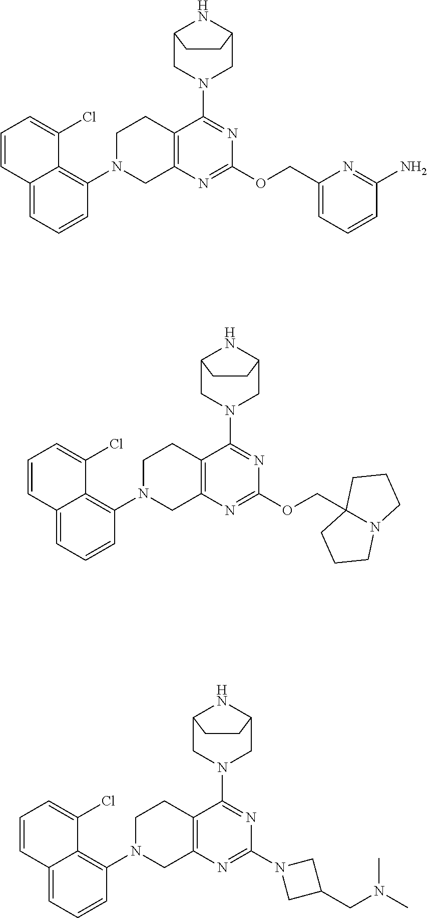 [SMILES: Nc1cccc(COc2nc3c(c(N4CC5CCC(C4)N5)n2)CCN(c2cccc4cccc(Cl)c24)C3)n1 ; Clc1cccc2cccc(N3CCc4c(nc(OCC56CCCN5CCC6)nc4N4CC5CCC(C4)N5)C3)c12 ; CN(C)CC1CN(c2nc3c(c(N4CC5CCC(C4)N5)n2)CCN(c2cccc4cccc(Cl)c24)C3)C1]

In the second aspect, disclosed herein is a pharmaceutical composition comprising the compound disclosed herein, or a pharmaceutically acceptable salt thereof, and at least one pharmaceutically acceptable carrier or excipient.

In the third aspect, disclosed herein is a method of inhibiting KRAS G12D activity, which comprises administering to an individual the compound disclosed herein, or a pharmaceutically acceptable salt thereof, including the compound of formula (I) or the specific compounds exemplified herein.

In the fourth aspect, disclosed herein is a method of treating a disease or disorder in a patient comprising administering to the patient a therapeutically effective amount of the compound disclosed herein, or a pharmaceutically acceptable salt thereof as a KRAS G12D inhibitor, wherein the compound disclosed herein includes the compound of formula (I) or the specific compounds exemplified herein. In some embodiments, the disease or disorder is associated with inhibition of KRAS G12D interaction. Preferably, the disease or disorder is cancer.

In the fifth aspect, disclosed herein is a bifunctional compound composed of a target protein (i.e., KRAS G12D)-binding moiety and an E3 ubiquitin ligase-binding moiety, which has been shown to induce proteasome-mediated degradation of selected proteins. In some embodiments, the bifunctional compound disclosed herein is composed of a target protein (i.e., KRAS G12D)-binding moiety disclosed herein and an E3 ubiquitin ligase-binding moiety known in the art. In some embodiments, disclosed herein is the use of the compound disclosed herein in the preparation of degrading a target protein compound by using chemical modification of the compound disclosed herein.

### DETAILED DESCRIPTION OF THE INVENTION

As used herein, the following words, phrases and symbols are generally intended to have the meanings as set forth below, except to the extent that the context in which they are used indicates otherwise.

The following abbreviations and terms have the indicated meanings throughout:

The phrase “a” or “an” entity as used herein refers to one or more of that entity. For example, a compound refers to one or more compounds or at least one compound. For another example, “. . . substituted with a substituent . . . ” means that one or more substituents are substituted as long as valence and stability permit. As such, the terms “a” (or “an”), “one or more”, and “at least one” can be used interchangeably herein.

The term “alkyl” herein refers to a hydrocarbon group selected from linear and branched saturated hydrocarbon groups comprising from 1 to 18, such as from 1 to 12, further such as from 1 to 10, more further such as from 1 to 8, or from 1 to 6, or from 1 to 4, carbon atoms. Examples of alkyl groups comprising from 1 to 6 carbon atoms (i.e., C₁₋₆ alkyl) include, but not limited to methyl, ethyl, 1-propyl or n-propyl (“n-Pr”), 2-propyl or isopropyl (“i-Pr”), 1-butyl or n-butyl (“n-Bu”), 2-methyl-1-propyl or isobutyl (“i-Bu”), 1-methylpropyl or s-butyl (“s-Bu”), 1,1-dimethylethyl ort-butyl (“t-Bu”), 1-pentyl, 2-pentyl, 3-pentyl, 2-methyl-2-butyl, 3-methyl-2-butyl, 3-methyl-1-butyl, 2-methyl-1-butyl, 1-hexyl, 2-hexyl, 3-hexyl, 2-methyl-2-pentyl, 3-methyl-2-pentyl, 4-methyl-2-pentyl, 3-methyl-3-pentyl, 2-methyl-3-pentyl, 2,3-dimethyl-2-butyl and 3,3-dimethyl-2-butyl groups.

The term “alkyloxy” herein refers to an alkyl group as defined above bonded to oxygen, represented by -Oalkyl. Examples of an alkyloxy, e.g., C₁₋₆ alkyloxy or C₁₋₄ alkyloxy includes, but not limited to, methoxy, ethoxyl, isopropoxy, propoxy, n-butoxy, tert-butoxy, pentoxy and hexoxy and the like.

The term “haloalkyl” herein refers to an alkyl group in which one or more hydrogen is/are replaced by one or more halogen atoms such as fluoro, chloro, bromo, and iodo. Examples of the haloalkyl include C₁₋₆haloalkyl or C₁₋₄haloalkyl, but not limited to F₃C—, ClCH₂—, CF₃CH₂—, CF₃CCl₂—, and the like.

The term “alkenyl” herein refers to a hydrocarbon group selected from linear and branched hydrocarbon groups comprising at least one C═C double bond and from 2 to 18, such as from 2 to 8, further such as from 2 to 6, carbon atoms. Examples of the alkenyl group, e.g., C₂₋₆ alkenyl, include, but not limited to ethenyl or vinyl, prop-1-enyl, prop-2-enyl, 2-methylprop-1-enyl, but-1-enyl, but-2-enyl, but-3-enyl, buta-1,3-dienyl, 2-methylbuta-1,3-dienyl, hex-1-enyl, hex-2-enyl, hex-3-enyl, hex-4-enyl, and hexa-1,3-dienyl groups.

The term “alkynyl” herein refers to a hydrocarbon group selected from linear and branched hydrocarbon group, comprising at least one C═C triple bond and from 2 to 18, such as 2 to 8, further such as from 2 to 6, carbon atoms. Examples of the alkynyl group, e.g., C₂₋₆ alkynyl, include, but not limited to ethynyl, 1-propynyl, 2-propynyl (propargyl), 1-butynyl, 2-butynyl, and 3-butynyl groups.

The term “cycloalkyl” herein refers to a hydrocarbon group selected from saturated and partially unsaturated cyclic hydrocarbon groups, comprising monocyclic and polycyclic (e.g., bicyclic and tricyclic) groups. For example, the cycloalkyl group may comprise from 3 to 12, such as from 3 to 10, further such as 3 to 8, further such as 3 to 6, 3 to 5, or 3 to 4 carbon atoms. Even further for example, the cycloalkyl group may be selected from monocyclic group comprising from 3 to 12, such as from 3 to 10, further such as 3 to 8, 3 to 6 carbon atoms. Examples of the monocyclic cycloalkyl group include cyclopropyl, cyclobutyl, cyclopentyl, 1-cyclopent-1-enyl, 1-cyclopent-2-enyl, 1-cyclopent-3-enyl, cyclohexyl, 1-cyclohex-1-enyl, 1-cyclohex-2-enyl, 1-cyclohex-3-enyl, cyclohexadienyl, cycloheptyl, cyclooctyl, cyclononyl, cyclodecyl, cycloundecyl, and cyclododecyl groups. In particular, Examples of the saturated monocyclic cycloalkyl group, e.g., C₃₋₈ cycloalkyl, include, but not limited to cyclopropyl, cyclobutyl, cyclopentyl, cyclohexyl, cycloheptyl, and cyclooctyl groups. Examples of the bicyclic cycloalkyl groups include those having from 7 to 12 ring atoms arranged as a bicyclic ring selected from [4, 4], [4, 5], [5, 5], [5, 6] and [6, 6] ring systems, or as a bridged bicyclic ring selected from bicyclo[2.2.1]heptane, bicyclo[2.2.2]octane, and bicyclo[3.2.2]nonane. Further Examples of the bicyclic cycloalkyl groups include those arranged as a bicyclic ring selected from [5, 6] and [6, 6] ring systems, such as

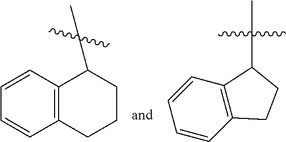 [SMILES: *C1CCCc2ccccc21 ; *C1CCc2ccccc21]

wherein the wavy lines indicate the points of attachment. The ring may be saturated or have at least one double bond (i.e. partially unsaturated), but is not fully conjugated, and is not aromatic, as aromatic is defined herein.

The term “bridged bicyclic ring” herein refers to a cyclic structure comprising two rings sharing three or more atoms, separating the two bridgehead atoms by a bridge containing at least one atom. In some embodiments, the bridged bicyclic ring may optionally comprise one or two double bonds in the ring structure. In some embodiments, the bridged bicyclic ring may independently comprise one or more, preferably one to two, heteroatoms independently selected from nitrogen, oxygen, and sulfur.

The term “cycloalkylene” refers to a divalent cyclopropyl as defined herein. For example, a cyclopropylene may be represented by

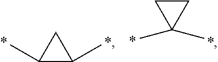

and so on, wherein asterisks refers to linking positions.

The suffix “diyl” refers to a divalent group. For example, oxetandiyl is a divalent group derived from oxetane, which may be represented by

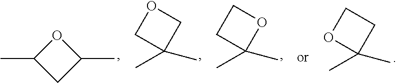

The term “aromatic ring” herein refers to an aromatic carbocyclic ring or aromatic heterocyclic ring (heteroaryl).

The term “aryl” and “aromatic carbocyclic ring” are used interchangeable throughout the disclosure herein, alone or in combination with other terms refers to a group selected from:
a. 5- and 6-membered monocyclic carbocyclic aromatic rings, for example, phenyl
b. bicyclic ring systems such as 7- to 12-membered bicyclic ring systems wherein at least one ring is carbocyclic and aromatic, selected, for example, from naphthyl (such as naphtha-1-yl or naphtha-2-yl), indenyl; and
c. tricyclic ring systems such as 10- to 15-membered tricyclic ring systems wherein at least one ring is carbocyclic and aromatic, for example, phenanthrenyl, fluorenyl.

In some embodiments, examples of a carbocyclic aromatic ring include, for example, but not limited to, phenyl, naphth-1-yl, naphth-2-yl, anthracenyl, phenanthrenyl rings, and the like. In some embodiments, the carbocyclic aromatic ring is a naphthalene ring (naphth-1-yl or naphth-2-yl) or phenyl ring. In some embodiments, the aromatic hydrocarbon ring is a naphthyl or phenyl ring.

The term “aromatic heterocyclic ring” or “heteroaryl” herein refers to a group selected from:
a. 5- to 7-membered aromatic, monocyclic rings comprising at least one heteroatom, for example, from 1 to 4, or, in some embodiments, from 1 to 3, heteroatoms, selected from nitrogen, oxygen and sulfur, with the remaining ring atoms being carbon;
b. 8- to 12-membered bicyclic rings comprising at least one heteroatom, for example, from 1 to 4, or, in some embodiments, from 1 to 3, or, in other embodiments, 1 or 2, heteroatoms, selected from nitrogen, oxygen and sulfur, with the remaining ring atoms being carbon and wherein at least one ring is aromatic and at least one heteroatom is present in the aromatic ring; and
c. 11- to 14-membered tricyclic rings comprising at least one heteroatom, for example, from 1 to 4, or in some embodiments, from 1 to 3, or, in other embodiments, 1 or 2, heteroatoms, selected from nitrogen, oxygen and sulfur, with the remaining ring atoms being carbon and wherein at least one ring is aromatic and at least one heteroatom is present in an aromatic ring.

When the total number of S and O atoms in the heteroaryl group exceeds 1, those heteroatoms are not adjacent to one another. In some embodiments, the total number of S and O atoms in the heteroaryl group is not more than 2. In some embodiments, the total number of S and O atoms in the aromatic heterocycle is not more than 1. When the heteroaryl group contains more than one heteroatom ring member, the heteroatoms may be the same or different. The nitrogen atoms in the ring(s) of the heteroaryl group can be oxidized to form N-oxides.

The terms “aromatic heterocyclic ring” and “heteroaryl” are used interchangeable throughout the disclosure herein. In some embodiments, a monocyclic or bicyclic aromatic heterocyclic ring has 5- to 10-ring forming members with 1, 2, 3, or 4 heteroatom ring members independently selected from nitrogen, sulfur and oxygen and the remaining ring members being carbon. In some embodiments, the monocyclic or bicyclic aromatic heterocyclic ring is a monocyclic or bicyclic ring comprising 1 or 2 heteroatom ring members independently selected from nitrogen, sulfur and oxygen. In some embodiments, the monocyclic or bicyclic aromatic heterocyclic ring is a 5- to 6-membered heteroaryl ring, which is monocyclic and which has 1 or 2 heteroatom ring members independently selected from nitrogen, sulfur and oxygen. In some embodiments, the monocyclic or bicyclic aromatic heterocyclic ring is a 8- to 10-membered heteroaryl ring, which is bicyclic and which has 1 or 2 heteroatom ring members independently selected from nitrogen, sulfur and oxygen.

Examples of the heteroaryl group or the monocyclic or bicyclic aromatic heterocyclic ring include, but are not limited to, (as numbered from the linkage position assigned priority 1) pyridyl (such as 2-pyridyl, 3-pyridyl, or 4-pyridyl), cinnolinyl, pyrazinyl, 2,4-pyrimidinyl, 3,5-pyrimidinyl, 2,4-imidazolyl, imidazopyridinyl, isoxazolyl, oxazolyl, thiazolyl, isothiazolyl, thiadiazolyl (such as 1,2,3-thiadiazolyl, 1,2,4-thiadiazolyl, or 1,3,4-thiadiazolyl), tetrazolyl, thienyl (such as thien-2-yl, thien-3-yl), triazinyl, benzothienyl, furyl or furanyl, benzofuryl, benzoimidazolyl, indolyl, isoindolyl, indolinyl, oxadiazolyl (such as 1,2,3-oxadiazolyl, 1,2,4-oxadiazolyl, or 1,3,4-oxadiazolyl), phthalazinyl, pyrazinyl, pyridazinyl, pyrrolyl, triazolyl (such as 1,2,3-triazolyl, 1,2,4-triazolyl, or 1,3,4-triazolyl), quinolinyl, isoquinolinyl, pyrazolyl, pyrrolopyridinyl (such as 1H-pyrrolo[2,3-b]pyridin-5-yl), pyrazolopyridinyl (such as 1H-pyrazolo[3,4-b]pyridin-5-yl), benzoxazolyl (such as benzo[d]oxazol-6-yl), pteridinyl, purinyl, 1-oxa-2,3-diazolyl, 1-oxa-2,4-diazolyl, 1-oxa-2,5-diazolyl, 1-oxa-3,4-diazolyl, 1-thia-2,3-diazolyl, 1-thia-2,4-diazolyl, 1-thia-2,5-diazolyl, 1-thia-3,4-diazolyl, furazanyl (such as furazan-2-yl, furazan-3-yl), benzofurazanyl, benzothiophenyl, benzothiazolyl, benzoxazolyl, quinazolinyl, quinoxalinyl, naphthyridinyl, furopyridinyl, benzothiazolyl (such as benzo[d]thiazol-6-yl), indazolyl (such as 1H-indazol-5-yl) and 5,6,7,8-tetrahydroisoquinoline.

The term “heterocyclic” or “heterocycle” or “heterocyclyl” herein refers to a ring selected from 4- to 12-membered monocyclic, bicyclic and tricyclic, saturated and partially unsaturated rings comprising at least one carbon atoms in addition to at least one heteroatom, such as from 1-4 heteroatoms, further such as from 1-3, or further such as 1 or 2 heteroatoms, selected from oxygen, sulfur, and nitrogen. In some embodiments, a heterocyclyl group is 4- to 7-membered monocyclic ring with one heteroatom selected from nitrogen, oxygen and sulfur. “Heterocycle” herein also refers to a 5- to 7-membered heterocyclic ring comprising at least one heteroatom selected from nitrogen, oxygen and sulfur fused with 5-, 6-, and/or 7-membered cycloalkyl, carbocyclic aromatic or heteroaromatic ring, provided that the point of attachment is at the heterocyclic ring when the heterocyclic ring is fused with a carbocyclic aromatic or a heteroaromatic ring, and that the point of attachment can be at the cycloalkyl or heterocyclic ring when the heterocyclic ring is fused with cycloalkyl. “Heterocycle” herein also refers to a 5- to 20-membered polycyclic heterocyclyl with rings connected through one common carbon atom (called a spiro atom), wherein said rings have one or more heteroatoms selected from nitrogen, oxygen or sulfur as the ring members, provided that the point of attachment is at the heterocyclic ring. The spiro rings may be saturated or have at least one double bond (i.e. partially unsaturated), but none of the rings has a completely conjugated pi-electron system. Preferably a spiro heterocyclyl is 6- to 14-membered, and more preferably 7- to 10-membered or 7- to 9-membered. According to the number of common spiro atoms, a spiro heterocyclyl is divided into mono-spiro heterocyclyl, di-spiro heterocyclyl, or poly-spiro heterocyclyl, and preferably refers to mono-spiro heterocyclyl or di-spiro heterocyclyl, and more preferably 4-membered/4-membered, 4-membered/5-membered, 4-membered/6-membered, 5-membered/5-membered, or 5-membered/6-membered mono-spiro heterocyclyl. Representative examples of spiro heterocyclyls include, but are not limited to the following groups, such as

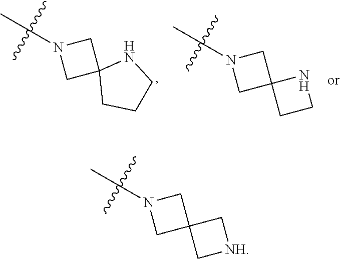 [SMILES: *N1CC2(CCCN2)C1 ; *N1CC2(CCN2)C1 ; *N1CC2(CNC2)C1]

The heterocycle may be substituted with alkyl or oxo. The point of the attachment may be carbon or heteroatom in the heterocyclic ring. A heterocycle is not a heteroaryl as defined herein.

Examples of the heterocycle include, but not limited to, (as numbered from the linkage position assigned priority 1) 1-pyrrolidinyl, 2-pyrrolidinyl, 2, 4-imidazolidinyl, 2, 3-pyrazolidinyl, 1-piperidinyl, 2-piperidinyl, 3-piperidinyl, 4-piperidinyl, 2,5-piperazinyl, pyranyl, 2-morpholinyl, 3-morpholinyl, oxiranyl, aziridinyl, thiiranyl, azetidinyl, oxetanyl, thietanyl, 1,2-dithietanyl, 1,3-dithietanyl, dihydropyridinyl, tetrahydropyridinyl, thiomorpholinyl, thioxanyl, piperazinyl, homopiperazinyl, homopiperidinyl, azepanyl, oxepanyl, thiepanyl, 1,4-oxathianyl, 1,4-dioxepanyl, 1, 4-oxathiepanyl, 1,4-oxaazepanyl, 1,4-dithiepanyl, 1,4-thiazepanyl and 1,4-diazepane 1,4-dithianyl, 1,4-azathianyl, oxazepinyl, diazepinyl, thiazepinyl, dihydrothienyl, dihydropyranyl, dihydrofuranyl, tetrahydrofuranyl, tetrahydrothienyl, tetrahydropyranyl, tetrahydrothiopyranyl, 1-pyrrolinyl, 2-pyrrolinyl, 3-pyrrolinyl, indolinyl, 2H-pyranyl, 4H-pyranyl, 1,4-dioxanyl, 1,3-dioxolanyl, pyrazolinyl, pyrazolidinyl, dithianyl, dithiolanyl, pyrazolidinyl, imidazolinyl, pyrimidinonyl, 1,1-dioxo-thiomorpholinyl, 3-azabicyco[3.1.0]hexanyl, 3-azabicyclo[4.1.0]heptanyl and azabicyclo[2.2.2]hexanyl. A substituted heterocycle also includes a ring system substituted with one or more oxo moieties, such as piperidinyl N-oxide, morpholinyl-N-oxide, 1-oxo-1-thiomorpholinyl and 1,1-dioxo-1-thiomorpholinyl.

The term “fused ring” herein refers to a polycyclic ring system, e.g., a bicyclic or tricyclic ring system, in which two rings share only two ring atoms and one bond in common. Examples of fused rings may comprise a fused bicyclic cycloalkyl ring such as those having from 7 to 12 ring atoms arranged as a bicyclic ring selected from [4, 4], [4, 5], [5, 5], [5, 6] and [6, 6] ring systems as mentioned above; a fused bicyclic aryl ring such as 7- to 12-membered bicyclic aryl ring systems as mentioned above, a fused tricyclic aryl ring such as 10- to 15-membered tricyclic aryl ring systems mentioned above; a fused bicyclic heteroaryl ring such as 8- to 12-membered bicyclic heteroaryl rings as mentioned above, a fused tricyclic heteroaryl ring such as 11- to 14-membered tricyclic heteroaryl rings as mentioned above; and a fused bicyclic or tricyclic heterocyclyl ring as mentioned above.

The term “halogen” or “halo” herein refers to F, Cl, Br or I.

Compounds disclosed herein may contain an asymmetric center and may thus exist as enantiomers. Where the compounds disclosed herein possess two or more asymmetric centers, they may additionally exist as diastereomers. Enantiomers and diastereomers fall within the broader class of stereoisomers. All such possible stereoisomers as substantially pure resolved enantiomers, racemic mixtures thereof, as well as mixtures of diastereomers are intended to be included. All stereoisomers of the compounds disclosed herein and/or pharmaceutically acceptable salts thereof are intended to be included. Unless specifically mentioned otherwise, reference to one isomer applies to any of the possible isomers. Whenever the isomeric composition is unspecified, all possible isomers are included.

The term “substantially pure” as used herein means that the target stereoisomer contains no more than 35%, such as no more than 30%, further such as no more than 25%, even further such as no more than 20%, by weight of any other stereoisomer(s). In some embodiments, the term “substantially pure” means that the target stereoisomer contains no more than 10%, for example, no more than 5%, such as no more than 1%, by weight of any other stereoisomer(s).

When compounds disclosed herein contain olefinic double bonds, unless specified otherwise, such double bonds are meant to include both E and Z geometric isomers.

Some of the compounds disclosed herein may exist with different points of attachment of hydrogen, referred to as tautomers. For example, compounds including carbonyl —CH₂C(O)— groups (keto forms) may undergo tautomerism to form hydroxyl —CH═C(OH)— groups (enol forms). Both keto and enol forms, individually as well as mixtures thereof, are also intended to be included where applicable.

It may be advantageous to separate reaction products from one another and/or from starting materials. The desired products of each step or series of steps is separated and/or purified (hereinafter separated) to the desired degree of homogeneity by the techniques common in the art. Typically such separations involve multiphase extraction, crystallization from a solvent or solvent mixture, distillation, sublimation, or chromatography. Chromatography can involve any number of methods including, for example: reverse-phase and normal phase; size exclusion; ion exchange; high, medium and low pressure liquid chromatography methods and apparatus; small scale analytical; simulated moving bed (“SMB”) and preparative thin or thick layer chromatography, as well as techniques of small scale thin layer and flash chromatography. One skilled in the art will apply techniques most likely to achieve the desired separation.

Diastereomeric mixtures can be separated into their individual diastereomers on the basis of their physical chemical differences by methods well known to those skilled in the art, such as by chromatography and/or fractional crystallization. Enantiomers can be separated by converting the enantiomeric mixture into a diastereomeric mixture by reaction with an appropriate optically active compound (e.g., chiral auxiliary such as a chiral alcohol or Mosher's acid chloride), separating the diastereomers and converting (e.g., hydrolyzing) the individual diastereoisomers to the corresponding pure enantiomers. Enantiomers can also be separated by use of a chiral HPLC column.

A single stereoisomer, e.g., a substantially pure enantiomer, may be obtained by resolution of the racemic mixture using a method such as formation of diastereomers using optically active resolving agents (Eliel, E. and Wilen, S. Stereochemistry of Organic Compounds. New York: John Wiley & Sons, Inc., 1994; Lochmuller, C. H., et al. “Chromatographic resolution of enantiomers: Selective review.” J. Chromatogr., 113(3) (1975): pp. 283-302). Racemic mixtures of chiral compounds of the invention can be separated and isolated by any suitable method, including: (1) formation of ionic, diastereomeric salts with chiral compounds and separation by fractional crystallization or other methods, (2) formation of diastereomeric compounds with chiral derivatizing reagents, separation of the diastereomers, and conversion to the pure stereoisomers, and (3) separation of the substantially pure or enriched stereoisomers directly under chiral conditions. See: Wainer, Irving W., Ed. Drug Stereochemistry: Analytical Methods and Pharmacology. New York: Marcel Dekker, Inc., 1993.

“Pharmaceutically acceptable salts” refers to those salts which are, within the scope of sound medical judgment, suitable for use in contact with the tissues of humans and lower animals without undue toxicity, irritation, allergic response and the like, and are commensurate with a reasonable benefit/risk ratio. A pharmaceutically acceptable salt may be prepared in situ during the final isolation and purification of the compounds disclosed herein, or separately by reacting the free base function with a suitable organic acid or by reacting the acidic group with a suitable base.

In addition, if a compound disclosed herein is obtained as an acid addition salt, the free base can be obtained by basifying a solution of the acid salt. Conversely, if the product is a free base, an addition salt, such as a pharmaceutically acceptable addition salt, may be produced by dissolving the free base in a suitable organic solvent and treating the solution with an acid, in accordance with conventional procedures for preparing acid addition salts from base compounds. Those skilled in the art will recognize various synthetic methodologies that may be used without undue experimentation to prepare non-toxic pharmaceutically acceptable addition salts.

As defined herein, “a pharmaceutically acceptable salt thereof” include salts of at least one compound of Formula (I), and salts of the stereoisomers of at least one compound of Formula (I), such as salts of enantiomers, and/or salts of diastereomers.

“Treating”, “treat” or “treatment” or “alleviation” refers to administering at least one compound and/or at least one stereoisomer thereof, and/or at least one pharmaceutically acceptable salt thereof disclosed herein to a subject in recognized need thereof that has, for example, cancer.

The term “effective amount” refers to an amount of at least one compound and/or at least one stereoisomer thereof, and/or at least one pharmaceutically acceptable salt thereof disclosed herein effective to “treat” as defined above, a disease or disorder in a subject.

The term “at least one substituent” disclosed herein includes, for example, from 1 to 4, such as from 1 to 3, further as 1 or 2, substituents, provided that valence and stability permit. For example, “at least one substituent R⁷” disclosed herein includes from 1 to 4, such as from 1 to 3, further as 1 or 2, substituents selected from the list of R⁷ as disclosed herein; and “at least one substituent R¹⁰” disclosed herein includes from 1 to 4, such as from 1 to 3, further as 1 or 2, substituents selected from the list of R¹⁰ as disclosed herein.

The compounds disclosed herein, and/or the pharmaceutically acceptable salts thereof, can be synthesized from commercially available starting materials taken together with the disclosure herein.

Compounds of Formula (I) and Formula (II) may be prepared by the exemplary processes described in the working Examples, as well as relevant published literature procedures that are used by one skilled in the art. Exemplary reagents and procedures for these reactions appear hereinafter and in the working Examples. Protection and de-protection in the processes below may be carried out by procedures generally known in the art (see, for example, Greene, T. W. et al., eds., Protecting Groups in Organic Synthesis, 3rd Edition, Wiley (1999)). General methods of organic synthesis and functional group transformations are found in: Trost, B. M. et al., eds., Comprehensive Organic Synthesis: Selectivity, Strategy & Efficiency in Modern Organic Chemistry, Pergamon Press, New York, NY (1991); March, J., Advanced Organic Reactions, Mechanisms, and Structure. 4th Edition, Wiley & Sons, New York, NY (1992); Katritzky, A. R. et al., eds., Comprehensive Organic Functional Groups Transformations, 1st Edition, Elsevier Science Inc., Tarrytown, NY (1995); Larock, R. C., Comprehensive Organic Transformations, VCH Publishers, Inc., New York, NY (1989), and references therein.

Compounds of the invention (I) may be prepared according to the following schemes utilizing chemical transformations familiar to anyone of ordinary proficiency in the art of organic/medicinal chemistry. References to many of these transformations can be found in March's Advanced Organic Chemistry Reactions, Mechanisms, and Structure, Fifth Edition by Michael B. Smith and Jerry March, Wiley-Interscience, New York, 2001, or other standard texts on the topic of synthetic organic chemistry.

### General Synthetic Schemes

The target compounds are synthesized according to general schemes A and B.

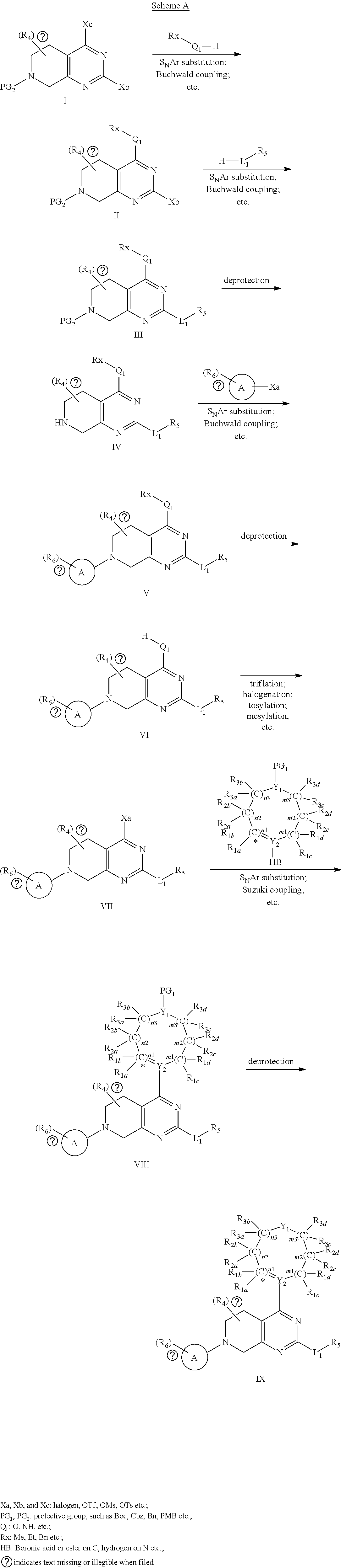

As shown in scheme A, a protective group was attached to intermediate I by S_{N}Ar substitution or Buchwald coupling etc. to give intermediate II. Then Xb was substituted by R₅-L1-H via S_{N}Ar substitution or Buchwald coupling etc. to give intermediate III, which was further deprotected to give intermediate IV. The following S_{N}Ar or Buchwald coupling etc. of intermediate IV with ring A derivatives gave intermediate V, which was further deprotected to give the intermediate VI. By triflation or halogenation etc., intermediate VI was converted into reactive intermediate VII for parallel synthesis. Intermediate VI was then coupled with top piece via S_{N}Ar substitution or Suzuki coupling to give intermediate VIII which was further deprotected to give final compound IX.

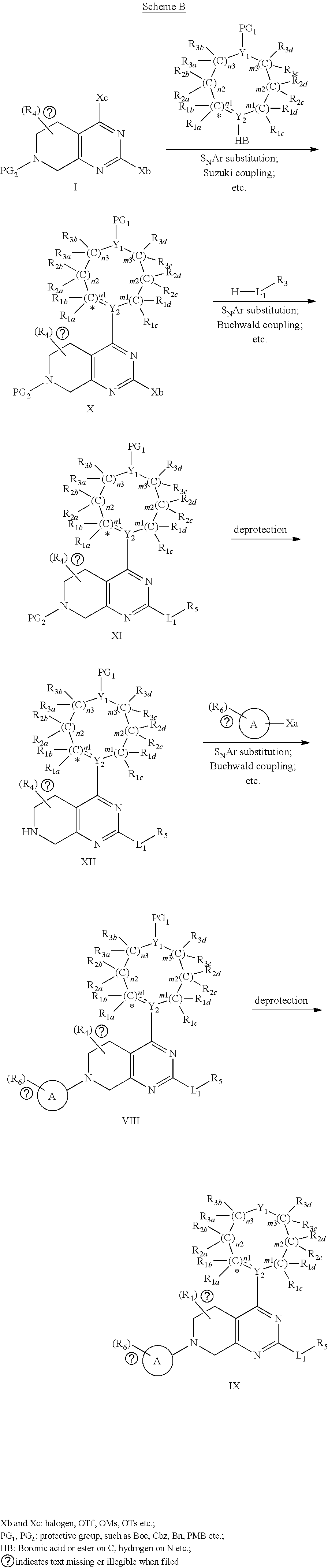

Scheme B is an alternative route for the target compounds, with similar reactions and slightly modified sequence. In this route, top piece was directly installed to the intermediate I and resulting product was used in the following steps by similar procedure as described in Scheme A.

### EXAMPLES

The Examples below are intended to be purely exemplary and should not be considered to be limiting in any way. Efforts have been made to ensure accuracy with respect to numbers used (for example, amounts, temperature, etc.), but some experimental errors and deviations should be accounted for. Unless indicated otherwise, temperature is in degrees Centigrade. Reagents were purchased from commercial suppliers such as Sigma-Aldrich, Alfa Aesar, or TCI, and were used without further purification unless otherwise indicated.

Unless otherwise indicated, the reactions set forth below were performed under a positive pressure of nitrogen or argon or with a drying tube in anhydrous solvents; the reaction flasks were fitted with rubber septa for the introduction of substrates and reagents via syringe; and glassware was oven dried and/or heat dried.

Unless otherwise indicated, column chromatography purification was conducted on a Biotage system (Manufacturer: Dyax Corporation) having a silica gel column or on a silica SepPak cartridge (Waters), or was conducted on a Teledyne Isco Combiflash purification system using prepacked silica gel cartridges.

¹H NMR spectra were recorded on a Varian instrument operating at 400 MHz or 500 MHz. ¹-NMR spectra were obtained using CDCl₃, CD₂Cl₂, CD₃OD, D₂O, d₆-DMSO, d₆-acetone or (CD₃)₂CO as solvent and tetramethylsilane (0.00 ppm) or residual solvent (CDClhd 3: 7.25 ppm; CD₃OD: 3.31 ppm; D₂O: 4.79 ppm; d₆-DMSO: 2.50 ppm; d₆-acetone: 2.05; (CD₃)₂CO: 2.05) as the reference standard. When peak multiplicities are reported, the following abbreviations are used: s (singlet), d (doublet), t (triplet), q (quartet), qn (quintuplet), sx (sextuplet), m (multiplet), br (broadened), dd (doublet of doublets), dt (doublet of triplets). Coupling constants, when given, are reported in Hertz (Hz). All compound names except the reagents were generated by ChemDraw version 18.0.

In the following Examples, the abbreviations below are used:
Ac Acetyl
ACN Acetonitrile
Aq Aqueous
BCA Bicinchoninic Acid
Brine Saturated aqueous sodium chloride solution
BINAP 2,2′-bis(diphenylphosphino)-1,1′-binaphthyl
Boc Tert-butyloxycarbonyl
BSA Bovine serum albumin
Cbz benzyloxycarbonyl
CH₂Cl₂ Dichloromethane
DMF N,N-Dimethylformamide
Dppf 1,1″-bis(diphenylphosphino)ferrocene
DCM Dichloromethane
DIEA or DIPEA N,N-diisopropylethylamine
DMAP 4-N,N-dimethylaminopyridine
DMF N,N-dimethylformamide
DMSO Dimethyl sulfoxide
DPBS Dulbecco's Phosphate Buffered Saline
EA or EtOAc Ethyl acetate
eq equivalent
ESI electrospray ionization
EtOH Ethanol
EtSH Ethanethiol
Et₂O or ether Diethyl ether
Et₃N Triethylamine
FA Formic acid
g Grams
GDP Guanosine diphosphate
GST Glutathione S-transferase
h or hr Hour
HCl Hydrochloric acid
HPLC High-performance liquid chromatography
mg Milligrams
mL Milliliters
Mmol Millimole
MeOH Methanol
Min Minutes
ms or MS Mass spectrum
Ms methanesulfonyl
NMR Nuclear magnetic resonance
Pd/C Palladium on carbon
PBS Phosphate buffered solution
Rt Retention time
Rt., RT. or rt. Room temperature
RT-FRET Real Time-Fluorescence Resonance Energy Transfer
Ru-Phos/Ru-PHOS 2-dicyclohexylphosphino-2′,6′-di-i-propoxy-1,1′-biphenyl
TBST Tris buffered saline with Tween 20
TEA Triethanolamine
TFA Trifluoroacetic acid
Tf2O Triflic anhydride
THF Tetrahydrofuran
μL Microliters
UV Ultraviolet
WT Wild type
Xantphos 4,5-Bis(diphenylphosphino)-9,9-dimethylxanthene

### Example 1: (S)-7-(8-methylnaphthalen-1-yl)-2-((1-methylpyrrolidin-2-yl)methoxy)-4-(piperazin-1-yl)-5,6,7,8-tetrahydropyrido[3,4-d]pyrimidine

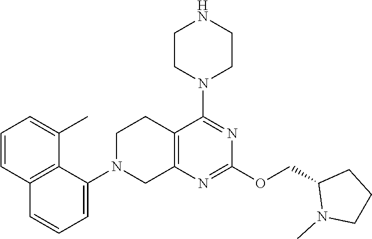 [SMILES: Cc1cccc2cccc(N3CCc4c(nc(OC[C@@H]5CCCN5C)nc4N4CCNCC4)C3)c12]

### Step 1: (S)-4-methoxy-7-(8-methylnaphthalen-1-yl)-2-((1-methylpyrrolidin-2-yl)methoxy)-5,6,7,8-tetrahydropyrido[3,4-d]pyrimidine

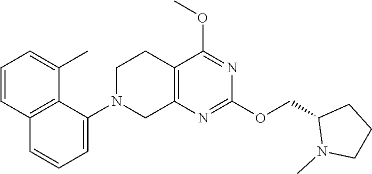 [SMILES: COc1nc(OC[C@@H]2CCCN2C)nc2c1CCN(c1cccc3cccc(C)c13)C2]

To a solution of (S)-4-methoxy-2-((l-methylpyrrolidin-2-yl)methoxy)-5,6,7,8-tetrahydropyrido[3,4-d]pyrimidine (30.0 g, 108 mmol) and 1-bromo-8-methylnaphthalene (26.2 g, 119 mmol) in toluene (300 mL) was added Xantphos (24.9 g, 43.1 mmol), Pd₂ (dba)₃ (19.7 g, 21.6 mmol) and Cs₂CO₃ (105 g, 323 mmol). The mixture was stirred at 100° C. for 2 hrs. Upon completion, the reaction mixture was poured into water (800 mL). Aqueous layer was extracted with EtOAc (500 mL×3). The combined organic layers were washed with brine and dried over Na₂SO₄. Solvents were evaporated and the residue was purified by column chromatography (DCM/MeOH=10:1) to give the title compound (23.0 g, 51%). MS (ESI, m/e) [M+1]⁺419.2.

### Step 2: (S)-7-(8-methylnaphthalen-1-yl)-2-((1-methylpyrrolidin-2-yl)methoxy)-5,6,7,8-tetrahydropyrido[3,4-d]pyrimidin-4-ol

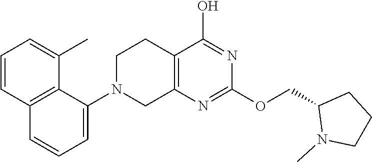 [SMILES: Cc1cccc2cccc(N3CCc4c(O)nc(OC[C@@H]5CCCN5C)nc4C3)c12]

To a solution of NaH (2.9 g, 71.7 mmol) in DMF (230 mL) was added EtSH (8.7 g, 140 mmol) at 0° C. The mixture was stirred at 25° C. for 30 mins followed by addition of (S)-4-methoxy-7-(8-methylnaphthalen-1-yl)-2-((1-methylpyrrolidin-2-yl)methoxy)-5,6,7,8-tetrahydropyrido[3,4-d]pyrimidine (15.0 g, 35.8 mmol). The reaction mixture was stirred at 60° C. for 1 h. Upon completion, the reaction mixture was diluted with cold water (1.00 L) and pH was adjusted to 7 with aqueous 1.00 M HCl. Aqueous layer was extracted with ethyl acetate/methanol=10:1 (800 mL x 4). The combined organic layers were concentrated and the residue was triturated with EtOAc (100 mL). The solid was dissolved in THF (2.00 L) and filtered, the filtrate was concentrated. The residue was triturated with MeOH (200 mL) to give the title compound (9.50 g, 65.1%). MS (ESI, m/e) [M+1]⁺405.2.

### Step 3: (S)-7-(8-methylnaphthalen-1-yl)-2-((1-methylpyrrolidin-2-yl)methoxy)-5,6,7,8-tetrahydropyrido[3,4-d]pyrimidin-4-yltrifluoromethanesulfonate

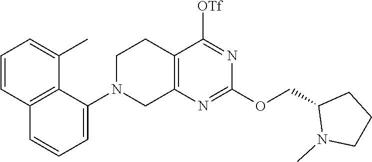 [SMILES: Cc1cccc2cccc(N3CCc4c(nc(OC[C@@H]5CCCN5C)nc4OS(=O)(=O)C(F)(F)F)C3)c12]

To a solution of (S)-7-(8-methylnaphthalen-1-yl)-2-((1-methylpyrrolidin-2-yl)methoxy)-5,6,7,8-tetrahydropyrido[3,4-d]pyrimidin-4-ol (500 mg, 1.24 mmol) and TEA (751 mg, 7.44 mmol) in DCM (40 mL) was added Tf₂O (1.05 g, 3.72 mmol) at 0° C. The reaction mixture was stirred at 0° C. for 1 h, H₂O (20 mL) was added. The aqueous layer was extracted with DCM (20 mL×2). The combined organic layers were washed with H₂ O (20 mL×2) and brine (20 mL). The solution was concentrated and used in the next step as crude. MS (ESI, m/e) [M+1]⁺537.4.

### Step 4: tert-butyl (S)-4-(7-(8-methylnaphthalen-1-yl)-2-((1-methylpyrrolidin-2-yl)methoxy)-5,6,7,8-tetrahydropyrido[3,4-d]pyrimidin-4-yl)piperazine-1-carboxylate

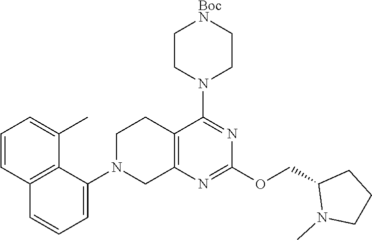 [SMILES: Cc1cccc2cccc(N3CCc4c(nc(OC[C@@H]5CCCN5C)nc4N4CCN(C(=O)OC(C)(C)C)CC4)C3)c12]

To a solution of (S)-7-(8-methylnaphthalen-1-yl)-2-((1-methylpyrrolidin-2-yl)methoxy)-5,6,7,8-tetrahydropyrido[3,4-d]pyrimidin-4-yltrifluoromethanesulfonate (100 mg, 0.186 mmol) and DIPEA (72 mg, 0.559 mmol) in CH₃CN (20 mL) was added tert-butyl piperazine-1-carboxylate (42 mg, 0.223 mmol), and the mixture was stirred at reflux overnight. Upon completion, solvent was removed and the crude product was purified by chromatography column on silica (eluting with DCM/MeOH=20/1) to give the title product (67 mg, 63% for 2 steps). MS (ESI, m/e) [M+1]⁺573.4.

### Step 5: (S)-7-(8-methylnaphthalen-1-yl)-2-((1-methylpyrrolidin-2-yl)methoxy)-4-(piperazin-1-yl)-5,6,7,8-tetrahydropyrido[3,4-d]pyrimidine

To a solution of tert-butyl (S)-4-(7-(8-methylnaphthalen-1-yl)-2-((1-methylpyrrolidin-2-yl)methoxy)-5,6,7,8-tetrahydropyrido[3,4-d]pyrimidin-4-yl)piperazine-1-carboxylate (67 mg, 0.117 mmol) in DCM (5 mL) was added TFA (5 mL), and the mixture was stirred at r.t for 2 h. Upon completion, solvent was removed and crude product was purified by prep-HPLC (ACN in water with 0.1% of FA, 0% to 90%) to give the title product (12 mg, 22% yield). ¹H NMR (400 MHz, CD₃OD) δ 7.71-7.65 (m, 2H), 7.43-7.40 (m, 1H), 7.34-73.0 (m, 2H), 7.26-7.24 (m, 1H), 4.69-4.48 (m, 2H), 4.13 (d, J=17.9 Hz, 1H), 3.91-3.82 (m, 2H), 3.75-3.71 (m, 4H), 3.63-3.54 (m, 2H), 3.42-3.35 (m, 2H), 3.19-3.13 (m, 4H), 2.98 (s, 3H), 2.91 (s, 3H), 2.71-2.62 (m, 1H), 2.40-2.28 (m, 1H), 2.18-1.98 (m, 3H). MS (ESI, m/e) [M+1]⁺473.4.

### Example 2: 7-(7-(8-methylnaphthalen-1-yl)-2-(((S)-1-methylpyrrolidin-2-yl)methoxy)-5,6,7,8-tetrahydropyrido[3,4-d]pyrimidin-4-yl)-7-azabicyclo[2.2.1]heptan-2-amine

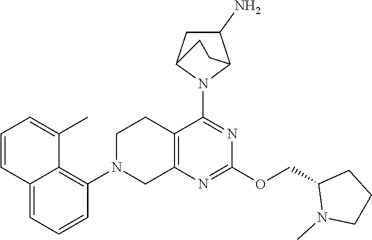 [SMILES: Cc1cccc2cccc(N3CCc4c(nc(OC[C@@H]5CCCN5C)nc4N4C5CCC4C(N)C5)C3)c12]

### Step 1: tert-butyl (7-(7-(8-methylnaphthalen-1-yl)-2-(((S)-1-methylpyrrolidin-2-yl)methoxy)-5,6,7,8-tetrahydropyrido[3,4-d]pyrimidin-4-yl)-7-azabicyclo[2.2.1]heptan-2-yl)carbamate

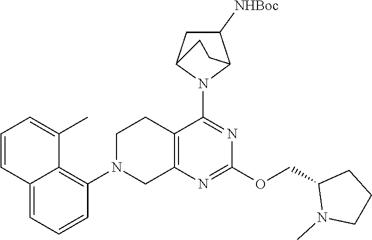 [SMILES: Cc1cccc2cccc(N3CCc4c(nc(OC[C@@H]5CCCN5C)nc4N4C5CCC4C(NC(=O)OC(C)(C)C)C5)C3)c12]

To a solution of (S)-7-(8-methylnaphthalen-1-yl)-2-((1-methylpyrrolidin-2-yl)methoxy)-5,6,7,8-tetrahydropyrido[3,4-d]pyrimidin-4-yltrifluoromethanesulfonate (150 mg, 0.28 mmol) in ACN (25 mL) was added tert-butyl (7-azabicyclo[2.2.1]heptan-2-yl)carbamate (83.1 mg, 0.392 mmol) and TEA (85 mg, 0.84 mmol) at room temperature, and the mixture was stirred at 70° C. for overnight. The resulting mixture was concentrated and crude product was purified by chromatography column on silica (eluting with DCM/MeOH=25/1) to give the title product (80 mg, 34%). MS (ESI, m/e) [M+1]⁺599.3.

### Step 2: 7-(7-(8-methylnaphthalen-1-yl)-2-(((S)-1-methylpyrrolidin-2-yl)methoxy)-5,6,7,8-tetrahydropyrido[3,4-d]pyrimidin-4-yl)-7-azabicyclo[2.2.1]heptan-2-amine

To a solution of tert-butyl (7-(7-(8-methylnaphthalen-1-yl)-2-(((S)-1-methylpyrrolidin-2-yl)methoxy)-5,6,7,8-tetrahydropyrido[3,4-d]pyrimidin-4-yl)-7-azabicyclo[2.2.1]heptan-2-yl)carbamate (80 mg, 0.13 mmol) in DCM (10 mL) was added TFA (2 mL), and the mixture was stirred at room temperature for 1 h. The resulting mixture was concentrated at room temperature and pH was adjusted to 7 with Na₂CO₃. The organic layer was concentrated to give a residue which was further purified by Prep-HPLC (ACN in water with 0.1% of FA, 0% to 90%) to give title product (3.48 mg, 5%). ¹H NMR (400 MHz, CD₃OD) δ 7.67 (dd, J=16.3, 8.1 Hz, 2H), 7.41 (t, J=7.7 Hz, 1H), 7.32 (t, J=7.6 Hz, 2H), 7.24 (d, J=6.7 Hz, 1H), 4.59 (d, J=11.9 Hz, 1H), 4.50-4.44 (m, 1H), 4.05 (d, J=17.7 Hz, 1H), 3.85 (m, 1H), 3.72 (d, J=17.4 Hz, 1H), 3.54 (m, 4H), 3.26-3.19 (m, 1H), 3.12 (m, 1H), 3.04-2.98 (m, 1H), 2.89 (s, 6H), 2.70 (m, 2H), 2.36-2.26 (m, 2H), 2.13-1.94 (m, 6H), 1.78 (dd, J=28.2, 9.9 Hz, 2H). MS (ESI, m/e) [M+1]⁺499.6.

### Example 3: 4-(3,8-diazabicyclo[3.2.1]octan-8-yl)-7-(8-chloronaphthalen-1-yl)-2-(((S)-methylpyrrolidin-2-yl)methoxy)-5,6,7,8-tetrahydropyrido[3,4-d]pyrimidine

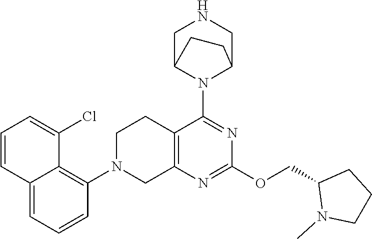 [SMILES: CN1CCC[C@H]1COc1nc2c(c(N3C4CCC3CNC4)n1)CCN(c1cccc3cccc(Cl)c13)C2]

Example 3 was prepared by similar procedure as described in Example 1 from (S)-7-(8-chloronaphthalen-1-yl)-2-((1-methylpyrrolidin-2-yl)methoxy)-5,6,7,8-tetrahydropyrido[3,4-d]pyrimidin-4-ol. ¹H NMR (400 MHz, CD₃OD) δ 7.84-7.80 (m, 1H), 7.70-7.68 (m, 1H), 7.53-7.48 (m, 2H), 7.39-7.33 (m, 2H), 4.95-4.93 (m, 2H), 4.77-4.75 (m, 1H), 4.66-4.63 (m, 1H), 4.62-4.49 (m, 1H), 4.30-4.26 (m, 1H), 3.75-3.71 (m, 2H), 3.63-3.52 (m, 2H), 3.32-3.12 (m, 5H), 3.00-2.98 (m, 3H), 2.68-2.65 (m, 1H), 2.36-2.28 (m, 2H), 2.18-1.89 (m, 6H). MS (ESI, m/e) [M+1]⁺519.4.

### Example 4: 4-(2,5-diazabicyclo[2.2.1]heptan-2-yl)-7-(8-methylnaphthalen-1-yl)-2-(((S)-1-methylpyrrolidin-2-yl)methoxy)-5,6,7,8-tetrahydropyrido[3,4-d]pyrimidine

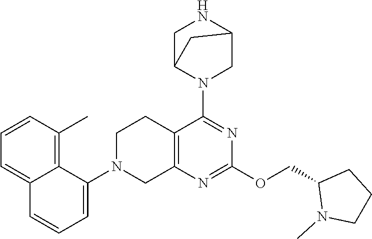 [SMILES: Cc1cccc2cccc(N3CCc4c(nc(OC[C@@H]5CCCN5C)nc4N4CC5CC4CN5)C3)c12]

Example 4 was prepared by similar procedure as described in Example 1 from tert-butyl 2,5-diazabicyclo[2.2.1]heptane-2-carboxylate. ¹H NMR (400 MHz, CD₃OD) δ 7.77-7.59 (m, 2H), 7.45-7.41 (m, 1H), 7.34-7.30 (m, 2H), 7.24-7.23 (m, 1H), 5.10 (s, 1H), 4.63-4.60 (m, 1H), 4.51-4.46 (m, 2H), 4.14-3.83 (m, 4H), 3.69-3.42 (m, 6H), 3.20-3.09 (m, 2H), 2.97-2.74(m, 7H), 2.35-2.20 (m, 2H), 2.09-1.96 (m, 4H). MS (ESI, m/e) [M+1]⁺485.4.

### Example 5: 4-(3,8-diazabicyclo[3.2.1]octan-3-yl)-7-(8-methylnaphthalen-1-yl)-2-(((S)-1-methylpyrrolidin-2-yl)methoxy)-5,6,7,8-tetrahydropyrido[3,4-d]pyrimidine

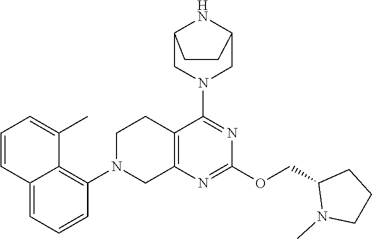 [SMILES: Cc1cccc2cccc(N3CCc4c(nc(OC[C@@H]5CCCN5C)nc4N4CC5CCC(C4)N5)C3)c12]

### Step 1: tert-butyl 3-(7-(8-methylnaphthalen-1-yl)-2-(((S)-1-methylpyrrolidin-2-yl)methoxy)-5,6,7,8-tetrahydropyrido[3,4-d]pyrimidin-4-yl)-3,8-diazabicyclo[3.2.1]octane-8-carboxylate

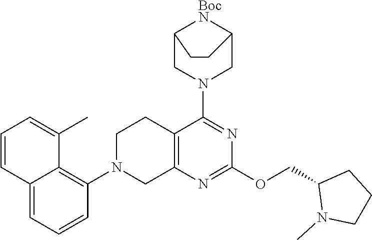 [SMILES: Cc1cccc2cccc(N3CCc4c(nc(OC[C@@H]5CCCN5C)nc4N4CC5CCC(C4)N5C(=O)OC(C)(C)C)C3)c12]

To a solution of (S)-7-(8-methylnaphthalen-1-yl)-2-((1-methylpyrrolidin-2-yl)methoxy)-5,6,7,8-tetrahydropyrido[3,4-d]pyrimidin-4-yltrifluoromethanesulfonate (175 mg, 0.326 mmol) in ACN (25 mL) was added tert-butyl 3,8-diazabicyclo[3.2.1]octane-8-carboxylate (104 mg, 0.489 mmol) and DIPEA (126.2 mg, 0.978 mmol) at room temperature, and the mixture was stirred at 70° C. for overnight. The resulting mixture was concentrated and the crude product was purified by chromatography column on silica (eluting with DCM/MeOH=25/1) to give the title product (114 mg, 58%). MS (ESI, m/e) [M+1]⁺599.3.

### Step 2: 4-(3,8-diazabicyclo[3.2.1]octan-3-yl)-7-(8-methylnaphthalen-1-yl)-2-(((S)-1-methylpyrrolidin-2-yl)methoxy)-5,6,7,8-tetrahydropyrido[3,4-d]pyrimidine

To a solution of tert-butyl 3-(7-(8-methylnaphthalen-1-yl)-2-(((S)-1-methylpyrrolidin-2-yl)methoxy)-5,6,7,8-tetrahydropyrido[3,4-d]pyrimidin-4-yl)-3,8-diazabicyclo[3.2.1]octane-8-carboxylate (114 mg, 0.19 mmol) in DCM (12 mL) was added TFA (3 mL) at room temperature, and the mixture was stirred at room temperature for 1 h. The resulting mixture was concentrated at room temperature and pH was adjusted to 7 with Na₂CO₃. The organic layer was concentrated to give a residue which was further purified by Prep-HPLC (ACN in water with 0.1% of FA, 0% to 90%) to give title product (32 mg, 33%). ¹H NMR (400 MHz, CD₃OD) δ 7.68 (dd, J=15.9, 8.0 Hz, 2H), 7.41 (t, J=7.7 Hz, 1H), 7.35-7.27 (m, 2H), 7.24 (d, J=6.9 Hz, 1H), 4.64-4.55 (m, 1H), 4.52-4.44 (m, 1H), 4.38 (d, J=13.5 Hz, 1H), 4.11 (d, J=18.0 Hz, 3H), 3.95 (d, J=13.9 Hz, 1H), 3.73 (d, J=18.0 Hz, 1H), 3.56 (m,4H), 3.23 (m, 3H), 3.03 (m, 1H), 2.91 (d, J=1.6 Hz, 6H), 2.67 (d, J=12.9 Hz, 1H), 2.34-2.23 (m, 2H), 2.17-1.99 (m, 5H), 1.98-1.87 (m, 1H). MS (ESI, m/e) [M+1]⁺499.6.

### Example 6: 2-(7-(8-methylnaphthalen-1-yl)-2-(((S)-1-methylpyrrolidin-2-yl)methoxy)-5,6,7,8-tetrahydropyrido[3,4-d]pyrimidin-4-yl)-2,5-diazabicyclo[2.2.2]octane

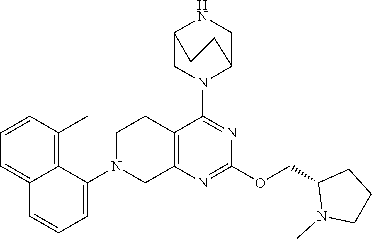 [SMILES: Cc1cccc2cccc(N3CCc4c(nc(OC[C@@H]5CCCN5C)nc4N4CC5CCC4CN5)C3)c12]

Example 6 was prepared by similar procedure as described in Example 1 from tert-butyl 2,5-diazabicyclo[2.2.2]octane-2-carboxylate. ¹H NMR (400 MHz, CD₃OD) δ 7.67 (dd, J=16.2, 8.1 Hz, 2H), 7.41 (t, J=7.7 Hz, 1H), 7.34-7.30 (t, J=7.6 Hz, 2H), 7.25-7.23 (m, 1H), 4.73-4.53 (m, 3H), 4.05-3.95 (m, 2H), 3.94-3.78 (m, 4H), 3.75-7.65 (m, 3H), 3.58-3.48 (m, 1H), 3.25-3.15 (m, 3H), 3.03 (s, 3H), 2.91-2.89 (m, 3H), 2.77 (d, J=12.7 Hz, 1H), 2.44-2.31 (m, 2H), 2.23-1.93 (m, 6H). MS (ESI, m/e) [M+1]⁺499.4.

### Example 7: 4-(3,8-diazabicyclo[3.2.1]octan-3-yl)-7-(8-chloronaphthalen-1-yl)-2-(((S)-1-methylpyrrolidin-2-yl)methoxy)-5,6,7,8-tetrahydropyrido[3,4-d]pyrimidine

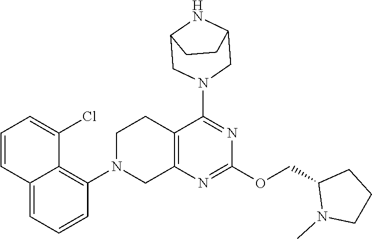 [SMILES: CN1CCC[C@H]1COc1nc2c(c(N3CC4CCC(C3)N4)n1)CCN(c1cccc3cccc(Cl)c13)C2]

### Step 1: benzyl 4-(8-(tert-butoxycarbonyl)-3,8-diazabicyclo[3.2.1]octan-3-yl)-2-chloro-5,8-dihydropyrido[3,4-d]pyrimidine-7(6H)-carboxylate

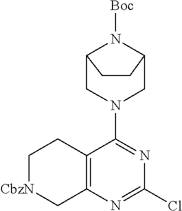 [SMILES: CC(C)(C)OC(=O)N1C2CCC1CN(c1nc(Cl)nc3c1CCN(C(=O)OCc1ccccc1)C3)C2]

To a solution of benzyl 2,4-dichloro-5,8-dihydropyrido[3,4-d]pyrimidine-7(6H)-carboxylate (10 g, 29.6 mmol) and DIPEA (13 mL, 74 mmol) in THF (50 mL) was added tert-butyl -3,8-diazabicyclo[3.2.1]octane-8-carboxylate (6.9 g, 32.56 mmol) at −20° C., and the mixture was stirred at −20° C. to r.t. for 1 day. Upon completion, solvent was removed and crude product was purified by chromatography column on silica (eluting with DCM/MeOH=40/1) to give the title product (12.3 g, 81%). MS (ESI, m/e) [M+1]⁺514.4.

### Step 2: benzyl 4-(8-(tert-butoxycarbonyl)-3,8-diazabicyclo[3.2.1]octan-3-yl)-2-(((S)-1-methylpyrrolidin-2-yl)methoxy)-5,8-dihydropyrido[3,4-d]pyrimidine-7(6H)-carboxylate

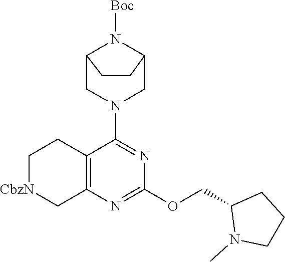 [SMILES: CN1CCC[C@H]1COc1nc2c(c(N3CC4CCC(C3)N4C(=O)OC(C)(C)C)n1)CCN(C(=O)OCc1ccccc1)C2]

A solution of benzyl 4-(8-(tert-butoxycarbonyl)-3,8-diazabicyclo[3.2.1]octan-3-yl)-2-chloro-5,8-dihydropyrido[3,4-d]pyrimidine-7(6H)-carboxylate (3 g, 5.84 mmol), (S)-(1-methylpyrrolidin-2-yl)methanol (2.0 g, 17.51 mmol), Pd₂(dba)₃ (535 mg, 0.584 mmol), RuPhos (540 mg, 1.168 mmol) and Cs₂CO₃ (4.75 g, 14.6 mmol) in toluene (50 mL) was stirred at 85° C. overnight. Upon completion, solvent was removed and crude product was purified by chromatography column on silica (eluting with DCM/MeOH=20/1) to give the title product (2.1 g, 61% yield). MS (ESI, m/e) [M+1]⁺593.4.

### Step 3: tert-butyl 3-(2-(((S)-1-methylpyrrolidin-2-yl)methoxy)-5,6,7,8-tetrahydropyrido[3,4-d]pyrimidin-4-yl)-3,8-diazabicyclo[3.2.1]octane-8-carboxylate

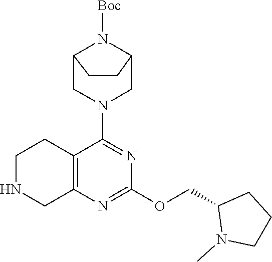 [SMILES: CN1CCC[C@H]1COc1nc2c(c(N3CC4CCC(C3)N4C(=O)OC(C)(C)C)n1)CCNC2]

To a solution of benzyl 4-(8-(tert-butoxycarbonyl)-3,8-diazabicyclo[3.2.1]octan-3-yl)-2-(((S)-1-methylpyrrolidin-2-yl)methoxy)-5,8-dihydropyrido[3,4-d]pyrimidine-7(6H)-carboxylate (1.6 g, 2.70 mmol) in methanol (10 mL) was added 10% Pd/C (300 mg), and the mixture was stirred at room temperature for 15 hrs. Then it was filtered and the filtrate was evaporated to give the title product (1.05 g, 85%). MS (ESI, m/e) [M+1]⁺459.4.

### Step 4: tert-butyl 3-(7-(8-chloronaphthalen-1-yl)-2-(((S)-1-methylpyrrolidin-2-yl)methoxy)-5,6,7,8-tetrahydropyrido[3,4-d]pyrimidin-4-yl)-3,8-diazabicyclo[3.2.1]octane-8-carboxylate

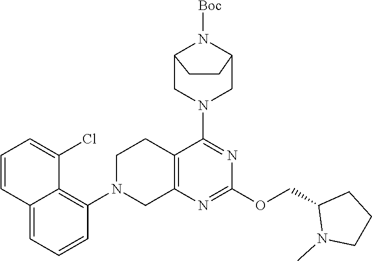 [SMILES: CN1CCC[C@H]1COc1nc2c(c(N3CC4CCC(C3)N4C(=O)OC(C)(C)C)n1)CCN(c1cccc3cccc(Cl)c13)C2]

A solution of tert-butyl 3-(2-(((S)-1-methylpyrrolidin-2-yl)methoxy)-5,6,7,8-tetrahydropyrido[3,4-d]pyrimidin-4-yl)-3,8-diazabicyclo[3.2.1]octane-8-carboxylate (200 mg, 0.365 mmol), 1-bromo-8-chloronaphthalene (106 mg, 0.438 mmol), Pd₂(dba)₃ (33 mg, 0.036mmol), RuPhos (34 mg, 0.073 mmol) and Cs₂CO₃ (297 mg, 0.91 mmol) in toluene (50 mL) was stirred at 85° C. overnight. Upon completion, solvent was removed and crude product was purified by chromatography column on silica (eluting with DCM/MeOH=20/1) to give the title product (116 mg, 53%). MS (ESI, m/e) [M+1]⁺619.4.

### Step 5: 4-(3,8-diazabicyclo[3.2.1]octan-3-yl)-7-(8-chloronaphthalen-1-yl)-2-(((S)-1-methylpyrrolidin-2-yl)methoxy)-5,6,7,8-tetrahydropyrido[3,4-d]pyrimidine

To a solution of tert-butyl 3-(7-(8-chloronaphthalen-1-yl)-2-(((S)-1-methylpyrrolidin-2-yl)methoxy)-5,6,7,8-tetrahydropyrido[3,4-d]pyrimidin-4-yl)-3,8-diazabicyclo[3.2.1]octane-8-carboxylate (116 mg, 0.194 mmol) in DCM (5 mL) was added TFA (5 mL), and the mixture was stirred at room temperature for 2 hrs. Upon completion, solvent was removed and crude product was purified by prep-HPLC to give the title product (25 mg, 26%). ¹H NMR (400 MHz, CD₃OD) δ 7.85-7.80 (m, 1H), 7.73-7.70 (m, 1H), 7.52-7.48 (m, 2H), 7.38-7.30 (m, 2H), 4.32-4.26 (m, 4H), 376-3.74 (m, 1H), 3.67-3.64 (m,1H), 3.58-3.56 (m, 3H), 3.37-3.75 (m, 1H), 3.15-3.04 (m, 4H), 2.79-2.72 (m, 1H), 2.57-2.55 (m, 1H), 2.50 (s, 3H), 2.48-2.40 (m, 1H), 2.12-2.02 (m, 2H). 1.84-1.68 (m, 6H). MS (ESI, m/e) [M+1]⁺519.4.

### Example 8: 3-(7-(8-methylnaphthalen-1-yl)-2-(((S)-1-methylpyrrolidin-2-yl)methoxy)-5,6,7,8-tetrahydropyrido[3,4-d]pyrimidin-4-yl)-3,6-diazabicyclo[3.1.1]heptane

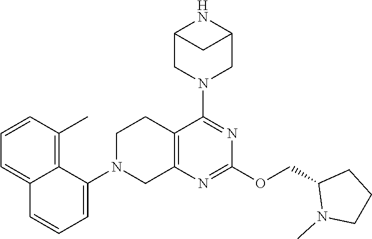 [SMILES: Cc1cccc2cccc(N3CCc4c(nc(OC[C@@H]5CCCN5C)nc4N4CC5CC(C4)N5)C3)c12]

### Step 1: tert-butyl 3-(7-(8-methylnaphthalen-1-yl)-2-(((S)-1-methylpyrrolidin-2-yl)methoxy)-5,6,7,8-tetrahydropyrido[3,4-d]pyrimidin-4-yl)-3,6-diazabicyclo[3.1.1]heptane-6-carboxylate

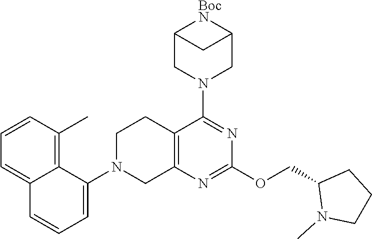 [SMILES: Cc1cccc2cccc(N3CCc4c(nc(OC[C@@H]5CCCN5C)nc4N4CC5CC(C4)N5C(=O)OC(C)(C)C)C3)c12]

To a solution of (S)-7-(8-methylnaphthalen-1-yl)-2-((1-methylpyrrolidin-2-yl)methoxy)-5,6,7,8-tetrahydropyrido[3,4-d]pyrimidin-4-yltrifluoromethanesulfonate (100 mg, 0.186 mmol) in MeCN (19 mL) was added tert-butyl 3,6-diazabicyclo[3.1.1]heptane-6-carboxylate (40 mg, 0.186 mmol) and DIPEA (0.2 mL, 1.15 mmol) at room temperature. The mixture was stirred at 70° C. for overnight. Then, the solvent was evaporated in vacuo and the residue was purified by chromatography (DCM to DCM/MeOH=10/1) to give the crude product (160 mg) used directly in the next step.

### Step 2: 3-(7-(8-methylnaphthalen-1-yl)-2-(((S)-1-methylpyrrolidin-2-yl)methoxy)-5,6,7,8-tetrahydropyrido[3,4-d]pyrimidin-4-yl)-3,6-diazabicyclo[3.1.1]heptane

To a solution of tert-butyl 3-(7-(8-methylnaphthalen-1-yl)-2-(((S)-1-methylpyrrolidin-2-y1)methoxy)-5,6,7,8-tetrahydropyrido[3,4-d]pyrimidin-4-yl)-3,6-diazabicyclo[3.1.1]heptane-6-carboxylate (160 mg, 0.274 mmol) in DCM (20 mL) was added 4M HCl in dioxane (4 mL). The mixture was stirred at room temperature for about 25 hrs. Then, the mixture was evaporated in vacuo. The residue was purified by Prep-HPLC (ACN in water with 0.1% of FA, 0% to 90%) to give the product (2.81 mg, 3% for 2 steps). ¹H NMR (400 MHz, CD₃OD) δ 8.52 (s, 1H), 6 7.67 (dd, J=21.8, 8.0 Hz, 2H), 7.42 (t, J=7.7 Hz, 1H), 7.35-7.28 (m, 2H), 7.24 (d, J=6.9 Hz, 1H), 4.63 (dd, J=12.9, 3.7 Hz, 1H), 4.59-4.36 (m, 3H), 4.31-4.16 (m, 3H), 4.10-3.98 (m, 2H), 3.71 (d, J=17.4 Hz, 1H), 3.60-3.51 (m, 1H), 3.47-3.34 (m, 3H), 3.23-3.13 (m, 1H), 3.05 (d, J=15.1 Hz, 1H), 2.97-2.92 (m, 1H), 2.90 (s, 3H), 2.87-2.82 (m, 1H), 2.80 (s, 3H), 2.25 (dt, J=15.5, 8.3 Hz, 1H), 2.04 (s, 1H), 2.03-1.97 (m, 1H), 1.94 (d, J=10.1 Hz, 1H), 1.86 (dt, J=13.7, 8.3 Hz, 1H). MS (ESI, m/e) [M+1]⁺485.4.

### Example 9: 4-(3,8-diazabicyclo[3.2.1]octan-3-yl)-7-(7,8-dichloronaphthalen-1-yl)-2-0(S)-1-methylpyrrolidin-2-yl)methoxy)-5,6,7,8-tetrahydropyrido[3,4-d]-dipyrimidine

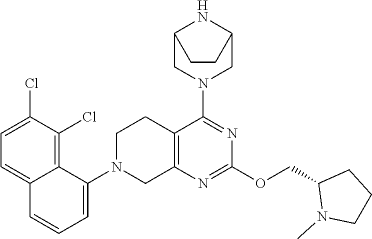 [SMILES: CN1CCC[C@H]1COc1nc2c(c(N3CC4CCC(C3)N4)n1)CCN(c1cccc3ccc(Cl)c(Cl)c13)C2]

Example 9 was prepared by similar procedure as described in Example 11 from 7,8-dichloronaphthalen-1-ol. ¹H NMR (400 MHz, CD₃OD) δ 7.87-7.76 (m, 1H), 7.75-7.61 (m, 1H), 7.65-7.48 (m, 2H), 7.46-7.31 (m,1H), 4.69-4.54 (m, 1H), 4.53-4.43 (m, 1H), 4.43-4.36 (m, 1H), 4.33-4.16 (m, 1H), 4.14-3.99 (m, 2H), 3.99-3.88 (m, 1H), 3.83-3.70 (m, 1H), 3.69-3.46 (m, 4H), 3.31-3.09 (m, 3H), 3.12-2.98 (m, 1H), 2.97-2.85 (m, 3H), 2.77-2.55 (m, 1H), 2.38-2.17 (m, 2H), 2.17-1.88 (m, 6H). MS (ESI, m/e) [M+1]⁺553.4.

### Example 10: 4-(4-(3,8-diazabicyclo[3.2.1]octan-3-yl)-2-0(S)-1-methylpyrrolidin-2-yl)methoxy)-5,8-dihydropyrido[3,4-d]pyrimidin-7(6H)-yl)naphthalen-2-ol

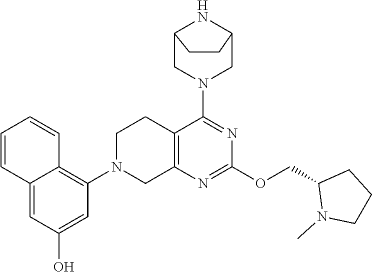 [SMILES: CN1CCC[C@H]1COc1nc2c(c(N3CC4CCC(C3)N4)n1)CCN(c1cc(O)cc3ccccc13)C2]

Example 10 was prepared by similar procedure as described in Example 7 from 4-bromonaphthalen-2-ol. ¹H NMR (400 MHz, CD₃OD) δ 8.06 (d, J=8.5 Hz, 1H), 7.63 (d, J=8.2 Hz, 1H), 7.37 (t, J=7.4 Hz, 1H), 7.26 (t, J=7.6 Hz, 1H), 6.88 (s, 1H), 6.79 (s, 1H), 4.63-4.60 (m, 1H), 4.51-4.47 (m, 1H), 4.42-4.18 (m, 4H), 4.11-4.02 (m, 2H), 3.62-3.53 (m, 3H), 3.44-3.41 (m, 3H), 3.10-2.84 (m, 7H), 2.42-2.26 (m, 2H), 2.26-1.87 (m, 6H). MS (ESI, m/e) [M+1]⁺501.4.

### Example 11: 4-(3,8-diazabicyclo[3.2.1]octan-3-yl)-7-(8-chloro-7-fluoronaphthalen-1-yl)-2-0(S)-1-methylpyrrolidin-2-yl)methoxy)-5,6,7,8-tetrahydropyrido[3,4-d]pyrimidine

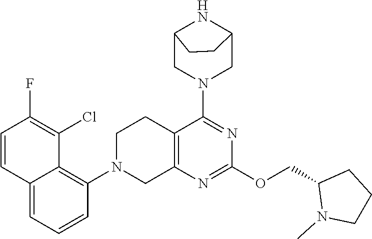 [SMILES: CN1CCC[C@H]1COc1nc2c(c(N3CC4CCC(C3)N4)n1)CCN(c1cccc3ccc(F)c(Cl)c13)C2]

### Step 1: 8-chloro-7-fluoronaphthalen-1-yl trifluoromethanesulfonate

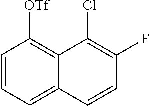 [SMILES: O=S(=O)(Oc1cccc2ccc(F)c(Cl)c12)C(F)(F)F]

To a solution of 8-chloro-7-fluoronaphthalen-1-ol (70 mg, 0.357 mmol) in DCM (15 mL) was added Tf₂O (161 mg, 0.571 mmol) and DIPEA (230 mg, 1.785 mmol) at 0° C., and the mixture was stirred at 0° C. for 0.5 h. The mixture was concentrated to get crude product which was used in the next step without further purification. MS (ESI, m/e) [M+1]⁺328.9.

### Step 2: tert-butyl 3-(7-(8-chloro-7-fluoronaphthalen-1-yl)-2-(((S)-1-methylpyrrolidin-2-yl)methoxy)-5,6,7,8-tetrahydropyrido[3,4-d]pyrimidin-4-yl)-3,8-diazabicyclo[3.2.1]octane-8-carboxylate

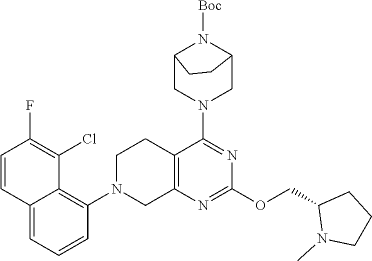 [SMILES: CN1CCC[C@H]1COc1nc2c(c(N3CC4CCC(C3)N4C(=O)OC(C)(C)C)n1)CCN(c1cccc3ccc(F)c(Cl)c13)C2]

To a solution of tert-butyl 3-(2-(((S)-1-methylpyrrolidin-2-yl)methoxy)-5,6,7,8-tetrahydropyrido[3,4-d]pyrimidin-4-yl)-3,8-diazabicyclo[3.2.1]octane-8-carboxylate (114 mg, 0.25 mmol) in toluene (15 mL) was added 8-chloro-7-fluoronaphthalen-1-yltrifluoromethanesulfonate (117.5 mg, 0.357 mmol), xantphos (41 mg, 0.0714 mmol), xantphos Pd G₃ (34 mg, 0.0357 mmol) and Cs₂CO₃ (291 mg, 0.89 mmol) at room temperature, and the mixture was stirred at 100° C. for overnight. The mixture was concentrated and the crude product was purified by chromatography column on silica (eluting with DCM/MeOH=15/1) to give the title product (60 mg, 26%). MS (ESI, m/e) [M+1]⁺637.3.

### Step 3: 4-(3,8-diazabicyclo[3.2.1]octan-3-yl)-7-(8-chloro-7-fluoronaphthalen-1-yl)-2-(((S)-1-methylpyrrolidin-2-yl)methoxy)-5,6,7,8-tetrahydropyrido[3,4-d]pyrimidine

To a solution of tert-butyl 3-(7-(8-chloro-7-fluoronaphthalen-1-yl)-2-(((S)-1-methylpyrrolidin-2-yl)methoxy)-5,6,7,8-tetrahydropyrido[3,4-d]pyrimidin-4-yl)-3,8-diazabicyclo[3.2.1]octane-8-carboxylate (60 mg, 0.09 mmol) in DCM (10 mL) was added TFA (3 mL), and the mixture was stirred at room temperature for 1 h. The resulting mixture was concentrated at room temperature and pH was adjusted to 7 with Na₂CO₃. The organic layer was concentrated to give a residue which was further purified by Prep-HPLC (ACN in water with 0.1% of FA, 0% to 90%) to give title product (20 mg, 40%). ¹H NMR (400 MHz, CD₃OD) δ 7.89 (dd, J=8.2, 6.0 Hz, 1H), 7.71 (d, J=8.1 Hz, 1H), 7.49 (t, J=7.8 Hz, 1H), 7.40 (t, J=8.2 Hz, 2H), 4.52 (td, J=11.5, 4.0 Hz, 1H), 4.48-4.41 (m, 1H), 4.36 (d, J=13.4 Hz, 1H), 4.28 (d, J=17.5 Hz, 1H), 4.00 (s, 2H), 3.91 (d, J=13.6 Hz, 1H), 3.73 (d, J=17.5 Hz, 1H), 3.56 (t, J=12.0 Hz, 2H), 3.46-3.34 (m, 2H), 3.20 (dd, J=28.5, 11.5 Hz, 3H), 2.90-2.83 (m, 1H), 2.80 (d, J=1.6 Hz, 3H), 2.65 (d, J=13.7 Hz, 1H), 2.29-2.20 (m, 2H), 2.02 (m, 5H), 1.87 (m, 1H). MS (ESI, m/e) [M+1]⁺537.4.

### Example 12: 3-(4-(3,8-diazabicyclo[3.2.1]octan-3-yl)-2-(((S)-1-methylpyrrolidin-2-yl)methoxy)-5,8-dihydropyrido[3,4-d]pyrimidin-7(6H)-yl)-5-chloro-4-(trifluoromethyl)aniline

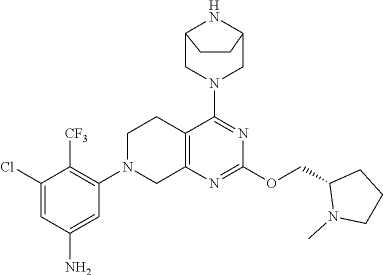 [SMILES: CN1CCC[C@H]1COc1nc2c(c(N3CC4CCC(C3)N4)n1)CCN(c1cc(N)cc(Cl)c1C(F)(F)F)C2]

### Step 1: tert-butyl (3-bromo-5-chloro-4-(trifluoromethyl)phenyl)(tert-butoxycarbonyl)carbamate

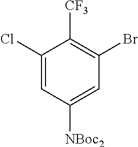

To a mixture of 3-bromo-5-chloro-4-(trifluoromethyl)aniline (273 mg, 1 mmol), DMAP(24.4 mg, 0.2 mmol) and TEA(0.5 mL) in THF (10 ml) was added di-tert-butyl dicarbonate (874 mg, 4 mmol) at rt. The resulting mixture was stirred for 16 hrs at 65° C. Upon completion, the reaction mixture was diluted with EtOAc (100 mL), washed with saturated NaCl (30 mL×3). The organic layer was dried over anhydrous Na₂SO₄, filtered and the filtrate was concentrated to give the residue. The residue was purified by Prep-TLC (EtOAc: PE=3:1) to give the title product (200 mg, 42%). MS (ESI, m/e) [M+1]⁺474.02.

### Step 2: tert-butyl 3-(7-(5-(bis(tert-butoxycarbonyl)amino)-3-chloro-2-(trifluoromethyl)phenyl)-2-(((S)-1-methylpyrrolidin-2-yl)methoxy)-5,6,7,8-tetrahydropyrido[3,4-d]pyrimidin-4-yl)-3,8-diazabicyclo[3.2.1]octane-8-carboxylate

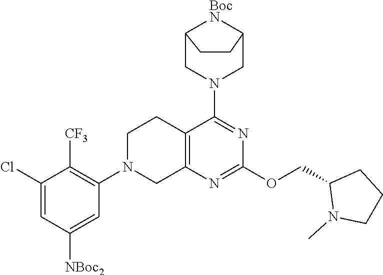

To a solution of tert-butyl 3-(2-(((S)-1-methylpyrrolidin-2-yl)methoxy)-5,6,7,8-tetrahydropyrido[3,4-d]pyrimidin-4-yl)-3,8-diazabicyclo[3.2.1]octane-8-carboxylate (46 mg, 0.1 mmol), tert-butyl (3-bromo-5-chloro-4-(trifluoromethyl)phenyl)(tert-butoxycarbonyl)carbamate (71 mg, 0.15 mmol), [1,3-bis[2,6-bis[3-methyl-1-(2-methylpropyl)butyl]phenyl]-4,5-dichloro-1,3-dihydro-2H-imidazol-2-ylidene]chloro[(1,2,3-η)-1-phenyl-2-propen-1-yl]-palladium (10.8 mg, 0.01 mmol), Pd₂(dba)₃ (9.2 mg, 0.01 mmol) and BINAP (12.4 mg, 0.02 mmol) in dioxane (20 mL) was added cesium carbonate (130.3 mg, 0.4 mmol) at room temperature. The resulting mixture was stirred at 100° C. for 16 h. After completed, the reaction mixture was quenched with water (20 mL), extracted with DCM (50 mL×3), the organic layers were dried over anhydrous Na₂SO₄, filtered and the filtrate was concentrated under reduced pressure to give the residue. The residue was purified by Prep-TLC (DCM: MeOH=10:1) to give the product (50 mg, 58.7%). MS (ESI, m/e) [M+1]⁺851.4.

### Step 3: 3-(4-(3,8-diazabicyclo[3 .2 .1]octan-3-yl)-2-4(5)-1-methylpyrrolidin-2-yl)methoxy)-5,8-dihydropyrido[3,4-d]pyrimidin-7(6H)-yl)-5-chloro-4-(trifluoromethyl)aniline

To a mixture of tert-butyl 3-(7-(5-(bis(tert-butoxycarbonyl)amino)-3-chloro-2-(trifluoromethyl)phenyl)-2-(((S)-1-methylpyrrolidin-2-yl)methoxy)-5,6,7,8-tetrahydropyrido[3,4-d]pyrimidin-4-yl)-3,8-diazabicyclo[3.2.1]octane-8-carboxylate (50 mg, 0.058 mmol) in DCM (5 mL) was added TFA (3 mL) at rt. The resulting mixture was stirred for 16 h at rt. Upon completion, the reaction mixture was concentrated under reduced pressure to give the residue. The residue was purified by prep-HPLC (ACN in water with 0.1% of FA, 0% to 90%) to give the title product (1.52 mg, 4.7%) . ¹H NMR (400 MHz, CD₃OD) δ 6.44 (s, 1H), 6.39 (s, 1H), 4.27-4.16 (m, 2H), 3.81-3.75 (s, 4H), 3.50-3.48 (m, 2H), 3.21-3.14 (m, 5H), 2.70-2.65 (m, 3H), 2.42 (s, 3H), 2.31-2.25 (m, 1H), 2.05-1.93 (m, 1H), 1.77-1.67 (m, 6H), 1.61-1.57 (m, 1H). MS (ESI, m/e) [M+1]⁺552.25.

### Example 13: 3-(4-(3,8-diazabicyclo[3.2.1]octan-3-yl)-2-0(S)-1-methylpyrrolidin-2-yl)methoxy)-5,8-dihydropyrido[3,4-d]pyrimidin-7(6H)-yl)-4,5-dichloroaniline

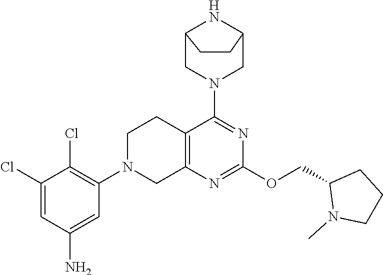 [SMILES: CN1CCC[C@H]1COc1nc2c(c(N3CC4CCC(C3)N4)n1)CCN(c1cc(N)cc(Cl)c1Cl)C2]

Example 13 was prepared by similar procedure as described in Example 12 from 3-bromo-4,5-dichloroaniline. ¹H NMR (400 MHz, CD₃OD) δ 6.58 (d, J=2.4 Hz, 1H), 6.46 (d, J=2.4 Hz, 1H), 4.57 (dd, J=12.1, 3.8 Hz, 1H), 4.49-4.45 (m, 1H), 4.15 (d, J=13.8 Hz, 2H), 4.1-4.05 (m, 4H), 3.58-3.48 (m, 2H), 3.39 (d, J=13.8 Hz, 3H), 3.25 (t, J=5.2 Hz, 2H), 3.04-2.94 (m, 1H), 2.94-2.84 (m, 4H), 2.33-2.26 (m, 1H), 2.18-1.87 (m, 7H). MS (ESI, m/e) [M+1]⁺518.4.

### Example 14: 4-(3,8-diazabicyclo[3.2.1]octan-3-yl)-7-(8-chloronaphthalen-1-yl)-2-(((2S,4R)-4-methoxy-1-methylpyrrolidin-2-yl)methoxy)-5,6,7,8-tetrahydropyrido[3,4-d]pyrimidine

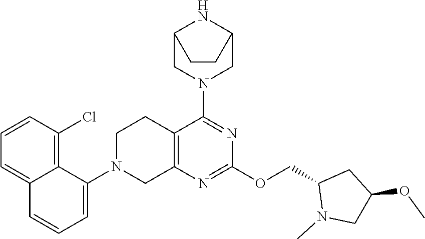 [SMILES: CO[C@@H]1C[C@@H](COc2nc3c(c(N4CC5CCC(C4)N5)n2)CCN(c2cccc4cccc(Cl)c24)C3)N(C)C1]

Example 14 was prepared by similar procedure as described in Example 7 from ((2S,4R)-4-methoxy-1-methylpyrrolidin-2-yl)methanol. ¹H NMR (400 MHz, CD₃OD) δ 7.89-7.75 (m, 1H), 7.70 (d, J=7.8 Hz, 1H), 7.55-7.41 (m, 2H), 7.45-7.27 (m, 2H), 4.87-4.83 (m, 3H), 4.73-4.52 (m, 1H), 4.45-4.33 (m, 1H), 4.21-1.41 (m, 3H), 4.12-3.96 (m, 2H), 3.89-3.73 (m, 2H), 3.63-3.52 (m, 2H), 3.46-3.31 (m, 4H), 3.22-3.10 (m, 4H), 2.73-2.59 (m, 1H), 2.49-2.33 (m, 2H), 2.24-2.01 (m, 4H). MS (ESI, m/e) [M+1]⁺549.4.

### Example 15: 4-(3,8-diazabicyclo[3.2.1]octan-3-yl)-7-(8-chloronaphthalen-1-yl)-2-(((2S,4R)-4-fluoro-1-methylpyrrolidin-2-yl)methoxy)-5,6,7,8-tetrahydropyrido[3,4-d]pyrimidine

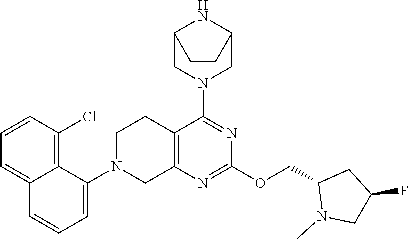 [SMILES: CN1C[C@H](F)C[C@H]1COc1nc2c(c(N3CC4CCC(C3)N4)n1)CCN(c1cccc3cccc(Cl)c13)C2]

Example 15 was prepared by similar procedure as described in Example 7 from ((2S,4R)-4-fluoro-1-methylpyrrolidin-2-yl)methanol. ¹H NMR (400 MHz, CD₃OD) δ 7.83 (d, J=8.1 Hz, 1H), 7.68 (d, J=8.1 Hz, 1H), 7.54-7.43 (m, 2H), 7.44-7.28 (m, 2H), 5.29-5.15 (m, 1H), 4.49-4.26 (m, 4H), 4.14-4.10 (m, 3H), 3.92 (d, J=13.4Hz, 1H), 3.73 (d, J=17.6 Hz, 1H), 3.61-3.55(m, 3H), 3.30-3.11 (m, 3H), 2.83 (dd, J=29.0, 12.2 Hz, 1H), 2.66-2.64 (m, 4H), 2.37-2.29 (m, 2H), 2.17-2.01 (m, 4H). MS (ESI, m/e) [M+1]⁺537.4.

### Example 16: (2S,4R)-14(S)-2-(74(S)-2-(((4-(3,8-diazabicyclo[3.2.1]octan-3-yl)-7-(8-chloronaphthalen-1-yl)-5,6,7,8-tetrahydropyrido[3,4-d]pyrimidin-2-yl)oxy)methyl)pyrrolidin-1-yl)heptanamido)-3,3-dimethylbutanoyl)-4-hydroxy-N-((S)-1-(4-(4-methylthiazol-5-yl)phenyl)ethyl)pyrrolidine-2-carboxamide

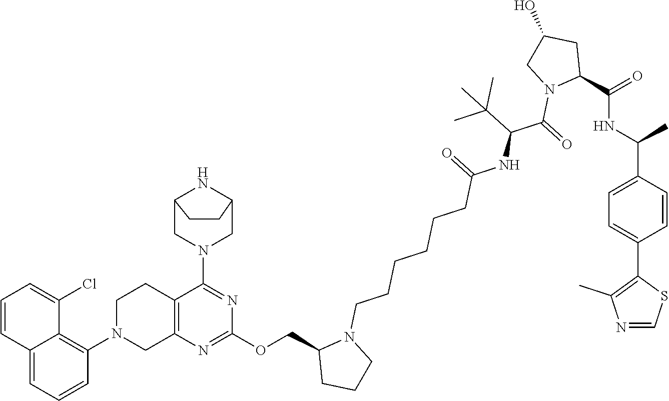 [SMILES: Cc1ncsc1-c1ccc([C@H](C)NC(=O)[C@@H]2C[C@@H](O)CN2C(=O)[C@@H](NC(=O)CCCCCCN2CCC[C@H]2COc2nc3c(c(N4CC5CCC(C4)N5)n2)CCN(c2cccc4cccc(Cl)c24)C3)C(C)(C)C)cc1]

Example 16 was prepared by similar procedure as described in Example 17 from methyl (S)-7-(2-(hydroxymethyl)pyrrolidin-1-yl)heptanoate. ¹H NMR (500 MHz, DMSO-d6) δ 8.98 (s, 1H), 8.36 (d, J=10 Hz, 1H), 7.92 (d, J=10 Hz, 1H), 7.77-7.73 (m, 2H), 7.57-7.52 (m, 2H), 7.42-7.38 (m, 3H), 7.37-7.33 (m, 3H), 5.11-5.08 (s, 1H), 4.93-4.90 (m, 1H), 4.50 (d, J=10 Hz, 1H), 4.41 (t, J=10 Hz, 1H), 4.28-4.21 (m, 2H), 4.18-4.08 (m, 2H), 3.98-3.93 (m, 3H), 3.76-3.70 (m, 2H), 3.60-3.59 (m, 2H), 3.50-3.48 (m, 1H), 3.13-3.06 (m, 5H), 2.81-2.77 (m, 2H), 2.44 (s, 3H), 2.32-2.29 (m, 1H), 2.23-2.18 (m, 2H), 2.08-1.98 (m, 3H), 1.89-1.76 (m, 5H), 1.68-1.59 (m, 3H), 1.47-1.34 (m, 6H), 1.23-1.21 (s, 4H), 0.91 (s, 9H). MS (ESI, m/e) [M+1]⁺1059.80.

### Example 17: (2S,4R)-1-((S)-2-(3-(3-((S)-2-(((4-(3,8-diazabicyclo[3.2.1]octan-3-yl)-7-(8-chloronaphthalen-1-yl)-5,6,7,8-tetrahydropyrido[3,4-d]pyrimidin-2-yl)oxy)methyppyrrolidin-1-yl)propoxy)propanamido)-3,3-dimethylbutanoyl)-4-hydroxy-N-((S)-1-(4-(4-methylthiazol-5-yl)phenyl)ethyl)pyrrolidine-2-carboxamide

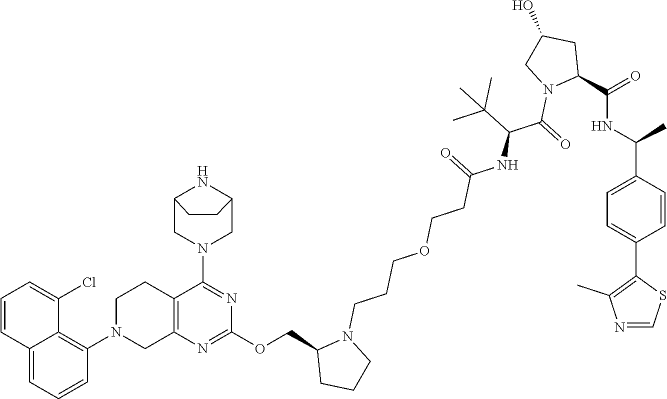 [SMILES: Cc1ncsc1-c1ccc([C@H](C)NC(=O)[C@@H]2C[C@@H](O)CN2C(=O)[C@@H](NC(=O)CCOCCCN2CCC[C@H]2COc2nc3c(c(N4CC5CCC(C4)N5)n2)CCN(c2cccc4cccc(Cl)c24)C3)C(C)(C)C)cc1]

### Step 1: benzyl 4-(8-(tert-butoxycarbonyl)-3,8-diazabicyclo[3.2.1]octan-3-yl)-2-(((S)-1-(3-(3-methoxy-3-oxopropoxy)propyl)pyrrolidin-2-yl)methoxy)-5,8-dihydropyrido[3,4-d]pyrimidine -7(6H)-carboxylate

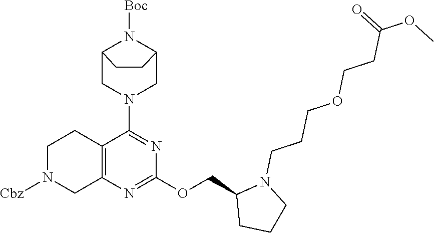 [SMILES: COC(=O)CCOCCCN1CCC[C@H]1COc1nc2c(c(N3CC4CCC(C3)N4C(=O)OC(C)(C)C)n1)CCN(C(=O)OCc1ccccc1)C2]

To a mixture of benzyl 4-(8-(tert-butoxycarbonyl)-3,8-diazabicyclo[3.2.1]octan-3-yl)-2-chloro-5,8-dihydropyrido[3,4-d]pyrimidine-7(6H)-carboxylate (513 mg, 1.0 mmol), methyl (S)-3-(3-(2-(hydroxymethyppyrrolidin-1-yl)propoxy)propanoate (368 mg, 1.5 mmol), Cs₂CO₃ (978 mg, 3.0 mmol), Pd₂(dba)₃ (183 mg, 0.2 mmol) and RuPhos (183 mg, 0.4 mmol) was added toluene (20 mL). The mixture was stirred at 100° C. for 15 hrs. The resulting cooled mixture was concentrated and purified by column chromatography (DCM/MeOH=10/1) to give the title product (356 mg, 49%). MS (ESI, m/e) [M+1]⁺723.5

### Step 2: tert-butyl 3-(2-(((S)-1-(3-(3-methoxy-3-oxopropoxy)propyl)pyrrolidin-2-yl)methoxy)-5,6,7,8-tetrahydropyrido[3,4-d]pyrimidin-4-yl)-3,8-diazabicyclo[3.2.1]octane-8-carboxylate

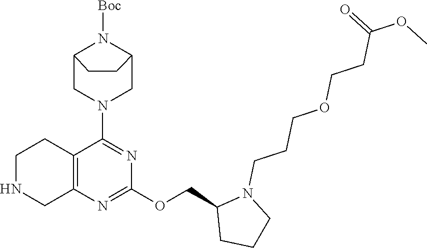 [SMILES: COC(=O)CCOCCCN1CCC[C@H]1COc1nc2c(c(N3CC4CCC(C3)N4C(=O)OC(C)(C)C)n1)CCNC2]

To a solution of benzyl 4-(8-(tert-butoxycarbonyl)-3,8-diazabicyclo[3.2.1]octan-3-yl)-2-(((S)-1-(3-(3-methoxy-3-oxopropoxy)propyl)pyrrolidin-2-yl)methoxy)-5,8-dihydropyrido[3,4-d]pyrimidine-7(6H)-carboxylate (356 mg, 0.49 mmol) in methanol (25 mL) was added 10% wet Pd/C (170 mg). The mixture was stirred at room temperature for 2 hrs under hydrogen atmosphere. Then it was filtered and the filtrate was evaporated to give the title product (240 mg, 83%). MS (ESI, m/e) [M+1]⁺589.4.

### Step 3: tert-butyl 3-(7-(8-chloronaphthalen-1-yl)-2-(((S)-1-(3-(3-methoxy-3-oxopropoxy)propyl)pyrrolidin-2-yl)methoxy)-5,6,7,8-tetrahydropyrido[3,4-d]pyrimidin-4-yl)-3,8-diazabicyclo[3.2.1]octane-8-carboxylate

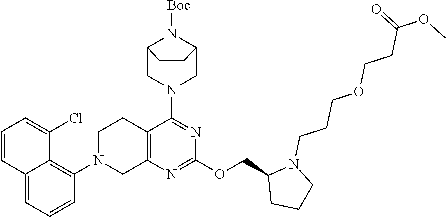 [SMILES: COC(=O)CCOCCCN1CCC[C@H]1COc1nc2c(c(N3CC4CCC(C3)N4C(=O)OC(C)(C)C)n1)CCN(c1cccc3cccc(Cl)c13)C2]

To a mixture of tert-butyl 3-(2-(((S)-1-(3-(3-methoxy-3-oxopropoxy)propyl)pyrrolidin-2-yl)methoxy)-5,6,7,8-tetrahydropyrido[3,4-d]pyrimidin-4-yl)-3,8-diazabicyclo[3.2.1]octane-8-carboxylate (120 mg, 0.20 mmol), 1-bromo-8-chloronaphthalene (245 mg, 1.02 mmol), Cs₂CO₃ (200 mg, 0.61 mmol), Pd₂(dba)₃ (56 mg, 0.06 mmol) and RuPhos (56 mg, 0.12 mmol) was added toluene (10 mL). The mixture was stirred at 100° C. for 4 hrs. The resulting cooled mixture was concentrated and purified by column chromatography (DCM/MeOH=10/1) to give the title product (60 mg, 40%). MS (ESI, m/e) [M+1]⁺749.5.

### Step 4: 3-(3-(S)-2-(((4-(8-(tert-butoxycarbonyl)-3,8-diazabicyclo[3.2.1]octan-3-yl)-7-(8-chloronaphthalen-1-yl)-5,6,7,8-tetrahydropyrido[3,4-d]pyrimidin-2-yl)oxy)methyl)pyrrolidin-1-yl)propoxy)propanoic acid

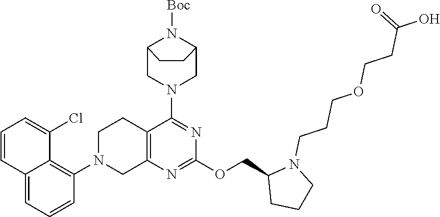 [SMILES: CC(C)(C)OC(=O)N1C2CCC1CN(c1nc(OC[C@@H]3CCCN3CCCOCCC(=O)O)nc3c1CCN(c1cccc4cccc(Cl)c14)C3)C2]

To a solution of tert-butyl 3-(7-(8-chloronaphthalen-1-yl)-2-(((S)-1-(3-(3-methoxy-3-oxopropoxy)propyl)pyrrolidin-2-yl)methoxy)-5,6,7,8-tetrahydropyrido[3,4-d]pyrimidin-4-yl)-3,8-diazabicyclo[3.2.1]octane-8-carboxylate (60 mg, 0.08 mmol) in methanol (2.5 mL) was added THF (2.5 mL) and LiOH/H₂O (1M, 2 mL). The mixture was stirred at room temperature for 0.5 h. Then, it was neutralized by HCl/H₂O (1M) to pH=5-6. Solvent was evaporated and the residue was dissolved in DCM (10 mL) and filtered. The filtrate was concentrated and dried to give the title product (58 mg, 99%). MS (ESI, m/e) [M+1]⁺735.5.

### Step 5: tert-butyl 3-(7-(8-chloronaphthalen-1-yl)-2-(((S)-1-(3-(3-(((S)-1-((2S,4R)-4-hydroxy-2-(((S)-1-(4-(4-methylthiazol-5-yl)phenyl)ethyl)carbamoyl)pyrrolidin-1-yl)-3,3-dimethyl-1-oxobutan-2-yl)amino)-3-oxopropoxy)propyl)pyrrolidin-2-yl)methoxy)-5,6,7,8-tetrahydropyrido[3,4-d]pyrimidin-4-yl)-3,8-diazabicyclo[3.2.1]octane-8-carboxylate

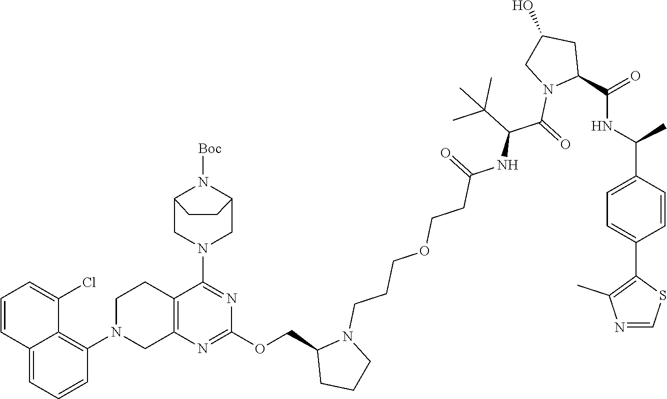 [SMILES: Cc1ncsc1-c1ccc([C@H](C)NC(=O)[C@@H]2C[C@@H](O)CN2C(=O)[C@@H](NC(=O)CCOCCCN2CCC[C@H]2COc2nc3c(c(N4CC5CCC(C4)N5C(=O)OC(C)(C)C)n2)CCN(c2cccc4cccc(Cl)c24)C3)C(C)(C)C)cc1]

To a mixture of 3-(34(S)-2-(44-(8-(tert-butoxycarbonyl)-3,8-diazabicyclo[3.2.1]octan-3-yl)-7-(8-chloronaphthalen-1-yl)-5,6,7,8-tetrahydropyrido[3,4-d]pyrimidin-2-yl)oxy)methyppyrrolidin-1-y1)propoxy)propanoic acid (58 mg, 0.08 mmol), (2S,4R)-4-((S)-2-amino-3,3-dimethylbutanoyl)-4-hydroxy-N-((S)-1-(4-(4-methylthiazol-5-yl)phenyl)ethyl)pyrrolidine-2-carboxamide hydrochloride (38 mg, 0.08 mmol) and HATU (45 mg, 0.12 mmol) was added DCM (5 mL) and DMF (5 mL), then DIPEA (31 mg, 0.24 mmol) was added and the mixture was stirred at room temperature for 1 h. The resulting solution was washed with brine (10 mL), water (10 mL) and dried over Na₂SO₄. Solvent was concentrated and the residue was purified by Prep-TLC (DCM/MeOH=10/1) to give the title product (90 mg, 98%). MS (ESI, m/e) [M+2H]2+ 581.6.

### Step 6: (2S,4R)-1-((S)-2-(3-(((4-(S)-2-(((4-(3,8-diazabicyclo[3.2.1]octan-3-yl)-7-(8-chloronaphthalen-1-yl)-5,6,7,8-tetrahydropyrido[3,4-d]pyrimidin-2-yl)oxy)methyl)pyrrolidin-1-yl)propoxy)propanamido)-3,3-dimethylbutanoyl)-4-hydroxy-N-((S)-1-(4-(4-methylthiazol-5-yl)phenyl)ethyl)pyrrolidine-2-carboxamide

To a solution of tert-butyl 3-(7-(8-chloronaphthalen-1-yl)-2-(((S)-1-(3-(3-(((S)-1-((2S,4R)-4-hydroxy-2-(((S)-1-(4-(4-methylthiazol-5-yl)phenyl)ethyl)carbamoyl)pyrrolidin-1-yl)-3,3-dimethyl-1-oxobutan-2-yl)amino)-3-oxopropoxy)propyl)pyrrolidin-2-yl)methoxy)-5,6,7,8-tetrahydropyrido[3,4-d]pyrimidin-4-yl)-3,8-diazabicyclo[3.2.1]octane-8-carboxylate (90 mg, 0.08 mmol) in DCM (15 mL) was added TFA (3 mL). The mixture was stirred at room temperature for 1 hour. The resulting solution was concentrated and purified by Prep-HPLC (ACN in water with 0.1% of FA, 0% to 90%) to give the title product (25 mg, 30%). ¹H NMR (400 MHz, CD₃OD) δ 8.87 (s, 1H), 7.82 (d, J=8.2 Hz, 1H), 7.68 (d, J=8.1 Hz, 1H), 7.53-7.48 (m, 2H), 7.42-7.32 (m, 6H), 4.99-4.96 (m, 1H), 4.66-4.50 (m, 4H), 4.40-4.32 (m, 3H), 4.12 (s, 2H), 3.95-3.87 (m, 2H), 3.82-3.81 (m, 1H), 3.73-3.70 (m, 3H), 3.65-3.57 (m, 6H), 3.27-3.18 (m, 6H), 2.68-2.65 (m, 4H), 2.51-2.43 (m, 5H), 2.35-2.28 (m, 2H), 2.20-2.16 (m, 1H), 2.14-2.08 (m, 2H), 2.05-1.98 (m, 3H), 1.96-1.92 (m, 1H), 1.56-1.48 (m, 3H), 1.00 (s, 9H). MS (ESI, m/e) [M+H]+ 1061.8.

### METHODS

### KRAS: SOS1 GDP TR-FRET Assay

This assay was used to identify compounds which competitively interact with the binding of KRAS protein to SOS1 in the presence of GDP. GST-tagged WT KRAS (amino acids 1-188), GST-tagged KRAS (amino acids 1-188) G12D and His- tagged SOS1 protein (amino acids 564-1049) was expressed in E. coli and purified. All protein and reaction solutions were prepared in assay buffer containing DPBS pH7.5, 0.1% BSA, and 0.05% Tween 20. Purified GST-tagged WT KRAS or KRAS G12D protein (37.5 nM final assay concentration) and GDP (Sigma, 10 μM final assay concentration) mixture, a serially diluted compound (top final concentration is 50 uM or 10 uM, 3-fold serially diluted, 10 points) and His-tagged SOS1 protein (18 nM final assay concentration) were added into the assay plates (384 well microplate, black, Corning) sequentially. Plates are incubated at 24° C. for 1 hr. Following the incubation, Mab Anti-6His-Tb cryptate (Cisbio) and Mab Anti GST-D2 (Cisbio) were added and further incubated at 24° C. for another 1 hr. The TR-FRET signals (ex337nm, em665nm/620nm) were read on BMG PHERAstar FSX instrument. The inhibition percentage of KRAS protein binding with SOS1 in presence of increasing concentrations of compounds was calculated based on the ratio of fluorescence at 665 nm to that at 620 nm. The IC₅₀ value of each compound was calculated from fitting the data to the four-parameter logistic model by Dotmatics.

### pERK Assay

AsPC-1 cell line was used in this study. Cells were maintained in RPMI-1640 supplemented with 10% fetal bovine serum (Thermo Fisher), 50 units/mL penicillin and streptomycin (Thermo Fisher) and kept at 37° C. in a humidified atmosphere of 5% CO₂ in air. Cells were reinstated from frozen stocks that were laid down within 30 passages from the original cells purchased. 30000 cells per well were seeded into a 96-well plate and incubated overnight. Cells were treated with a 10-point dilution series. The final compound concentration is from 0 to 10 μM. After 2 h compound treatment, cells were lysed, and the pERK1/2(THR_{202/}TYR₂₀₄) level in the cell lysates was detected by HTRF kit (Cisbio). In brief, a total of 16 μl of cell lysate from each well of a 96-well plate was transferred to a 384-well white assay plate. Lysate from each well was incubated with 2 μL of Eu3+- cryptate (donor) labeled anti-phospho-ERK1/2 and 2 μL of D2 (acceptor) labeled anti-phospho-ERK1/2 antibodies (Cisbio) overnight in dark at room temperature. When donor and acceptor are in close proximity, excitation of the donor with laser triggers a Fluorescence Resonance Energy Transfer (FRET) towards the acceptor, which in turn fluoresces at 655 nm wavelength. FRET signals were measured using a PHERAstar FSX reader (BMG Labtech). IC₅₀ determination was performed by fitting the curve of percent inhibition versus the log of the inhibitor concentration using Dotmatics.

### Data Analysis

| | KRAS^{G12D}: | KRAS^{WT}: | |
|---|---|---|---|
| | SOS1 GDP TR-FRET | SOS1 GDP TR-FRET | pERK |
| Example No. | assay IC₅₀ (nM) | assay IC₅₀ (nM) | IC₅₀ (nM) |
| 1 | 22706 | >50000 | |
| 2 | 4499 | 34180 | |
| 3 | 635 | >50000 | |
| 4 | 2589 | >50000 | |
| 5 | 118 | >50000 | >10000 |
| 6 | 713 | >50000 | |
| 7 | 59 | 17232 | 6755 |
| 8 | 749 | >10000 | |
| 9 | 63 | >10000 | 9927 |
| 10 | 22 | 6208 | |
| 11 | 12 | 3624 | 4639 |
| 12 | 9.8 | 2393 | 1948 |
| 13 | 27 | >10000 | 4097 |
| 14 | 126 | >10000 | >10000 |
| 15 | 41 | >10000 | 4029 |

### KRAS G12D NanoLuc Assay

HEK293 KRAS-G12D NanoLuc cell pool was used in this study. The cells were stable expressing KRAS G12D HiBiT and LgBiT. Cells were maintained in DMEM supplemented with 10% fetal bovine serum (Thermo Fisher), 50 units/mL penicillin and streptomycin (Thermo Fisher) and kept at 37° C. in a humidified atmosphere of 5% CO₂ in air. Cells were reinstated from frozen stocks that were laid down within 30 passages from the cell pool was constructed. 20000 cells per well were seeded a 96-well White with Clear Flat bottom plate for 4 h. Cells were treated with a 10-point dilution series. The final compound concentration is from 0 to 10 μM. After 24 h compound treatment, added 20 μL of CellTiter-Fluor reagent (Promega) to plates, shook for 2 min, incubated at least 30 min in 37° C. Added 25 μL of NanoGlo Live cell reagent (Promega) to the same plate. shook by mini-shaker for 5 min. Measure Luminescence and Fluorescence (380-400nmEx/505nmEm) signals immediately using a PHERAstar FSX reader (BMG Labtech). DC₅₀ determination was performed by fitting the curve of percent inhibition versus the log of the inhibitor concentration using Excel and GraphPad Prism 8.

### Data Analysis

| | KRAS^{G12D} | KRAS^{G12D} |
|---|---|---|
| Example No. | NanoLuc DC50 (nM) | NanoLuc Dmax |
| 16 | 1004 | 73% |
| 17 | 708 | 80% |

### Western Blotting Assay

AsPC-1 cell line was used in this study. Cells were maintained in RPMI-1640 supplemented 10% fetal bovine serum (Thermo Fisher), 50 units/mL penicillin and streptomycin (Thermo Fisher) and kept at 37° C. In a humidified atmosphere of 5% CO₂ in air. Cells were reinstated from frozen stocks that were laid down within 30 passages from the original cells purchased. 400000 AsPC-1 cells per well in 1 mL culture medium were seed in the 12-well plate for 4 hours. Cells were treated with an appropriate dilution series of compounds. After 48h compound treatment, medium was aspirated, the cells were washed with PBS, and then 30 μL of 1× protein lysis buffer (Cell Signaling Technology) containing protease inhibitors (Merck) and phosphatase inhibitors (Sigma) was added. The cells were lysed, and after centrifugation, the supernatants were quantified by BCA Protein assay Kit (Thermo Fisher). 4× loading Buffer (Thermo Fisher) was added to equal amounts of total protein from each sample and heated at 95° C. for 5 minutes. 30-50 μg of cell lysates were loaded onto a 12% NuPAGE Bis-Tris Gel (Thermo Fisher), electro-transferred to NC membranes (Thermo Fisher), The membranes were blocked at least 1 hour with Blocking reagent (LI-COR), and then incubated overnight with anti-KRAS (LSBio, LS-C₁₇₅₆₆₅) or p-ERK (Cell Signaling Technology, 4370L) antibodies and as loading control anti-β-actin (Cell Signaling Technology, 3700S) or anti-GAPDH (Cell Signaling Technology, 97166S) at 4° C. with gentle shaking. The membranes were washed three times with TBST, and incubated for at least 1 hour at room temperature with anti-mouse or anti-Rabbit secondary fluorescent antibody (Thermo Fisher, A32729; LI-COR, 926-32213). The membranes were washed three times in TBST, and one time in water. Immunoreactive bands were visualized by Odyssey CLx.

### Data Analysis

| Example No. | KRAS^{G12D} protein level decrease | Concentration (uM) |
|---|---|---|
| 17 | 40% | 3 |

It is to be understood that, if any prior art publication is referred to herein; such reference does not constitute an admission that the publication forms a part of the common general knowledge in the art in any country.

In the claims which follow and in the preceding description of the invention, except where the context requires otherwise due to express language or necessary implication, the word “comprise” or variations such as “comprises” or “comprising” is used in an inclusive sense, i.e., to specify the presence of the stated features but not to preclude the presence or addition of further features in various embodiments of the invention.

The disclosures of all publications, patents, patent applications and published patent applications referred to herein by an identifying citation are hereby incorporated herein by reference in their entirety.

Although the foregoing invention has been described in some detail by way of illustration and example for purposes of clarity of understanding, it is apparent to those skilled in the art that certain minor changes and modifications will be practiced. Therefore, the description and examples should not be construed as limiting the scope of the invention.

1. A bridged compound of Formula (I): 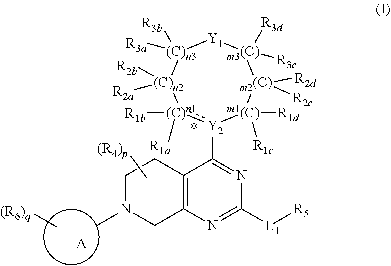
or a pharmaceutically acceptable salt thereof, or a stereoisomer thereof,
wherein:
Ring A is an aryl group or a 5- to 7-membered monocyclic heteroaryl or 8- to 12-membered bicyclic heteroaryl group; [symbol]
is each independently a single bond or double bond;
Y₁ is —NH— or —C(R_{Y1a})(NHR_{Y1b})—;
Y₂ is N or CR_{Y2} in the case that [symbol] is a single bond; or Y₂ is C and R_{1b} is absent in the case that [symbol] is a double bond;
n1, n2, n3, m1, m2, and m3 are each independently 0 or 1, provided that at least one of n1, n2 and n3 is 1; and at least one of m1, m2 and m3 is 1;
p is 0, 1, 2, 3, 4, 5 or 6;
q is 0, 1, 2, 3, 4, 5, 6 or 7 provided that the valence theory is met;
R₁ₐ, R_{1b}, R₂, R_{2b}, R₃ₐ, R_{3b}, R_{1c}, R_{1d}, R_{2c}, R_{2d}, R_{3c}, R_{3d}, R_{Y1}a, R_{Y1b} and R_{Y2}, if present, are each independently hydrogen, halogen, —C₁₋₈alkyl, C₃-C₈cycloalkyl, 3- to 8-membered heterocyclyl, C₆-C₁₂aryl, 5- to 12-membered heteroaryl, —CN, —OR_{1c}, or —NR₁ₑR_{1f}; wherein each of said —C₁₋₈alkyl, C₃-C₈cycloalkyl, 3- to 8-membered heterocyclyl, C₆-C₁₂aryl or 5- to 12-membered heteroaryl is optionally substituted with at least one substituent R_{1g};
at least one pair of (R₁ₐ and R_{1c}), (R₁ₐ and R_{2c}), (R₁ₐ and R_{3c}), (R₂ₐ and R_{1c}), (R₂ₐ and R_{2c}), (R₂ₐ and R_{3c}), (R₃ₐ and R_{1c}), (R₃ₐ and R_{2c}), (R₃ₐ and R_{3c}), (R_{Y1a} and R_{Y2}), (R_{Y1a} and R₁ₐ), (R_{Y1a} and R₂ₐ), (R_{Y1a} and R_{1c}), (R_{Y1a} and R_{2c}), (R_{Y1b} and R₁ₐ), (R_{Y1b} and R₂ₐ), (R_{Y1b} and R_{1c}), (R_{Y1b} and R_{2c}), and (R_{Y1b} and R_{Y2}) form a bridge containing one, two, three, or four —CH₂— moieties in addition to the two bridgeheads, wherein one of the —CH₂— moiety is optionally replaced with —O—, —S—or —NH— and wherein said bridge is optionally substituted with at least one substituent R_{1g};
optionally, (R_{Y1a} and R_{Y1b}), (R_{Y1a} and R₃ₐ ), (R_{Y1a} and R_{3c}), (R_{Y1b} and R₃ₐ ), or (R_{Y1b} and R_{3c}) form 3- to 12-membered ring, the said ring comprises 0-3 heteroatoms selected from nitrogen, sulfur and oxygen and the said bridge is optionally substituted with at least one substituent R_{1g};
R₁ₑ and R_{1f} are each independently selected from hydrogen, —C₁₋₈alkyl, —C₂₋₈alkenyl, —C₂₋₈alkynyl, C₁₋₈alkoxy-C₁₋₈alkyl-, C₃-C₈cycloalkyl, 3- to 8-membered heterocyclyl, C₆-C₁₂aryl or 5- to 12-membered heteroaryl;
R_{1g}, at each occurrence, is independently halogen, —C₁₋₈alkyl, —C₂₋₈alkenyl, —C₂₋₈alkynyl, —C₁₋₈cycloalkyl, 3- to 8-membered heterocyclyl, —C₆-C₁₂aryl, or 5- to 12-membered heteroaryl, —C₁₋₈haloalkyl, C₁₋₈alkoxy-C₁₋₈alkyl-, —CN, —OH, —NH₂, —C₁₋₈alkoxyl, —COOH, -or CO—C₁₋₈alkyl;
R₆ is hydrogen, halogen, —C₁₋₈alkyl, —C₂₋₈alkenyl, —C₂₋₈alkynyl, C₃-C₈cycloalkyl, 3- to 8-membered heterocyclyl, C₆-C₁₂aryl, 5- to 12-membered heteroaryl, —CN, oxo, —OR₆ₐ, —SR₆ₐ, —SO₂R₆ₐ, —SO₂NR₆ₐ R_{6b}, —COR₆ₐ, —CO₂R₆ₐ, —CONR₆ₐR_{6b}, —NR₆ₐR_{6b}, —NR₆ₐCOR_{6b}, —NR₆ₐCO₂R_{6b}, or —NR₆ₐSO₂R_{6b}; each of said —C₁₋₈alkyl, C₃-C₈cycloalkyl, 3- to 8-membered heterocyclyl, C₆-C₁₂aryl or 5- to 12-membered heteroaryl is optionally substituted with at least one substituent R_{6c};
R₆ₐ and R_{6b}are each independently selected from hydrogen, —C₁₋₈alkyl, —C₂₋₈alkenyl, —C₂₋₈alkynyl, C₁₋₈alkoxy-C₁₋₈alkyl-, C₃-C₈cycloalkyl, 3- to 8-membered heterocyclyl, C₆-C₁₂aryl or 5- to 12-membered heteroaryl, each of said —C₁₋₈alkyl, C₃-C₈cycloalkyl, 3- to 8-membered heterocyclyl, C₆-C₁₂aryl or 5- to 12-membered heteroaryl is optionally substituted with at least one substituent R_{6d};
R_{6c}, at each occurrence, is independently halogen, —C₁₋₈alkyl, —C₂₋₈alkenyl, —C₂₋₈alkynyl, —C₃-C₈cycloalkyl, 3- to 8-membered heterocyclyl, C₆-C₁₂aryl, or 5- to 12-membered heteroaryl; or two R₆ together with the atoms to which they are attached, form a 5, 6, 7, or 8-membered unsaturated or saturated ring, said ring comprising 0-3 heteroatoms independently selected from nitrogen, oxygen or sulfur; said ring is optionally substituted with at least one substituent R_{6d};
R_{6d} is hydrogen, halogen, —C₁₋₈alkyl, —C₂₋₈alkenyl, —C₂₋₈alkynyl, C₃-C₈cycloalkyl, 3- to 8-membered heterocyclyl, C₆-C₁₂aryl, 5- to 12-membered heteroaryl, —CN, oxo, —OR₆ₑ, —SO₂R₆ₑ, —SO₂NR₆ₑR_{6f} , —COR₆ₑ, —CO₂R₆ₑ, —CONR₆ₑR_{6f}, —NR₆ₑR_{6f}, —NR₆ₑCOR_{6f}, —NR₆ₑCO₂R_{6f}, or —NR₆ₑSO₂R_{6f} ; each of said C₁₋₈alkyl, —C₂₋₈alkenyl, —C₂₋₈alkynyl, C₃-C₈cycloalkyl, 3- to 8-membered heterocyclyl, C₆-C₁₂aryl or 5- to 12-membered heteroaryl is optionally substituted with at least one substituent R_{6g};
R₆ₑ and R_{6f} are each independently selected from hydrogen, —C₁₋₈alkyl, —C₂₋₈alkenyl, —C₂₋₈alkynyl, C₁₋₈alkoxy-C₁₋₈alkyl-, C₃-C₈cycloalkyl, 3- to 8-membered heterocyclyl, C₆-C₁₂aryl or 5- to 12-membered heteroaryl;
R_{6g}, at each occurrence, is independently halogen, —C₁₋₈alkyl, —C₂₋₈alkenyl, —C₂₋₈alkynyl, C₃-C₈cycloalkyl, 3- to 8-membered heterocyclyl, —C₆-C₁₂aryl, or 5- to 12-membered heteroaryl;
R₄ is hydrogen, halogen, —C₁₋₈alkyl, C₃-C₈cycloalkyl, —C₂₋₈alkenyl, —C₂₋₈alkynyl, 3- to 8-membered heterocyclyl, C₆-C₁₂aryl, 5- to 12-membered heteroaryl, —CN, oxo, —OR₄ₐ, —SR₄ₐ, —SO₂R₄ₐ, —SO₂NR₄ₐR_{4b}, —COR₄ₐ, —CO₂R₄ₐ, —CONR₄ₐR_{4b}, —NR₄ₐR_{4b}, —NR₄ₐCOR_{4b}, —NR₄ₐCO₂R_{4b}, or —NR₄ₐSO₂R_{4b}; each of said —C₁₋₈alkyl, C₃-C₈cycloalkyl, —C₂₋₈alkenyl, —C₂₋₈alkynyl, 3- to 8-membered heterocyclyl, C₆-C₁₂aryl or 5- to 12-membered heteroaryl is optionally substituted with at least one substituent R_{4c}, or two R₄ join each other to form a spiro cycle or bicycle
R₄ₐ and R_{4b} are each independently selected from hydrogen, —C₁₋₈alkyl, —C₂₋₈alkenyl, —C₂₋₈alkynyl, C₁₋₈alkoxy-C₁₋₈alkyl-, C₃-C₈cycloalkyl, 3- to 8-membered heterocyclyl, C₆-C₁₂aryl or 5- to 12-membered heteroaryl; each of said —C₁₋₈alkyl, —C₂₋₈alkenyl, —C₂₋₈alkynyl, C₁₋₈alkoxy-C₁₋₈alkyl-, C₃-C₈cycloalkyl, 3- to 8-membered heterocyclyl, C₆-C₁₂aryl or 5- to 12-membered heteroaryl is optionally substituted with at least one substituent R_{4d};
R_{4c} and R_{4d}, at each occurrence, are each independently halogen, —C₁₋₈alkyl, —C₂₋₈alkenyl, —C₂₋₈alkynyl, —C₃-C₈cycloalkyl, 3- to 8-membered heterocyclyl, —C₆-C₁₂aryl, or 5- to 12-membered heteroaryl;
L₁ is selected from a single bond, —O—, —NR^{L1a}—, —C(O)—, —C₁₋₈alkylene-, *L₁-O—C₁₋₈alkylene-**L₁, —C₃-C₈cycloalkylene, *^{L1}-O—C₃-C₈cycloalkylene-**^{L1}, *^{L1}-O—C₁₋₈alkylene-NR^{L1a}-**^{L1}, *^{L1}-O—C₁₋₈alkylene-CO-**^{L1}, *^{L1}-C₁₋₈alkylene-O-**^{L1}, *^{L1}-C(O)—C₁₋₈-**^{L1}, *^{L1}-C₁₋₈alkylene-C(O)-**^{L1}, *^{L1}-NR^{L1a}—C₁₋₈alkylene-**^{L1}, *^{L1}-C₁₋₈alkylene-NR^{L1a}-**^{L1}, 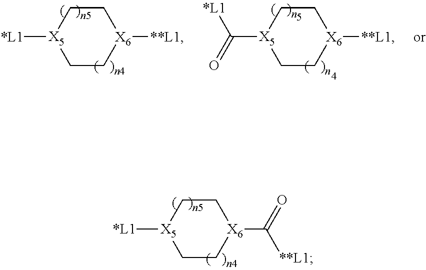
each of said —C₁₋₈alkylene-, *^{L1}-O—C₁₋₈alkylene-**^{L1}, —C₃-C₈cycloalkylene-, *^{L1}-O—C₃-C₈cycloalkylene-**^{L1}, *^{L1}-C₁₋₈alkylene-NR^{L1a}-**^{L1}, *^{L1}-O—C₁₋₈alkylene-CO-**^{L1}, *^{L1}-C₁₋₈alkylene-O-**^{L1}, *^{L1}-C(O)—C₁₋₈alkylene-**^{L1},*^{L1}-C₁₋₈alkylene-C(O)-**^{L1}, *^{L1}-NR^{L1a}—C₁₋₈alkylene-**^{L1},*^{L1}-C₁₋₈alkylene-NR^{L1a}-**^{L1}, 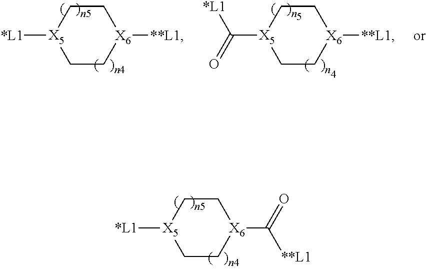
is optionally substituted with at least one R^{L1b}; wherein **^{L1} refers to the position attached to the 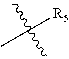
moiety, and *^{L1} refers to the position attached to the other side;
R^{L1a} is selected from hydrogen, —C₁₋₈alkyl, —C₂₋₈alkenyl, —C₂₋₈alkynyl, C₃-C₈cycloalkyl, 3- to 8-membered heterocyclyl, C₆-C₁₂aryl or 5- to 12-membered heteroaryl, each of said —C₁₋₈alkyl, —C₂₋₈alkenyl, —C₂₋₈alkynyl, C₃-C₈cycloalkyl, C₆-C₁₂aryl or 5- to 12-membered heteroaryl is optionally substituted with at least one substituent R^{L1c};
each of said R^{L1b} and R^{L1c} is independently halogen, hydroxy, —C₁₋₈alkyl, —C₁₋₈alkoxy, —C₂₋₈alkenyl, —C₂₋₈alkynyl, C₃-C₈cycloalkyl, 3- to 8-membered heterocyclyl, C₆-C₁₂aryl or 5- to 12-membered heteroaryl; or
R^{L1b} or two R^{L1c} together with the atoms to which they are attached, for a 3- to 6-membered unsaturated or saturated ring, said ring comprising 0-3 heteroatoms independently selected from nitrogen, oxygen or sulfur; said ring is optionally substituted with at least one substituent selected from halogen, hydroxy, —C₁₋₈alkyl, —C₁₋₈alkoxy, —C₂₋₈alkenyl, —C₂₋₈alkynyl, C₃-C₈cycloalkyl, 3- to 8-membered heterocyclyl, C₆-C₁₂aryl or 5- to 12-membered heteroaryl;
each of X₅ and X₆ is CH or N;
n4 and n5 are each independently 0, 1 or 2;
R₅ is hydrogen, halogen, —C₁₋₈alkyl, C₃-C₈cycloalkyl, 3- to 12-membered heterocyclyl, C₆-C₁₂aryl, 5- to 12-membered heteroaryl, oxo, —CN, —OR₅ₐ, —COR₅ₐ, —CO₂R₅ₐ, —CONR₅ₐR_{5b}, —NR₅ₐR_{5b}, —NR₅ₐCOR_{5b} or —NR₅ₐCO₂R_{5b}; wherein each of said —C₁₋₈alkyl, C₃-C₈cycloalkyl, 3- to 12-membered heterocyclyl, C₆-C₁₂aryl or 5- to 12-membered heteroaryl is optionally substituted with at least one substituent R_{5c};
R₅ₐand R_{5b}are each independently selected from hydrogen, —C₁₋₈alkyl, —C₂₋₈alkenyl, —C₂₋₈alkynyl, C₁₋₈alkoxy-C₁₋₈-, C₃-C₈cycloalkyl, 3- to 8-membered heterocyclyl, C₆-C₁₂aryl, 5- to 12-membered heteroaryl or oxo, wherein each of said —C₁₋₈alkyl, —C₂₋₈alkenyl, —C₂₋₈alkynyl, C₁₋₈alkoxy-C₁₋₈alkyl-, C₃-C₈cycloalkyl, 3- to 8-membered heterocyclyl, C₆-C₁₂aryl, 5- to 12-membered heteroaryl is optionally substituted with at least one substituent R_{5d}; or
R₅ₐ and R_{5b} together with the carbon atoms to which they are attached, for a 3- to 8-membered unsaturated or saturated ring, said ring comprising 0-3 heteroatoms independently selected from nitrogen, oxygen or sulfur; said ring is optionally substituted with at least one substituent R_{5c};
R_{5c}, at each occurrence, is independently halogen, —C₁₋₈alkyl, —C₂₋₈alkenyl, —C₂₋₈alkynyl, —C₃-C₈cycloalkyl, 3- to 8-membered heterocyclyl, —C₆-C₁₂aryl, 5- to 12-membered heteroaryl, oxo, —CN, —OR₅ₑ, —COR₅ₑ, —CO₂R₅ₑ, —CONR₅ₑR_{5f}, —NR₅ₑR_{5f}, —NR₅ₑCOR_{5f} or —NR₅ₑCO₂R_{5f}, wherein each of said —C₁₋₈alkyl, —C₂₋₈alkenyl, —C₂₋₈alkynyl, —C₃-C₈cycloalkyl, 3- to 8-membered heterocyclyl, —C₆-C₁₂aryl, or 5- to 12-membered heteroaryl is optionally substituted with at least one substituent R_{5d}; or
two R_{5c} together with the carbon atoms to which they are attached, for a 3- to 8-membered unsaturated or saturated ring, said ring comprising 0-3 heteroatoms independently selected from nitrogen, oxygen or sulfur; said ring is optionally substituted with at least one substituent R_{5d};
R_{5d} is hydrogen, halogen, —C₁₋₈alkyl, C₃-C₈cycloalkyl, 3- to 8-membered heterocyclyl, C₆-C₁₂aryl, 5- to 12-membered heteroaryl, oxo, —CN, —OR_{5g}, —COR_{5g}, —CO₂R_{5g}, —CONR_{5g}R₅ₕ, —NR_{5g}R₅ₕ, —NR_{5g}COR₅ₕ or —NR_{5g}CO₂R₅ₕ; wherein each of said —C₁₋₈alkyl, C₃-C₈cycloalkyl, 3- to 8-membered heterocyclyl, C₆-C₁₂aryl or 5- to 12-membered heteroaryl is optionally substituted with at least one substituent R₅ᵢ;
R₅ₑ, R_{5f}, R_{5g}, R₅ₕ and R₅ᵢ are each independently selected from hydrogen, —C₁₋₈alkyl, —C₁₋₈alkenyl, —C₂₋₈alkynyl, C₁₋₈alkoxy-C₁₋₈alkyl, —C₁-C₈cycloalkyl, 3- to 8-membered heterocyclyl, C₆-C₁₂aryl or 5- to 12-membered heteroaryl.

2. The compound of claim 1, wherein Y₁ is selected from —NH—or —C(R_{Y1a})(NH₂)—.

3. The compound of claim 1, wherein R₁ₐ, R_{1b}, R₂ₐ, R_{2b}, R₃ₐ, R_{3b}, R_{1c}, R_{1d}, R_{2c}, R_{2d}, R_{3c}, R_{3d}, R_{Y1a}, R_{Y1b} and R_{Y2}, if present, are each independently hydrogen, —F, —Cl, —Br, —I, methyl, ethyl, propyl, butyl, pentyl, hexyl, heptyl, octyl, cyclopropyl, cyclobutyl, cyclopentyl, cyclohexyl, cycloheptyl, cyclooctyl, 3- to 8-membered heterocyclyl, phenyl, 5- to 12-membered heteroaryl, —CN, —OR₁ₑ, or —NR₁ₑR_{1f}; wherein each of said methyl, ethyl, propyl, butyl, pentyl, hexyl, heptyl, octyl, cyclopropyl, cyclobutyl, cyclopentyl, cyclohexyl, cycloheptyl, cyclooctyl, 3- to 8-membered heterocyclyl, phenyl or 5- to 12-membered heteroaryl is optionally substituted with at least one substituent R_{1g},
one pair of (R₁ₐ and R_{1c}), (R₁ₐ and R_{2c}), (R₁ₐ and R_{3c}), (R₂ₐ and R_{1c}), (R₂ₐ and R_{2c}), (R₂ₐ and R_{3c}), (R₃ₐ and R_{1c}), (R₃ₐ and R_{2c}), (R₃ₐ and R_{3c}), (R_{Y1a} and R_{Y2}), and (R_{Y1b} and R_{Y2}) form a bridge containing one, two, three, or four —CH₂— moieties in addition to the two bridgeheads, wherein one of the —CH₂— moiety is optionally replaced with —O—, —S—or —NH— and wherein said bridge is optionally substituted with at least one substituent R_{1g};
R₁ₑ and R_{1f} are each independently selected from hydrogen, methyl, ethyl, propyl, butyl, pentyl, hexyl, heptyl, octyl, cyclopropyl, cyclobutyl, cyclopentyl, cyclohexyl, cycloheptyl, cyclooctyl, —C₂₋₈alkenyl, —C₂₋₈alkynyl, C₁₋₈alkoxy-C₁₋₈alkyl-, 3- to 8-membered heterocyclyl, phenyl or 5- to 12-membered heteroaryl;
R_{1g}, at each occurrence, is independently —F, —Cl, —Br, —I, methyl, ethyl, propyl, butyl, pentyl, hexyl, heptyl, octyl, cyclopropyl, cyclobutyl, cyclopentyl, cyclohexyl, cycloheptyl, cyclooctyl, —C₂₋₈alkenyl, —C₂₋₈alkynyl, 3- to 8-membered heterocyclyl, phenyl, or 5- to 12-membered heteroaryl.

4. The compound of claim 1, wherein, the ring 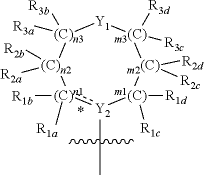
is a bridged bicyclic ring.

5. The compound of claim 1, wherein, the ring 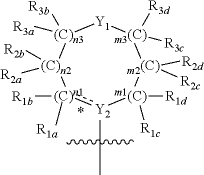
is selected from:
a) Y₁ is NH, Y₂ is N, [symbol] is a single bond, R₃ₐ and R_{3c} form a bridge containing one, two, or three —CH₂— moieties in addition to the two bridgeheads (nitrogen), wherein one of the —CH₂— moiety is optionally replaced with —O—, —S—or —NH— and wherein said bridge is optionally substituted with halogen or alkyl, and n3=m3=n1=m1=1 and n2=m2=0; or
b) Y₁ is NH, Y₂ is N, [symbol] is a single bond, R₂ₐ and R_{2c} form a bridge containing one, two, or three —CH₂— moieties in addition to the two bridgeheads (nitrogen), wherein one of the —CH₂— moiety is optionally replaced with —O—, —S— or —NH—, and n1=m 1=n2=m2=n3=m3=1 ; or
c) Y₁ is NH, Y₂ is N, [symbol] is a single bond, R₁ₐ and R_{1c} form a bridge containing one, two, or three —CH₂— moieties in addition to the two bridgeheads (nitrogen), wherein one of the —CH₂— moiety is optionally replaced with —O—, —S—or —NH—, and n3=m3=n 1=m1=1 and n2=m2=0; or
d) Y₁ is NH, Y₂ is N, [symbol] is a single bond, R₁ₐ and R_{1c} form a bridge containing one, two, or three —CH₂— moieties in addition to the two bridgeheads (nitrogen), wherein one of the —CH₂— moiety is optionally replaced with —O—, —S—or —NH—, and n2=m2=n3=m3=0 and n1=m 1=1; or
e) Y₁ is NH, Y₂ is CH, [symbol] is a single bond, or Y₂ is C, [symbol] is a double bond, R₂ₐ and R_{1c} form a bridge containing one, two, or three —CH₂— moieties in addition to the two bridgeheads (one nitrogen and one carbon atom), wherein one of the —CH₂— moiety is optionally replaced with —O—, —S—or —NH—, and n1=n2=n3=m1=1, and m2=m3=0; or
f) Y₁ is NH, Y₂ is CH, [symbol] is a single bond, or Y₂ is C, [symbol] is a double bond, R₃ₐ and R_{3c} form a bridge containing one, two, or three —CH₂— moieties in addition to the two bridgeheads (one nitrogen and one carbon atom), wherein one of the —CH₂— moiety is optionally replaced with —O—, —S— or —NH—, and n1=m1=n3=m3=1, and n2=m2=0; or n3=m3=m1=1, and n1=n2=m2=0; or
g) Y₁ is CH, Y₂ is CH, [symbol] is a single bond, R_{Y1a} and R_{Y2} form a bridge containing one, two, or three —CH₂— moieties in addition to the two bridgeheads (two carbon atoms), wherein one of the —CH₂— moiety is optionally replaced with —O—, —S— or —NH—, and n1=m1=n3=m3=0, and n2=m2=1; or
h) Y₁ is NH, Y₂ is N, [symbol] is a single bond, R₃ₐ and R_{1c} form a bridge containing one, two, or three —CH₂— moieties in addition to the two bridgeheads (nitrogen), wherein one of the —CH₂— moiety is optionally replaced with —O—, —S— or —NH—, n1=m1=n3=m3=1, and n2=m2=0; or
i) Y₁ is NH, Y₂ is —CH(NH₂), [symbol] is a single bond, R₁ₐ and R_{1c} form a bridge containing one, two, or three —CH₂— moieties in addition to the two bridgeheads (one nitrogen and one carbon atom), wherein one of the —CH₂— moiety is optionally replaced with —O—, —S— or —NH—, n1=m1=n3=1, and n2=m2=m3=0.

6. The compound of claim 5, wherein the 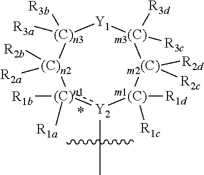
moiety is selected from

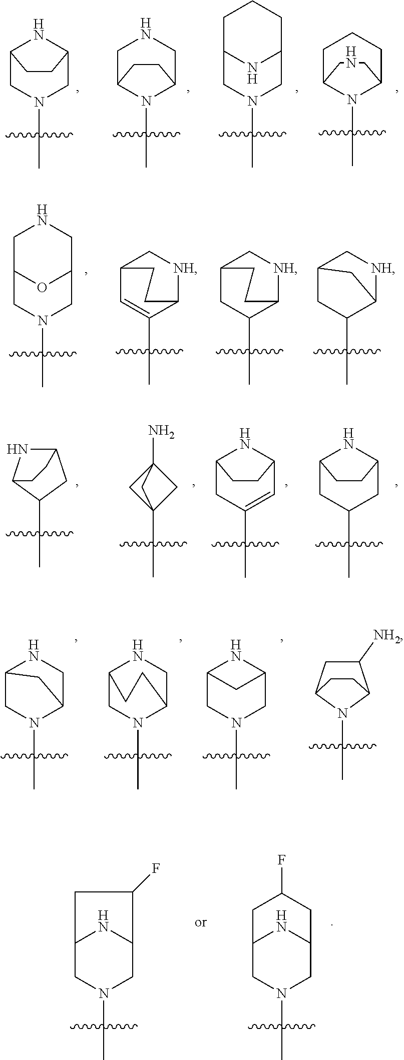

7. The compound claim 1, wherein R₆ is hydrogen, —F, —Cl, —Br, —I, methyl, ethyl, propyl, butyl, pentyl, hexyl, heptyl, octyl, vinyl, propylenyl, allyl, butenyl, butadienyl, pentenyl, pentadienyl, hexenyl, hexadienyl, ethynyl, propynyl, butynyl, pentynyl, hexynyl, cyclopropyl, cyclobutyl, cyclopentyl, cyclohexyl, cycloheptyl, cyclooctyl, oxazolidinyl, imidazolidinyl, thiazolidinyl, pyrazolidinyl, morpholinyl, piperidinyl, piperazinyl, oxazinyl, imidazolyl, thiazolyl, oxazolyl, pyridyl, pyrimidinyl, pyrazinyl, pyridazinyl, phenyl, pyrrolyl, imidazolyl, pyrazolyl, thiazolyl, oxazolyl, triazolyl, thiophenyl, furanyl, pyridyl, pyrimidinyl, pyrazinyl, —CN, oxo, —OR₆ₐ, —SR₆ₐ or —NR₆ₐR_{6b}; each of said methyl, ethyl, propyl, butyl, pentyl, hexyl, heptyl, octyl, cyclopropyl, cyclobutyl, cyclopentyl, cyclohexyl, cycloheptyl, cyclooctyl, oxazolidinyl, imidazolidinyl, thiazolidinyl, pyrazolidinyl, morpholinyl, piperidinyl, piperazinyl, oxazinyl, imidazolyl, thiazolyl, oxazolyl, pyridyl, pyrimidinyl, pyrazinyl, pyridazinyl, phenyl, pyrrolyl, imidazolyl, pyrazolyl, thiazolyl, oxazolyl, triazolyl, thiophenyl, furanyl, pyridyl, pyrimidinyl or pyrazinyl is optionally substituted with at least one substituent R_{6c},
R₆ₐ and R_{6b}are each independently selected from hydrogen, methyl, ethyl, propyl, butyl, pentyl, hexyl, heptyl, octyl, —C₂₋₈alkenyl, —C₂₋₈alkynyl, C₁₋₈alkoxy-C₁₋₈alkyl-, cyclopropyl, cyclobutyl, cyclopentyl, cyclohexyl, cycloheptyl, cyclooctyl, 3- to 8-membered heterocyclyl, phenyl or 5- to 12-membered heteroaryl; wherein each of said methyl, ethyl, propyl, butyl, pentyl, hexyl, heptyl, octyl, —C₂₋₈alkenyl, —C₂₋₈alkynyl, C₁₋₈alkoxy-C₁₋₈alkyl-, cyclopropyl, cyclobutyl, cyclopentyl, cyclohexyl, cycloheptyl, cyclooctyl, 3- to 8-membered heterocyclyl, phenyl or 5- to 12-membered heteroaryl is optionally substituted with at least one substituent R_{6d};
R_{6c}, at each occurrence, is independently —F, —Cl, —Br, —I, methyl, ethyl, propyl, butyl, pentyl, hexyl, heptyl, octyl, —C₂₋₈alkenyl, —C₂₋₈alkynyl, cyclopropyl, cyclobutyl, cyclopentyl, cyclohexyl, cycloheptyl, cyclooctyl, 3- to 8-membered heterocyclyl, phenyl, or 5- to 12-membered heteroaryl; or two R₆ together with the atoms to which they are attached, form a 5- to 6-membered unsaturated- or saturated ring, said ring comprising 0, 1, 2, or 3 heteroatoms independently selected from nitrogen, oxygen or sulfur; said ring is optionally substituted with at least one substituent R_{6d};
R_{6d} is hydrogen, —F, —Cl, —Br, —I, methyl, ethyl, propyl, butyl, pentyl, hexyl, heptyl, octyl, cyclopropyl, cyclobutyl, cyclopentyl, cyclohexyl, cycloheptyl, cyclooctyl, vinyl, propylenyl, allyl, butenyl, butadienyl, pentenyl, pentadienyl, hexenyl, hexadienyl, ethynyl, propynyl, butynyl, pentynyl, hexynyl, 3- to 8-membered heterocyclyl, phenyl, 5- to 12-membered heteroaryl, —CN, oxo, —OR₆ₑ or —NR₆ₑR_{6f}; each of said methyl, ethyl, propyl, butyl, pentyl, hexyl, heptyl, octyl, cyclopropyl, cyclobutyl, cyclopentyl, cyclohexyl, cycloheptyl, cyclooctyl, vinyl, propylenyl, allyl, butenyl, butadienyl, pentenyl, pentadienyl, hexenyl, hexadienyl, ethynyl, propynyl, butynyl, pentynyl, hexynyl, 3- to 8-membered heterocyclyl, phenyl or 5- to 12-membered heteroaryl is optionally substituted with at least one substituent R_{6g},
R_{6c} and R_{6f} are each independently selected from hydrogen, methyl, ethyl, propyl, butyl, pentyl, hexyl, heptyl, octyl, —C₂₋₈alkenyl, —C₂₋₈alkynyl, —C₁₋₈alkoxy-C₁₋₈alkyl-, cyclopropyl, cyclobutyl, cyclopentyl, cyclohexyl, cycloheptyl, cyclooctyl, 3- to 8-membered heterocyclyl, phenyl or 5- to 12-membered heteroaryl;
R_{6g}, at each occurrence, is independently —F, —Cl, —Br, —I, methyl, ethyl, propyl, butyl, pentyl, hexyl, heptyl, octyl, cyclopropyl, cyclobutyl, cyclopentyl, cyclohexyl, cycloheptyl, cyclooctyl, —C₂₋₈alkenyl, —C₂₋₈alkynyl, 3- to 8-membered heterocyclyl, phenyl, or 5- to 12-membered heteroaryl.

8. The compound of claim 1, wherein Ring A is an aryl group selected from phenyl or naphthyl substituted with one or two R₆.

9. The compound of claim 1, wherein Ring A is a 5- to 7-membered monocyclic heteroaryl or 8- to 12-membered bicyclic heteroaryl group substituted with one or two R₆.

10. The compound of claim 1, wherein the 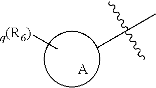
moiety is

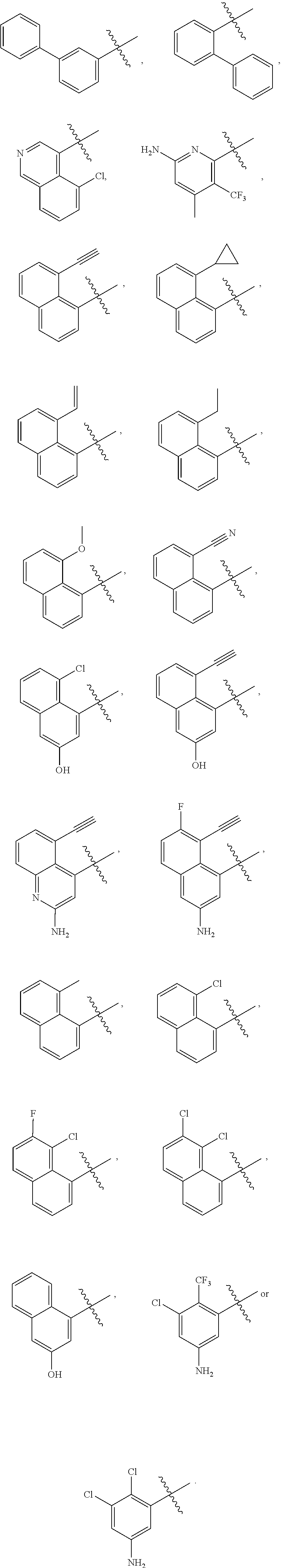

11. The compound of claim 1, wherein R₄ is hydrogen, —F, —Cl, —Br, —I, methyl, ethyl, propyl, butyl, pentyl, hexyl, heptyl, cyclopropyl, cyclobutyl, cyclopentyl, cyclohexyl, cycloheptyl, cyclooctyl, vinyl, propylenyl, allyl, butenyl, butadienyl, pentenyl, pentadienyl, hexenyl, hexadienyl, ethynyl, propynyl, butynyl, pentynyl, hexynyl, 3- to 8-membered heterocyclyl, phenyl, 5- to 12-membered heteroaryl, —CN, oxo, —OR₄ₐ, —SR₄ₐ, —SO₂R₄ₐ, —SO₂NR₄ₐR_{4b}, —COR₄ₐ, —CO₂R₄ₐ, —CONR₄ₐR_{4b}, —NR₄ₐR_{4b}, —NR₄ₐCOR_{4b}, —NR₄ₐCO₂R_{4b}, or —NR₄ₐSO₂R_{4b}; each of said methyl, ethyl, propyl, butyl, pentyl, hexyl, heptyl, cyclopropyl, cyclobutyl, cyclopentyl, cyclohexyl, cycloheptyl, cyclooctyl, vinyl, propylenyl, allyl, butenyl, butadienyl, pentenyl, pentadienyl, hexenyl, hexadienyl, ethynyl, propynyl, butynyl, pentynyl, hexynyl, 3- to 8-membered heterocyclyl, phenyl or 5- to 12-membered heteroaryl is optionally substituted with at least one substituent R_{4c},
R₄ₐ and R_{4b} are each independently selected from hydrogen, methyl, ethyl, propyl, butyl, pentyl, hexyl, heptyl, cyclopropyl, cyclobutyl, cyclopentyl, cyclohexyl, cycloheptyl, cyclooctyl, vinyl, propylenyl, allyl, butenyl, butadienyl, pentenyl, pentadienyl, hexenyl, hexadienyl, ethynyl, propynyl, butynyl, pentynyl, hexynyl, C₁₋₈alkoxy-C₁₋₈alkyl-, 3- to 8-membered heterocyclyl, phenyl or 5- to 12-membered heteroaryl; each of said methyl, ethyl, propyl, butyl, pentyl, hexyl, heptyl, cyclopropyl, cyclobutyl, cyclopentyl, cyclohexyl, cycloheptyl, cyclooctyl, vinyl, propylenyl, allyl, butenyl, butadienyl, pentenyl, pentadienyl, hexenyl, hexadienyl, ethynyl, propynyl, butynyl, pentynyl, hexynyl, C₁₋₈alkoxy-C₁₋₈alkyl-, 3- to 8-membered heterocyclyl, phenyl or 5- to 12-membered heteroaryl is optionally substituted with at least one substituent R_{4d};
R_{4c} and R_{4d}, at each occurrence, are each independently —F, —Cl, —Br, —I, methyl, ethyl, propyl, butyl, pentyl, hexyl, heptyl, vinyl, propylenyl, allyl, butenyl, butadienyl, pentenyl, pentadienyl, hexenyl, hexadienyl, ethynyl, propynyl, butynyl, pentynyl, hexynyl, cyclopropyl, cyclobutyl, cyclopentyl, cyclohexyl, cycloheptyl, cyclooctyl, 3- to 8-membered heterocyclyl, —C₆-C₁₂aryl, or 5- to 12-membered heteroaryl.

12. The compound of claim 1, wherein R₄ is hydrogen, —F, —Cl, —Br, —I, methyl, ethyl, propyl, isopropyl, isobutyl, tert-butyl, —CF₃, —CHF₂, —CH₂F, —OCHF₂, —OCF₃, cyclopropyl, cyclobutyl, cyclopentyl, vinyl, propylenyl or allyl.

13. The compound of claim 1, wherein L₁ is selected from a single bond, —O—, —NR_{L1a}, —C(O)—, —CH₂—, —CH₂CH₂—, —CH₂CH₂CH₂—, —CH₂CH₂CH₂CH₂—, —CH₂CH₂CH₂CH₂CH₂—, —CH₂CH₂CH₂CH₂CH₂CH₂—, —CH₂CH₂CH₂CH₂CH₂CH₂CH₂—, —CH₂CH₂CH₂CH₂CH₂CH₂CH₂CH₂—, *^{L1}-O—CH₂-**^{L1}, *^{L1}-O—CH₂CH₂-**^{L1}, *^{L1}-O—CH₂CH₂CH₂- **^{L1}, *^{L1}-O—CH₂CH₂CH₂CH₂-**^{L1}, *^{L1}-O—CH₂CH₂CH₂CH₂CH₂-**^{L1}, *^{L1}-O—CH₂CH₂CH₂CH₂CH₂CH₂- **^{L1}, *^{L1}-O—CH₂CH₂CH₂CH₂CH₂CH₂CH₂-**^{L1}, *^{L1}-O—CH₂CH₂CH₂CH₂CH₂CH₂CH₂CH₂-**^{L1}, —C₃₋₈cycloalkylene-, *^{L1}-O—C₃-C₈cycloalkylene-**^{L1}, *^{L1}-O—CH₂—NR^{L1a}-**^{L1}, *^{L1}-O—CH₂CH₂—NR^{L1}-**^{L1}, *^{L1}-O—CH₂CH₂CH₂—NR^{L1a}-**^{L1}, *^{L1}-O—CH₂CH₂CH₂CH₂—NR^{L1a}-**^{L1}, *^{L1}-O—CH₂CH₂CH₂CH₂CH₂—NR^{L1a}-**^{L1}, *^{L1}-O—CH₂CH₂CH₂CH₂CH₂CH₂—NR^{L1a}-**^{L1}, *^{L1}-O—CH₂CH₂CH₂CH₂CH₂CH₂CH₂—NR^{L1a}-**^{L1}, *^{L1}-O—CH₂CH₂CH₂CH₂CH₂CH₂CH₂CH₂—NR^{L1a}- **^{L1}, *^{L1}-O—CH₂—CO-**^{L1}, *^{L1}-O—CH₂CH₂—CO-**^{L1}, *^{L1}-O—CH₂CH₂CH₂—CO-**^{L1}, *^{L1}-O—CH₂CH₂CH₂CH₂—CO-**^{L1}, *^{L1}-O—CH₂CH₂CH₂CH₂CH₂—CO-**^{L1}, *^{L1}-O—CH₂CH₂CH₂CH₂CH₂CH₂—CO-**^{L1}, *^{L1}-O—CH₂CH₂CH₂CH₂CH₂CH₂CH₂—CO -**^{L1}, *^{L1}-CH₂O-**^{L1}, *^{L1}-CH₂CH₂—O-**^{L1}, *^{L1}-CH₂CH₂CH₂—O-**^{L1}, *^{L1}-CH₂CH₂CH₂CH₂—O-**^{L1}, *^{L1}-CH₂CH₂CH₂CH₂CH₂—O-**^{L1}, *^{L1}-CH₂CH₂CH₂CH₂CH₂CH₂—O-**^{L1}, *^{L1}-CH₂CH₂CH₂CH₂CH₂CH₂CH₂—O-**^{L1}, *^{L1}-CH₂CH₂CH₂CH₂CH₂CH₂CH₂CH₂—O-**^{L1}, *^{L1}—C(O)—CH₂-**^{L1}, *^{L1}-C(O)—CH₂CH₂-*^{L1}, *^{L1}-C(O)—CH₂CH₂CH₂-**^{L1}, *^{L1}-C(O)—CH₂CH₂CH₂CH₂-**^{L1}, *^{L1}-C(O)—CH₂CH₂CH₂CH₂CH₂-**^{L1}, *^{L1}-C(O)—CH₂CH₂CH₂CH₂CH₂CH₂-**^{L1}, *^{L1}-C(O)—CH₂CH₂CH₂CH₂CH₂CH₂CH₂-**^{L1}, *^{L1}-C(O)—CH₂CH₂CH₂CH₂CH₂CH₂CH₂CH₂-**^{L1}, *^{L1}-CH₂—C(O)-**^{L1}, *^{L1}-CH₂CH₂—C(O)-**^{L1}, -CH₂CH₂CH₂—C(O)-**^{L1}, *^{L1}CH₂CH₂CH₂CH₂—C(O)-**^{L1}, *^{L1}-CH₂CH₂CH₂CH₂CH₂—C(O)-**^{L1}, *^{L1}-CH₂CH₂CH₂CH₂CH₂CH₂—C(O)-**^{L1}, *^{L1}CH₂CH₂CH₂CH₂CH₂CH₂CH₂—C(O)-**^{L1}, *^{L1}-CH₂CH₂CH₂CH₂CH₂CH₂CH₂CH₂—C(O)-**^{L1}, *^{L1}-CH₂—NR^{L1a}-**^{L1}, *^{L1}-CH₂CH₂—NR^{L1a}-**^{L1}, *^{L1}-CH₂CH₂CH₂—NR^{L1a}-**^{L1}, *^{L1}-CH₂CH₂CH₂CH₂—NR^{L1a}-**^{L1}, *^{L1}-CH₂CH₂CH₂CH₂CH₂—NR^{L1a}-**^{L1}, *^{L1}-CH₂CH₂CH₂CH₂CH₂CH₂—NR^{L1a}-**^{L1}, *^{L1}-CH₂CH₂CH₂CH₂CH₂CH₂CH₂—NR^{L1a}-**^{L1}, *^{L1}-CH₂CH₂CH₂CH₂CH₂CH₂CH₂CH₂—NR^{L1a}-**^{L1}, **^{L1}-NR^{L1a}—CH₂-**^{L1}, *^{L1}-NR^{L1}—CH₂CH₂-**^{L1}, *^{L1}-NR^{L1a}—CH₂CH₂CH₂-**^{L1}, *^{L1}-NR^{L1a}—CH₂CH₂CH₂CH₂-**^{L1}, *^{L1}-NR^{L1a}—CH₂CH₂CH₂CH₂CH₂-**^{L1}, *^{L1}-NR^{L1a}—CH₂CH₂CH₂CH₂CH₂CH₂-**^{L1}, *^{L1}-NR^{L1a}—CH₂CH₂CH₂CH₂CH₂CH₂CH₂-**^{L1}, *^{L1}-NR^{L1a}—CH₂CH₂CH₂CH₂CH₂CH₂CH₂CH₂-**^{L1}, 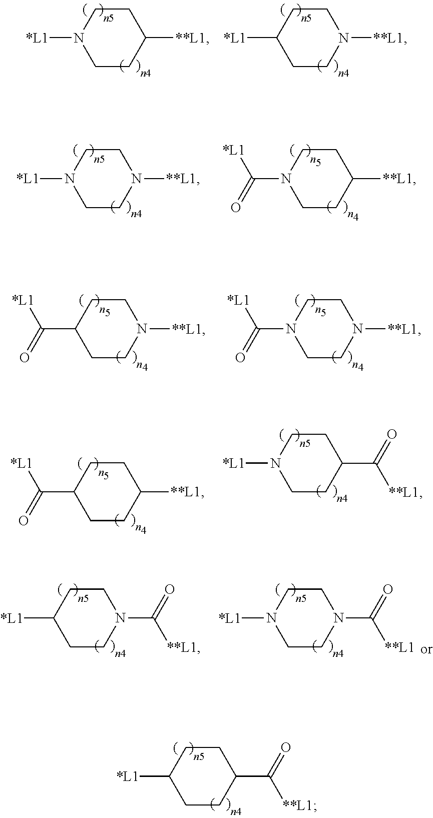
each of said —CH₂—, —CH₂CH₂—, —CH₂CH₂CH₂—, δCH₂CH₂CH₂CH₂δ, —CH₂CH₂CH₂CH₂CH₂—, —CH₂CH₂CH₂CH₂CH₂CH₂—, —CH₂CH₂CH₂CH₂CH₂CH₂CH₂—, —CH₂CH₂CH₂CH₂CH₂CH₂CH₂CH₂—, *^{L1}-O—CH₂-**^{L1}, *^{L1}-O—CH₂CH₂-**^{L1}, *^{L1}-O—CH₂CH₂CH₂-**^{L1}, *^{L1}—O—CH₂CH₂CH₂CH₂-**^{L1}, *^{L1}-O—CH₂CH₂CH₂CH₂CH₂-**^{L1}, *^{L1}-O—CH₂CH₂CH₂CH₂CH₂CH₂-**^{L1}-O—CH₂CH₂CH₂CH₂CH₂CH₂CH₂-**^{L1}, *^{L1}-O—CH₂CH₂CH₂CH₂CH₂CH₂CH₂CH₂-**^{L1}, —C₃-C₈cycloalkylene-, *^{L1}-O—C₃-C₈cycloalkylene-**^{L1}, *^{L1}-O—CH₂—NR^{L1a}-**^{L1}, *^{L1}-OCH₂CH₂—NR^{L1a}-**^{L1}, *^{L1}-O—CH₂CH₂CH₂—NR^{L1a}-**^{L1}, *^{L1}-O—CH₂CH₂CH₂CH₂——NR^{L1a}-**^{L1}, *^{L1}-O—CH₂CH₂CH₂CH₂CH₂—NR^{L1a}-**^{L1}, *^{L1}-O—CH₂CH₂CH₂CH₂CH₂CH₂-NR^{L1a}-**^{L1}, -O—CH₂CH₂CH₂CH₂CH₂CH₂CH₂—NR^{L1a}-**^{L1}, *^{L1}-O—CH₂CH₂CH₂CH₂CH₂CH₂CH₂CH₂—NR^{L1a}-**^{L1}, *^{L1}-O—CH₂—CO-**^{L1}, *^{L1}-O—CH₂CH₂—CO-**^{L1}, *^{L1}-O—CH₂CH₂CH₂—CO-**^{L1}, *^{L1}-O—CH₂CH₂CH₂CH₂—CO-**^{L1}, *^{L1}-O—CH₂CH₂CH₂CH₂CH₂—CO-**^{L1}, *^{L1}-O—CH₂CH₂CH₂CH₂CH₂CH₂—CO-**^{L1}, *^{L1}-O—CH₂CH₂CH₂CH₂CH₂CH₂CH₂—CO-**^{L1}, *^{L1}-O—CH₂CH₂CH₂CH₂CH₂CH₂CH₂CH₂—CO-**^{L1}, *^{L1}-CH₂—O-**^{L1}, *^{L1}-CH₂CH₂—O-**^{L1}, *^{L1}-CH₂CH₂CH₂—O-**^{L1}, *^{L1}-CH₂CH₂CH₂CH₂—O-**^{L1}, *^{L1}-CH₂CH₂CH₂CH₂CH₂—O-**^{L1}, *^{L1}-CH₂CH₂CH₂CH₂CH₂CH₂—O-**^{L1}, *^{L1}-CH₂CH₂CH₂CH₂CH₂CH₂CH₂—O-**^{L1}, *^{L1}-CH₂CH₂CH₂CH₂CH₂CH₂CH₂CH₂—O-**^{L1}, *^{L1}-C(O)—CH₂-**^{L1}, *^{L1}-C(O)—CH₂CH₂-**^{L1}, *^{L1}-C(O)—CH₂CH₂CH₂-**^{L1}, *^{L1}-C(O)—CH₂CH₂CH₂CH₂-**^{L1}, *^{L1}-C(O)—CH₂CH₂CH₂CH₂CH₂-**^{L1}, *^{L1}-C(O)—CH₂CH₂CH₂CH₂CH₂CH₂-**^{L1}, *^{L1}C(O)—CH₂CH₂CH₂CH₂CH₂CH₂CH₂-**^{L1}, *^{L1}-C(O)—CH₂CH₂CH₂CH₂CH₂CH₂CH₂CH₂-**^{L1}, *^{L1}—CH₂—C(O)-**^{L1}, *^{L1}-CH₂CH₂—C(O)-**^{L1}, *^{L1}-CH₂CH₂CH₂—C(O)-**^{L1}, *^{L1}-CH₂CH₂CH₂CH₂—C(O)-**^{L1}, *^{L1}CH₂CH₂CH₂CH₂CH₂—C(O)-**^{L1}, *^{L1}-CH₂CH₂CH₂CH₂CH₂CH₂—C(O)-**^{L1}, *^{L1}-CH₂CH₂CH₂CH₂CH₂CH₂CH₂—C(O)-**^{L1}, *^{L1}-CH₂CH₂CH₂CH₂CH₂CH₂CH₂CH₂—C(O)-**^{L1}, *^{L1}CH₂—NR^{L1a}-**^{L1}, *^{L1}CH₂CH₂—NR^{L1a}-**^{L1}, *^{L1}-CH₂CH₂CH₂—NR^{L1a}-**^{L1}, *^{L1}CH₂CH₂CH₂CH₂—NR^{L1a}-**^{L1}, *^{L1}-CH₂CH₂CH₂CH₂CH₂—NR^{L1a}- **^{L1}, *^{L1}-CH₂CH₂CH₂CH₂CH₂CH₂—NR^{L1a}-**^{L1}, *^{L1}CH₂CH₂CH₂CH₂CH₂CH₂CH₂—NR^{L1a}-**^{L1}, *^{L1}-CH₂CH₂CH₂CH₂CH₂CH₂CH₂CH₂—NR^{L1a}-**^{L1}, *^{L1}-NR^{L1a}—CH₂—**^{L1}, *^{L1}-NR^{L1a}—CH₂CH₂-**^{L1}, *^{L1}-CH₂CH₂CH₂-**^{L1}, *L₁-NR^{L1a}—CH₂CH₂CH₂CH₂-**^{L1}, *L₁-NR^{L1a}—CH₂CH₂CH₂CH₂CH₂-**^{L1}, *^{L1}-CH₂CH₂CH₂CH₂CH₂CH₂-**^{L1}, *^{L1}-NR^{L1a}—CH₂CH₂CH₂CH₂CH₂CH₂CH₂-**^{L1}, *^{L1}-NR^{L1a}—CH₂CH₂CH₂CH₂CH₂CH₂CH₂CH₂-**^{L1}, 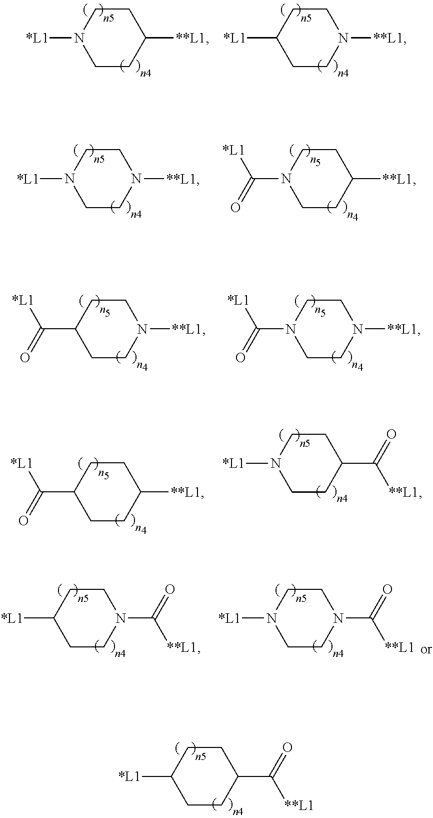
is optionally substituted with at least one R^{L1b};
n4 and n5 are each independently 0 or 1;
R^{L1a} is selected from hydrogen, —C₁₋₈alkyl, —C₂₋₈alkenyl, —C₂₋₈alkynyl, C₃-C₈cycloalkyl, 3- to 8-membered heterocyclyl, C₆-C₁₂aryl or 5- to 12-membered heteroaryl, each of said —C₁₋₈alkyl, —C₂₋₈alkenyl, —C₂₋₈alkynyl, C₃-C₈cycloalkyl, 3- to 8-membered heterocyclyl, C₆-C₁₂aryl or 5- to 12-membered heteroaryl is optionally substituted with at least one substituent R^{L1c};
each of said R^{L1b} and R^{L1c} are independently halogen, hydroxy, —C₁₋₈alkyl, 13 C₁₋₈alkoxy, —C₂₋₈alkenyl, —C₂₋₈alkynyl, C₃-C₈cycloalkyl, 3- to 8-membered heterocyclyl, C₆-C₁₂aryl or 5- to 12-membered heteroaryl; or
two R^{L1b} or two R^{L1c} together with the atoms to which they are attached, form a 3- to 6-membered unsaturated or saturated ring, said ring comprising 0-3 heteroatoms independently selected from nitrogen, oxygen or sulfur; said ring is optionally substituted with at least one substituent halogen, hydroxy, —C₁₋₈alkyl, —C₁₋₈alkoxy, —C₂₋₈alkenyl, —C₂₋₈alkynyl, C₃-C₈cycloalkyl, 3- to 8-membered heterocyclyl, C₆-C₁₂aryl or 5- to 12-membered heteroaryl.

14. The compound of claim 1, wherein L₁ is selected from 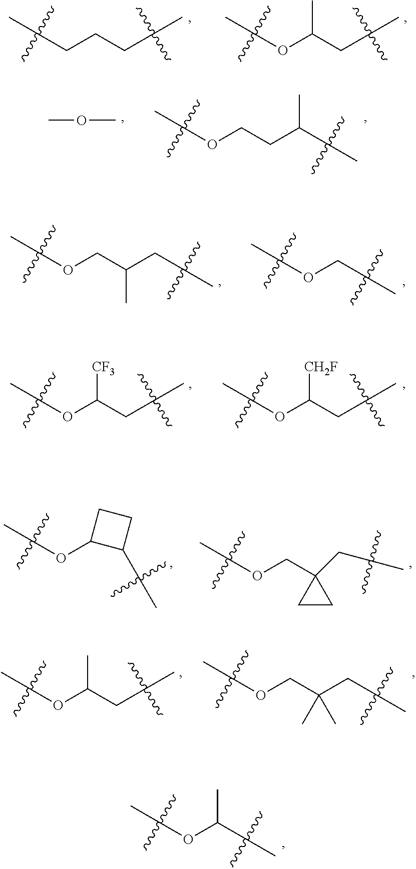
a single bond,

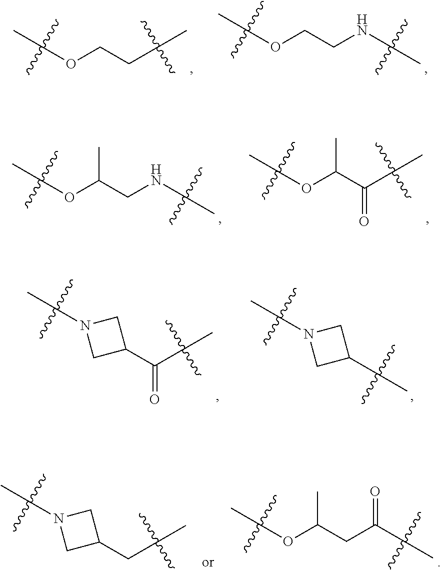

15. The compound of claim 1, wherein R₅ is hydrogen, —F, —Cl, —Br, —I, methyl, ethyl, propyl, butyl, pentyl, hexyl, heptyl, octyl, cyclopropyl, cyclobutyl, cyclopentyl, cyclohexyl, cycloheptyl, cyclooctyl, tetrahydrofuranyl, tetrahydropyranyl, 7- to 9-membered spiro-heterocylic ring comprising one or two or three nitrogen atoms as the ring members oxazolidinyl, imidazolidinyl, thiazolidinyl, pyrazolidinyl, morpholinyl, piperidinyl, piperazinyl, oxazinyl, imidazolyl, thiazolyl, oxazolyl, pyridyl, pyrimidinyl, pyrazinyl, pyridazinyl, phenyl, pyrrolyl, imidazolyl, pyrazolyl, thiazolyl, oxazolyl, triazolyl, thiophenyl, furanyl, pyridyl, pyrimidinyl, pyrazinyl, phenyl, pyrrolidinyl, octahydroquinolizinyl, hexahydro-1H-pyrrolizinyl, oxo, —CN, —OR₅ₐ, —COR₅ₐ, —CO₂R₅ₐ, —CONR₅ₐR_{5b}, —NR₅ₐR_{5b}, —NR₅ₐCOR_{5b} or —NR₅ₐCO₂R₅b; wherein each of said methyl, ethyl, propyl, butyl, pentyl, hexyl, heptyl, octyl, cyclopropyl, cyclobutyl, cyclopentyl, cyclohexyl, cycloheptyl, cyclooctyl, tetrahydrofuranyl, tetrahydropyranyl, 7- to 9-membered spiro-heterocylic ring comprising one or two or three nitrogen atoms as the ring members, oxazolidinyl, imidazolidinyl, thiazolidinyl, pyrazolidinyl, morpholinyl, piperidinyl, piperazinyl, oxazinyl, imidazolyl, thiazolyl, oxazolyl, pyridyl, pyrimidinyl, pyrazinyl, pyridazinyl, phenyl, pyrrolyl, imidazolyl, pyrazolyl, thiazolyl, oxazolyl, triazolyl, thiophenyl, furanyl, pyridyl, pyrimidinyl, pyrazinyl or phenyl is optionally substituted with at least one substituent R_{5c} ;
R₅ₐ and R_{5b} are each independently selected from hydrogen, methyl, ethyl, propyl, butyl, pentyl, hexyl, heptyl, —C₂₋₈alkenyl, —C₂₋₈alkynyl, C₁₋₈alkoxy-C₁₋₈alkyl-, octyl, cyclopropyl, cyclobutyl, cyclopentyl, cyclohexyl, cycloheptyl, cyclooctyl, oxazolidinyl, imidazolidinyl, thiazolidinyl, pyrazolidinyl, morpholinyl, piperidinyl, piperazinyl, oxazinyl, imidazolyl, thiazolyl, oxazolyl, pyridyl, pyrimidinyl, pyrazinyl, pyridazinyl, phenyl, pyrrolyl, imidazolyl, pyrazolyl, thiazolyl, oxazolyl, triazolyl, thiophenyl, furanyl, pyridyl, pyrimidinyl, pyrazinyl or oxo; wherein each of said methyl, ethyl, propyl, butyl, pentyl, hexyl, heptyl, —C₂₋₈alkenyl, —C₂₋₈alkynyl, C₁₋₈alkoxy-C₁₋₈alkyl-, octyl, cyclopropyl, cyclobutyl, cyclopentyl, cyclohexyl, cycloheptyl, cyclooctyl, oxazolidinyl, imidazolidinyl, thiazolidinyl, pyrazolidinyl, morpholinyl, piperidinyl, piperazinyl, oxazinyl, imidazolyl, thiazolyl, oxazolyl, pyridyl, pyrimidinyl, pyrazinyl, pyridazinyl, phenyl, pyrrolyl, imidazolyl, pyrazolyl, thiazolyl, oxazolyl, triazolyl, thiophenyl, furanyl, pyridyl, pyrimidinyl, pyrazinyl is optionally substituted with at least one substituent R_{5d}; or R₅ₐ and R_{5b} together with the carbon atoms to which they are attached, form a 3- to 8-membered unsaturated or saturated ring, said ring comprising 0, 1, 2, or 3 heteroatoms independently selected from nitrogen, oxygen or sulfur; said ring is optionally substituted with at least one substituent R_{5c};
R_{5c}, at each occurrence, is independently halogen, —F, —Cl, —Br, —I, methyl, ethyl, propyl, butyl, pentyl, hexyl, heptyl, octyl, cyclopropyl, cyclobutyl, cyclopentyl, cyclohexyl, cycloheptyl, cyclooctyl, oxazolidinyl, imidazolidinyl, thiazolidinyl, pyrazolidinyl, morpholinyl, piperidinyl, piperazinyl, oxazinyl, imidazolyl, thiazolyl, oxazolyl, pyridyl, pyrimidinyl, pyrazinyl, pyridazinyl, phenyl, pyrrolyl, imidazolyl, pyrazolyl, thiazolyl, oxazolyl, triazolyl, thiophenyl, furanyl, pyridyl, pyrimidinyl, pyrazinyl, —C₂₋₈alkenyl, —C₂₋₈alkynyl, oxo, —CN, —OR₅ₑ, —COR₅ₑ, —CO₂R₅ₑ, —CONR₅ₑR_{5f}, —NR₅ₑR_{5f}, —NR₅ₑCOR_{5f} or —NR₅ₑCO₂R_{5f}; or
two R_{5c} together with the carbon atoms to which they are attached, form a 3- to 8-membered unsaturated or saturated ring, said ring comprising 0, 1, 2, or 3 heteroatoms independently selected from nitrogen, oxygen or sulfur; said ring is optionally substituted with at least one substituent R_{5d};
R_{5d} is hydrogen, —F, —Cl, —Br, —I, methyl, ethyl, propyl, butyl, pentyl, hexyl, heptyl, octyl, cyclopropyl, cyclobutyl, cyclopentyl, cyclohexyl, cycloheptyl, cyclooctyl, oxazolidinyl, imidazolidinyl, thiazolidinyl, pyrazolidinyl, morpholinyl, piperidinyl, piperazinyl, oxazinyl, imidazolyl, thiazolyl, oxazolyl, pyridyl, pyrimidinyl, pyrazinyl, pyridazinyl, phenyl, pyrrolyl, imidazolyl, pyrazolyl, thiazolyl, oxazolyl, triazolyl, thiophenyl, furanyl, pyridyl, pyrimidinyl, pyrazinyl, oxo, —CN, —OR_{5g}, —COR_{5g}, —CO₂R_{5g}, —CONR_{5g}R₅ₕ, —NR_{5g}R_{5g}, —NR_{5g}COR₅ₕ or —NR_{5g}CO₂R₅ₕ; wherein each of said methyl, ethyl, propyl, butyl, pentyl, hexyl, heptyl, octyl, cyclopropyl, cyclobutyl, cyclopentyl, cyclohexyl, cycloheptyl, cyclooctyl, oxazolidinyl, imidazolidinyl, thiazolidinyl, pyrazolidinyl, morpholinyl, piperidinyl, piperazinyl, oxazinyl, imidazolyl, thiazolyl, oxazolyl, pyridyl, pyrimidinyl, pyrazinyl, pyridazinyl, phenyl, pyrrolyl, imidazolyl, pyrazolyl, thiazolyl, oxazolyl, triazolyl, thiophenyl, furanyl, pyridyl, pyrimidinyl or pyrazinyl is optionally substituted with at least one substituent R₅ᵢ;
R₅ₑ, R_{5f}, R_{5g}, R₅ₕ and R₅ᵢ are each independently selected from hydrogen, methyl, ethyl, propyl, butyl, pentyl, hexyl, heptyl, octyl, cyclopropyl, cyclobutyl, cyclopentyl, cyclohexyl, cycloheptyl, cyclooctyl, oxazolidinyl, imidazolidinyl, thiazolidinyl, pyrazolidinyl, morpholinyl, piperidinyl, piperazinyl, oxazinyl, imidazolyl, thiazolyl, oxazolyl, pyridyl, pyrimidinyl, pyrazinyl, pyridazinyl, phenyl, pyrrolyl, imidazolyl, pyrazolyl, thiazolyl, oxazolyl, triazolyl, thiophenyl, furanyl, pyridyl, pyrimidinyl, pyrazinyl, —C₂₋₈alkenyl, —C₂₋₈alkynyl, C₁₋₈alkoxy-C₁₋₈alkyl- or C₃-C₈cycloalkyl.

16. The compound of claim 1, wherein R₅ is

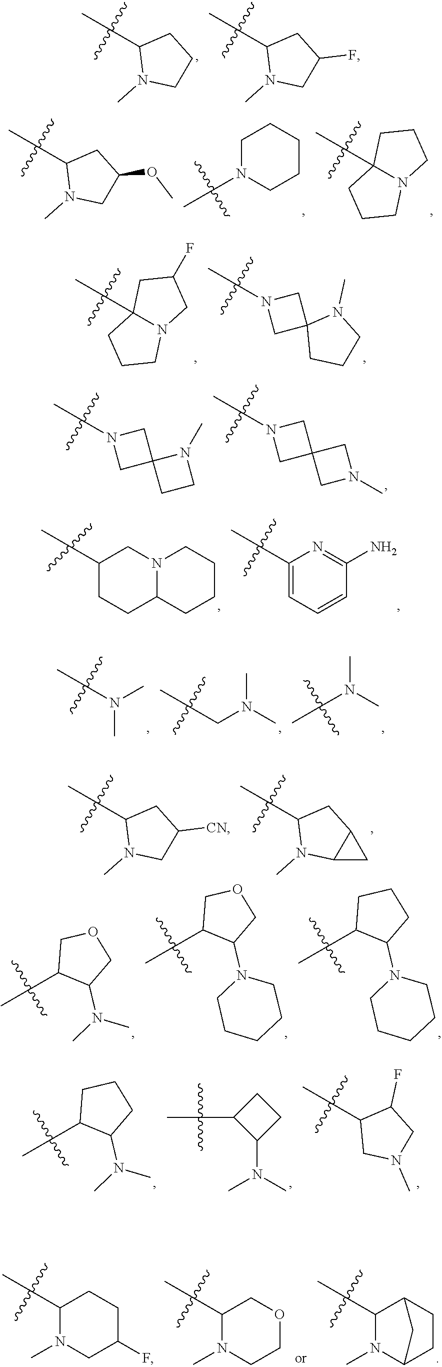

17. A compound selected from the group consisting of 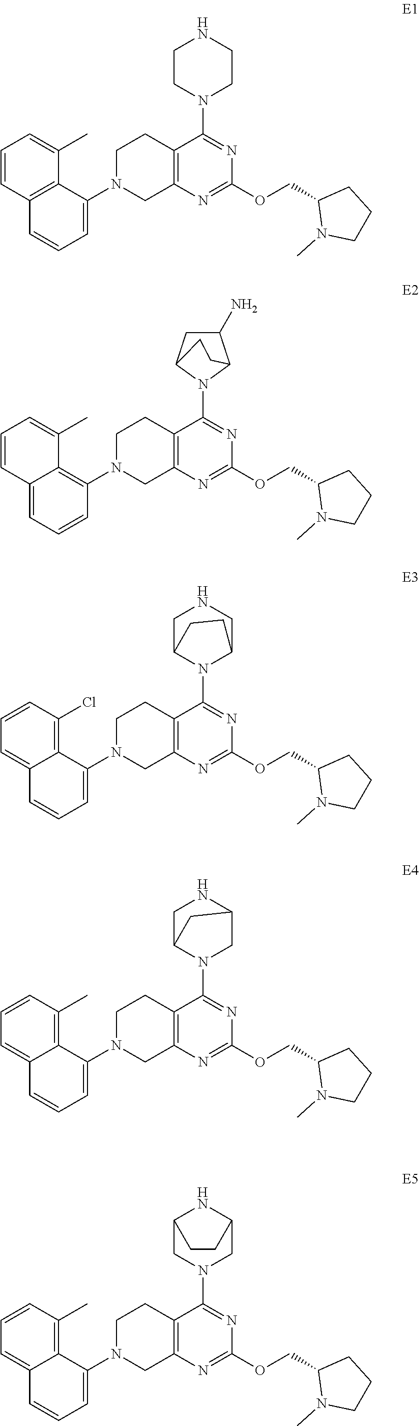 [SMILES: Cc1cccc2cccc(N3CCc4c(nc(OC[C@@H]5CCCN5C)nc4N4CCNCC4)C3)c12 ; Cc1cccc2cccc(N3CCc4c(nc(OC[C@@H]5CCCN5C)nc4N4C5CCC4C(N)C5)C3)c12 ; CN1CCC[C@H]1COc1nc2c(c(N3C4CCC3CNC4)n1)CCN(c1cccc3cccc(Cl)c13)C2 ; Cc1cccc2cccc(N3CCc4c(nc(OC[C@@H]5CCCN5C)nc4N4CC5CC4CN5)C3)c12 ; Cc1cccc2cccc(N3CCc4c(nc(OC[C@@H]5CCCN5C)nc4N4CC5CCC(C4)N5)C3)c12] 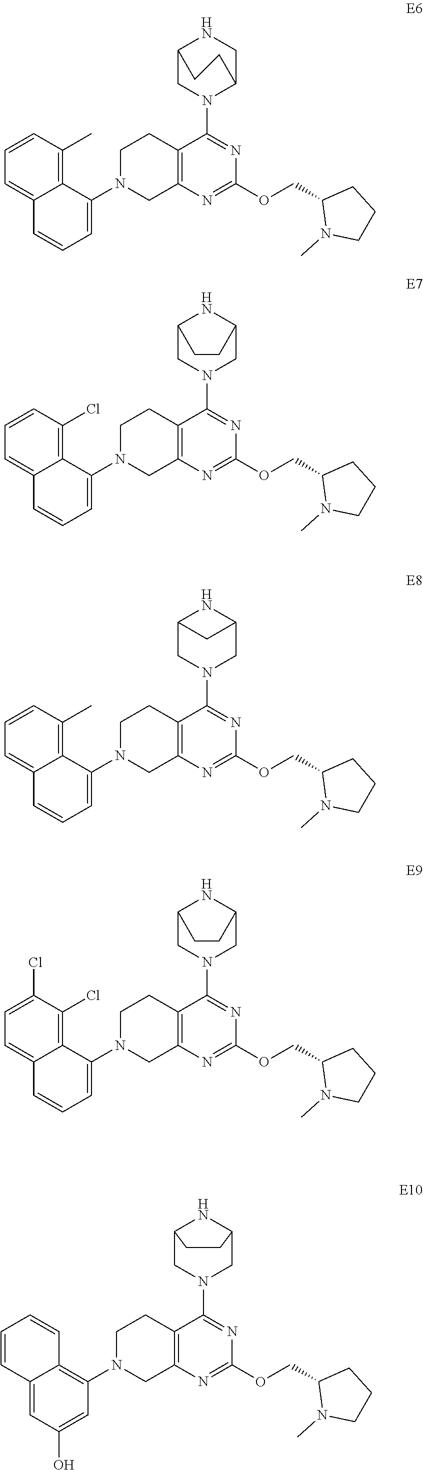 [SMILES: Cc1cccc2cccc(N3CCc4c(nc(OC[C@@H]5CCCN5C)nc4N4CC5CCC4CN5)C3)c12 ; CN1CCC[C@H]1COc1nc2c(c(N3CC4CCC(C3)N4)n1)CCN(c1cccc3cccc(Cl)c13)C2 ; Cc1cccc2cccc(N3CCc4c(nc(OC[C@@H]5CCCN5C)nc4N4CC5CC(C4)N5)C3)c12 ; CN1CCC[C@H]1COc1nc2c(c(N3CC4CCC(C3)N4)n1)CCN(c1cccc3ccc(Cl)c(Cl)c13)C2 ; CN1CCC[C@H]1COc1nc2c(c(N3CC4CCC(C3)N4)n1)CCN(c1cc(O)cc3ccccc13)C2] 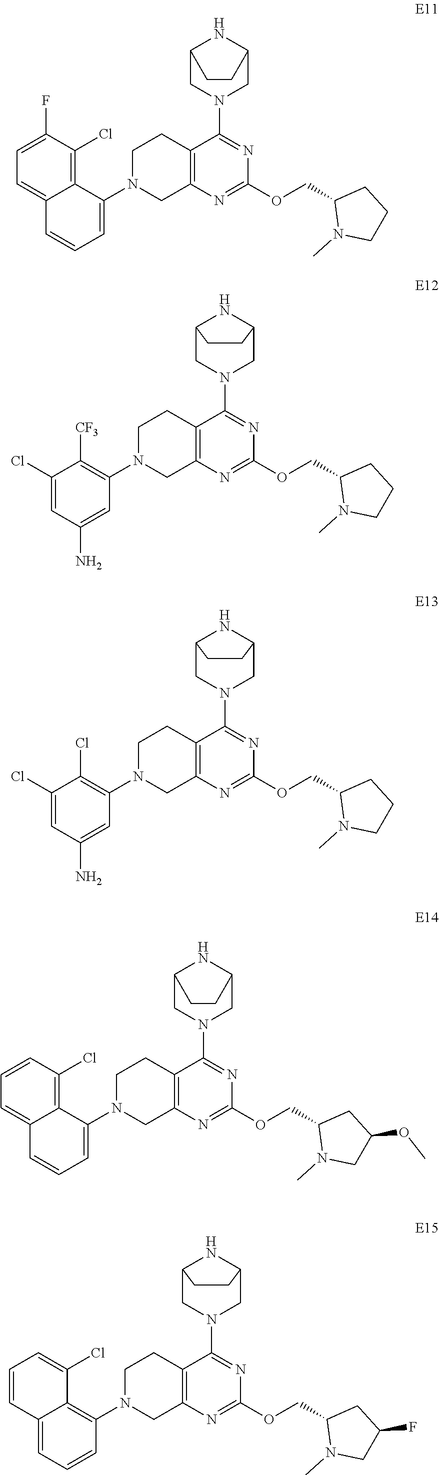 [SMILES: CN1CCC[C@H]1COc1nc2c(c(N3CC4CCC(C3)N4)n1)CCN(c1cccc3ccc(F)c(Cl)c13)C2 ; CN1CCC[C@H]1COc1nc2c(c(N3CC4CCC(C3)N4)n1)CCN(c1cc(N)cc(Cl)c1C(F)(F)F)C2 ; CN1CCC[C@H]1COc1nc2c(c(N3CC4CCC(C3)N4)n1)CCN(c1cc(N)cc(Cl)c1Cl)C2 ; CO[C@@H]1C[C@@H](COc2nc3c(c(N4CC5CCC(C4)N5)n2)CCN(c2cccc4cccc(Cl)c24)C3)N(C)C1 ; CN1C[C@H](F)C[C@H]1COc1nc2c(c(N3CC4CCC(C3)N4)n1)CCN(c1cccc3cccc(Cl)c13)C2] 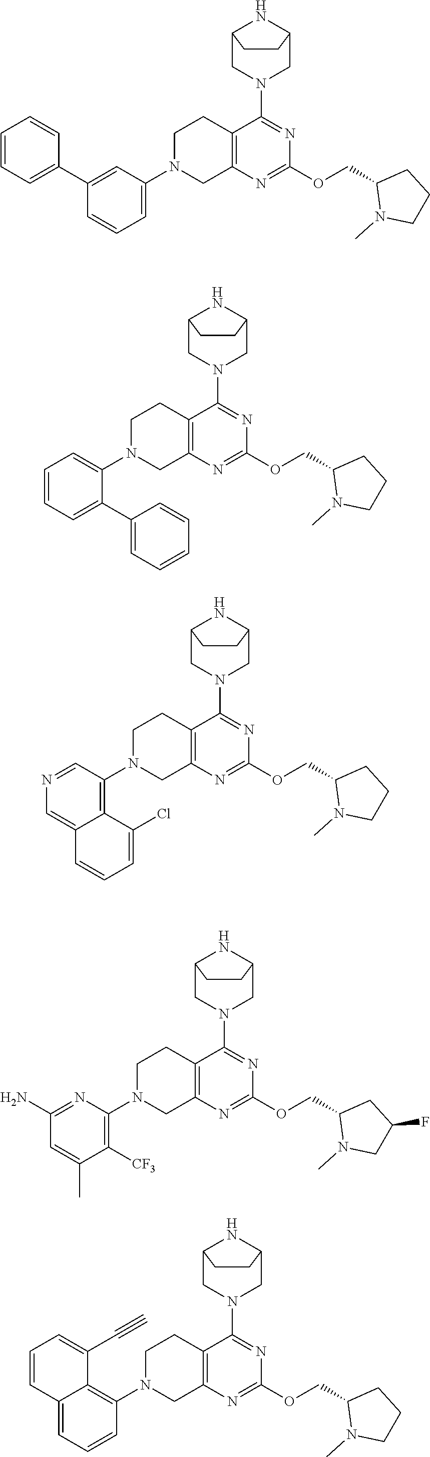 [SMILES: CN1CCC[C@H]1COc1nc2c(c(N3CC4CCC(C3)N4)n1)CCN(c1cccc(-c3ccccc3)c1)C2 ; CN1CCC[C@H]1COc1nc2c(c(N3CC4CCC(C3)N4)n1)CCN(c1ccccc1-c1ccccc1)C2 ; CN1CCC[C@H]1COc1nc2c(c(N3CC4CCC(C3)N4)n1)CCN(c1cncc3cccc(Cl)c13)C2 ; Cc1cc(N)nc(N2CCc3c(nc(OC[C@@H]4C[C@@H](F)CN4C)nc3N3CC4CCC(C3)N4)C2)c1C(F)(F)F ; C#Cc1cccc2cccc(N3CCc4c(nc(OC[C@@H]5CCCN5C)nc4N4CC5CCC(C4)N5)C3)c12] 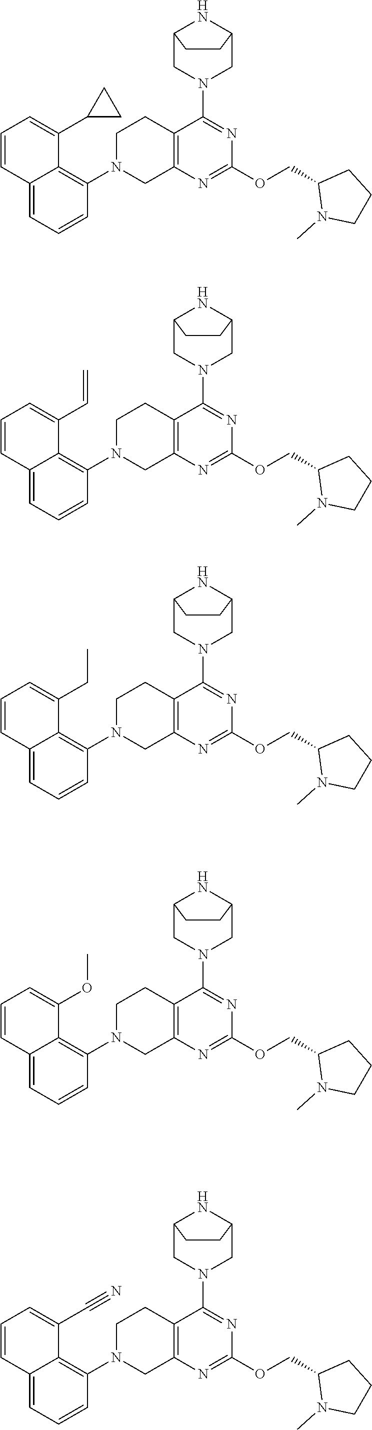 [SMILES: CN1CCC[C@H]1COc1nc2c(c(N3CC4CCC(C3)N4)n1)CCN(c1cccc3cccc(C4CC4)c13)C2 ; C=Cc1cccc2cccc(N3CCc4c(nc(OC[C@@H]5CCCN5C)nc4N4CC5CCC(C4)N5)C3)c12 ; CCc1cccc2cccc(N3CCc4c(nc(OC[C@@H]5CCCN5C)nc4N4CC5CCC(C4)N5)C3)c12 ; COc1cccc2cccc(N3CCc4c(nc(OC[C@@H]5CCCN5C)nc4N4CC5CCC(C4)N5)C3)c12 ; CN1CCC[C@H]1COc1nc2c(c(N3CC4CCC(C3)N4)n1)CCN(c1cccc3cccc(C#N)c13)C2] 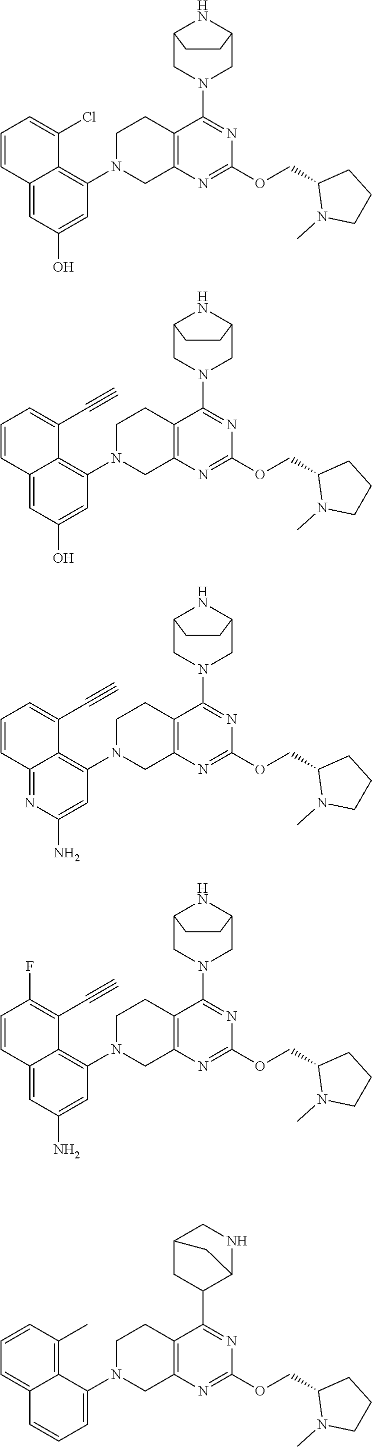 [SMILES: CN1CCC[C@H]1COc1nc2c(c(N3CC4CCC(C3)N4)n1)CCN(c1cc(O)cc3cccc(Cl)c13)C2 ; C#Cc1cccc2cc(O)cc(N3CCc4c(nc(OC[C@@H]5CCCN5C)nc4N4CC5CCC(C4)N5)C3)c12 ; C#Cc1cccc2nc(N)cc(N3CCc4c(nc(OC[C@@H]5CCCN5C)nc4N4CC5CCC(C4)N5)C3)c12 ; C#Cc1c(F)ccc2cc(N)cc(N3CCc4c(nc(OC[C@@H]5CCCN5C)nc4N4CC5CCC(C4)N5)C3)c12 ; Cc1cccc2cccc(N3CCc4c(nc(OC[C@@H]5CCCN5C)nc4C4CC5CNC4C5)C3)c12] 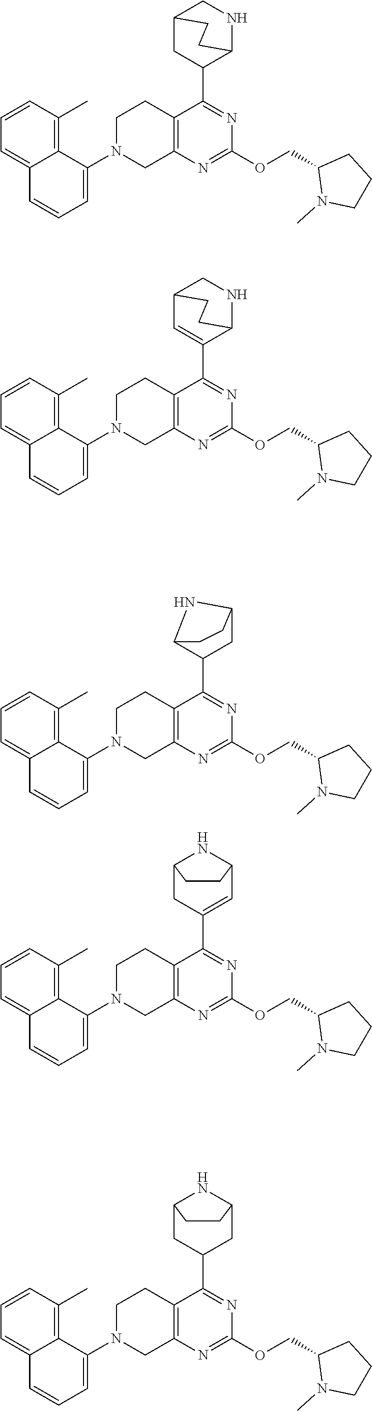 [SMILES: Cc1cccc2cccc(N3CCc4c(nc(OC[C@@H]5CCCN5C)nc4C4CC5CCC4NC5)C3)c12 ; Cc1cccc2cccc(N3CCc4c(nc(OC[C@@H]5CCCN5C)nc4C4=CC5CCC4NC5)C3)c12 ; Cc1cccc2cccc(N3CCc4c(nc(OC[C@@H]5CCCN5C)nc4C4CC5CCC4N5)C3)c12 ; Cc1cccc2cccc(N3CCc4c(nc(OC[C@@H]5CCCN5C)nc4C4=CC5CCC(C4)N5)C3)c12 ; Cc1cccc2cccc(N3CCc4c(nc(OC[C@@H]5CCCN5C)nc4C4CC5CCC(C4)N5)C3)c12] 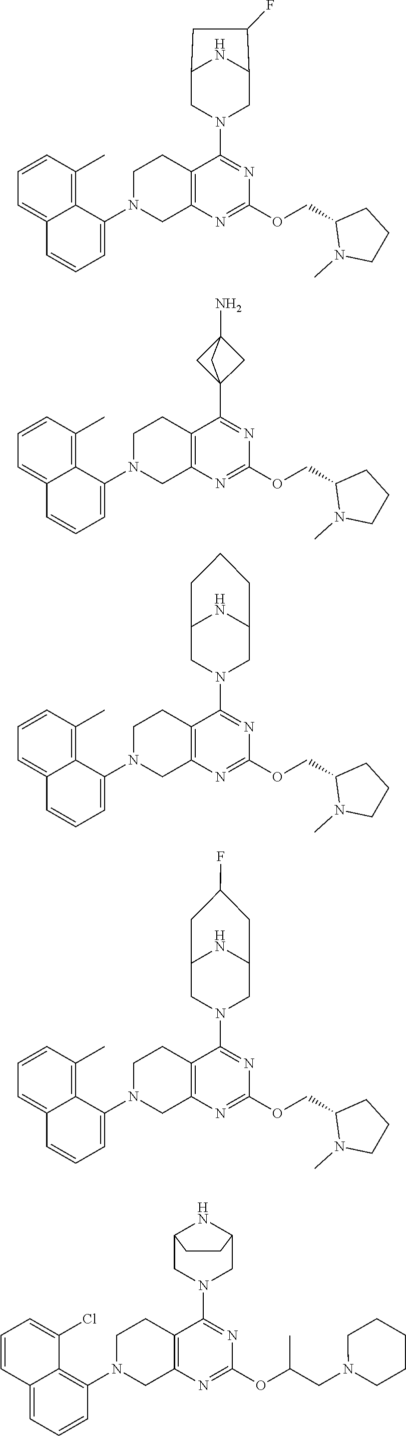 [SMILES: Cc1cccc2cccc(N3CCc4c(nc(OC[C@@H]5CCCN5C)nc4N4CC5CC(F)C(C4)N5)C3)c12 ; Cc1cccc2cccc(N3CCc4c(nc(OC[C@@H]5CCCN5C)nc4C45CC(N)(C4)C5)C3)c12 ; Cc1cccc2cccc(N3CCc4c(nc(OC[C@@H]5CCCN5C)nc4N4CC5CCCC(C4)N5)C3)c12 ; Cc1cccc2cccc(N3CCc4c(nc(OC[C@@H]5CCCN5C)nc4N4CC5CC(F)CC(C4)N5)C3)c12 ; CC(CN1CCCCC1)Oc1nc2c(c(N3CC4CCC(C3)N4)n1)CCN(c1cccc3cccc(Cl)c13)C2] 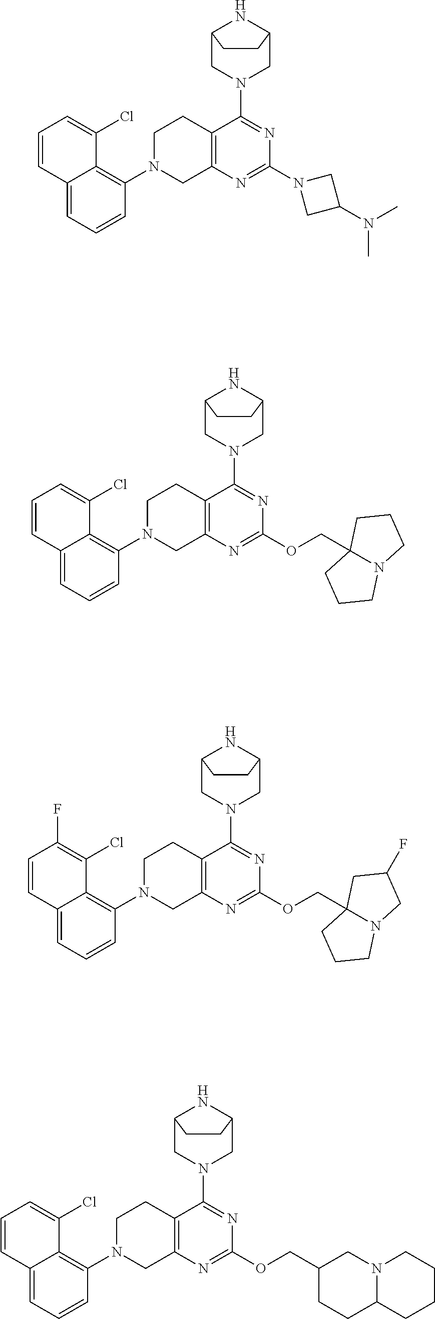 [SMILES: CN(C)C1CN(c2nc3c(c(N4CC5CCC(C4)N5)n2)CCN(c2cccc4cccc(Cl)c24)C3)C1 ; Clc1cccc2cccc(N3CCc4c(nc(OCC56CCCN5CCC6)nc4N4CC5CCC(C4)N5)C3)c12 ; Fc1ccc2cccc(N3CCc4c(nc(OCC56CCCN5CC(F)C6)nc4N4CC5CCC(C4)N5)C3)c2c1Cl ; Clc1cccc2cccc(N3CCc4c(nc(OCC5CCC6CCCCN6C5)nc4N4CC5CCC(C4)N5)C3)c12] 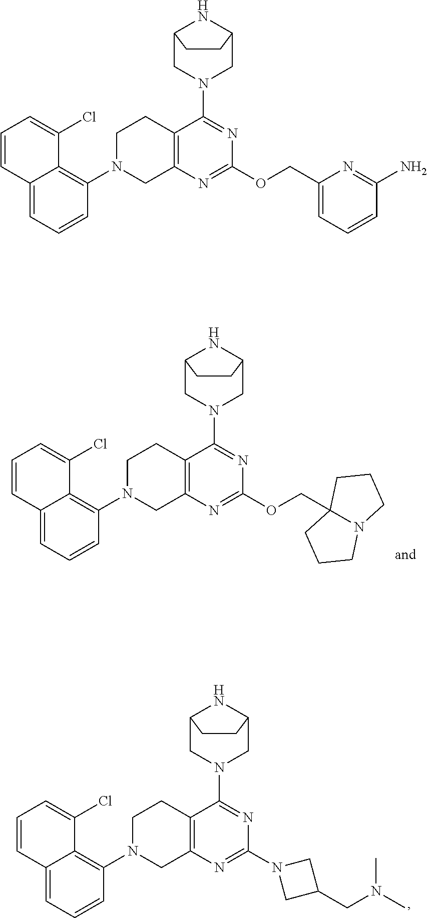 [SMILES: Nc1cccc(COc2nc3c(c(N4CC5CCC(C4)N5)n2)CCN(c2cccc4cccc(Cl)c24)C3)n1 ; Clc1cccc2cccc(N3CCc4c(nc(OCC56CCCN5CCC6)nc4N4CC5CCC(C4)N5)C3)c12 ; CN(C)CC1CN(c2nc3c(c(N4CC5CCC(C4)N5)n2)CCN(c2cccc4cccc(Cl)c24)C3)C1]
or a pharmaceutically acceptable salt thereof, or a stereoisomer thereof.

18. A pharmaceutical composition comprising the compound of claim 1 or a stereoisomer thereof, or a pharmaceutically acceptable salt thereof and a pharmaceutically acceptable excipient.

19. A method of treating cancer, comprising administering to a subject in need thereof a therapeutically effective amount of the compound of claim 1 or a stereoisomer thereof, or a pharmaceutically acceptable salt thereof.

20. (canceled)

21. (canceled)
